(19) United States
(12) Patent Application Publication
(10) Pub. No.: US 2026/0275348 A1
(43) Pub. Date: Sep. 17, 2026

(54) MODIFIED SINA MOLECULES, METHODS AND USES THEREOF

(71) Applicant: PHYZAT BIOPHARMACEUTICALS, LDA, Porto (PT)

(72) Inventor: Patrício SOARES DA SILVA, Porto (PT)

(73) Assignee: PHYZAT BIOPHARMACEUTICALS, LDA, Porto (PT)

(21) Appl. No.: 18/996,459

(22) PCT Filed: Aug. 1, 2023
(86) PCT No.: PCT/IB2023/057805

§ 371 Date: Jan. 17, 2025

(30) Foreign Application Priority Data
Aug. 1, 2022 (PT) 118143
Aug. 2, 2022 (PT) 118146

(51) Int. Cl. C12N 15/113; A61K 31/712; A61K 31/7125; A61P 27/06

(52) U.S. Cl. CPC C12N 15/1137; A61K 31/712; A61K 31/7125; A61P 27/06; C12N 2310/14; C12N 2310/315; C12N 2310/321; C12N 2310/322; C12N 2310/343; C12N 2310/346; C12N 2320/30; C12Y 114/17001

(57) ABSTRACT

The present disclosure relates to a double stranded short interfering nucleic acid (siNA) agent for use in medicine, in particular for use in the treatment of ophthalmic diseases comprising an antisense ribonucleotide sequence arranged to base-pair to a substantially complementary sense ribonucleotide sequence; wherein all of the nucleotides of the sense strand and all of the nucleotides of the antisense strand comprise a 2′-O-methyl modification or a 2′-fluoro modification; wherein both the sense strand and the antisense strand independently comprise at least one phosphorothioate internucleotide linkage; wherein the antisense ribonucleotide sequence is substantially complementary to a sense ribonucleotide sequence except in up to 6 nucleotides; wherein the sense strand comprises 15 to 21 nucleotides; wherein each nucleotide or each of the first 18 contiguous nucleotides in the 5′-3′ direction comprise a modification that is different from the modification of the adjacent nucleotide; and wherein the first modification in the antisense strand is a 2′-O-methyl modification.

### CROSS-REFERENCE TO RELATED APPLICATIONS

This application is a U.S. National Stage Application under 35 U.S.C. § 371 of International Patent Application No. PCT/IB2023/057805, filed Aug. 1, 2023, which claims priority to Portugal Patent Application No. 118143, filed Aug. 1, 2022 and Portugal Patent Application No. 118146 filed Aug. 2, 2022, the contents of which are hereby incorporated by reference in their respective entireties.

### SEQUENCE LISTING

The instant application contains a Sequence Listing which has been submitted electronically in XML format and is hereby incorporated by reference in its entirety. Said XML copy, created on Jun. 23, 2025, is named 10224_013015-US0_SL-v2.xml and is 3,293,624 bytes in size.

### TECHNICAL FIELD

The present disclosure relates to a composition for preventing and for use in medicine, in particular for use in the treatment of ophthalmic diseases, comprising short interfering nucleic acids (siNAs) to mediate gene silencing. The present disclosure is also directed to siNA duplexes, double stranded agents, to inhibit expression of dopamine-beta-hydroxylase (DBH, EC 1.14.17.1) genes and their use on methods of preventing and treating optical neuropathy associated with the elevation of intraocular pressure due to excessive noradrenergic activation.

### BACKGROUND

Glaucoma, the main cause of blindness in industrialized countries, is characterized by progressive optic neuropathy and irreversible visual field loss (Prokofyeva & Zrenner, 2012). Risk factors for developing glaucoma include elevated intraocular pressure (IOP), family history, ethnic background, and old age (Coleman & Miglior, 2008; Webers, Beckers, Nuijts & Schouten, 2008). Lowering IOP reduces the progression of nerve damage and therefore therapeutic management of glaucoma includes medications or surgeries that decrease IOP.

Primary open-angle glaucoma is one of the leading causes of irreversible blindness worldwide. It is characterized by a progressive loss of retinal ganglion cells (RGCs) and visual field damage (Quigley, 2011). Many studies have identified high IOP as the most important risk factor for retinal ganglion cell apoptosis in glaucoma (Davis, Crawley, Pahlitzsch, Javaid & Cordeiro, 2016). Clinical evidence has already shown that neuronal damage in glaucoma involves central stations of the visual pathway (Nucci et al., 2013). Recent magnetic resonance imaging studies have also suggested that primary open-angle glaucoma abnormalities are not limited to RGCs but extends to the entire visual pathway as well as some non-visual pathways (Frezzotti et al., 2014; Frezzotti, Giorgio, Toto, De Leucio & De Stefano, 2016).

Different subtypes of adrenoceptors appear to mediate the various ways noradrenaline could affect the progression of open-angle glaucoma. For example, noradrenaline has a constrictive effect on dog ocular arteries, possibly by acting through alpha1-adrenoceptors (Okamura, Fujioka & Ayajiki, 2002). The alpha2 adrenoceptor agonists, xylazine and clonidine, both produce mydriasis (excessive pupil dilation, which can reduce the drainage angle), possibly through postsynaptic agonism of alpha2 adrenoceptors (Hey, Gherezghiher & Koss, 1985; Hsu, Lee & Betts, 1981). Endogenous noradrenaline may act in a similar fashion through these receptors. Pharmacological blockade of beta adrenoceptors decreases aqueous humour production (Rittenhouse & Pollack, 2000), suggesting that if endogenous noradrenaline boosts humour production, it may do so through stimulation of beta adrenoceptors. Timolol significantly reduced IOP in saline control treated mice, but did not significantly affect IOP in the reserpine-treated animals. These findings demonstrate that catecholamines are required for the IOP-lowering effects of the beta adrenoceptor antagonist, timolol (Ishikawa, Yoshitomi, Zorumski & Izumi, 2015).

A second line of evidence for an etiological role of noradrenaline in open-angle glaucoma comes from studies of noradrenergic drugs in humans. The main point here is that two major classes of drugs that are used to treat open-angle glaucoma, beta-blockers and alpha2 adrenergic agonists, exert their effects directly on noradrenaline signalling. Examples of these drugs include the beta-blocker timolol and the alpha2-adrenoceptor agonist brimonidine, both mentioned above in preclinical studies (Burke et al., 1995; Greenfield, Liebmann & Ritch, 1997; Gupta, Agarwal, Galpalli, Srivastava, Agrawal & Saxena, 2007; Seki et al., 2005). Brimonidine has been widely used to treat open-angle glaucoma, and it is also used in combination with timolol (Fudemberg, Batiste & Katz, 2008).

Noradrenaline may be involved in other types of glaucoma as well. A first line of evidence involves rodent studies of glaucoma in the context of pharmacological noradrenaline manipulation. Drugs that affect noradrenaline signalling have been demonstrated to affect IOP in rodents as well as in rabbits. A number of studies have tested beta-blockers, such as timolol, in these animal models and found a reduction in IOP (Gupta, Agarwal, Galpalli, Srivastava, Agrawal & Saxena, 2007; Seki et al., 2005). Other studies have tested noradrenaline lowering alpha2-adrenoceptor agonists, such as brimonidine, and found a reduction in IOP (Burke et al., 1995; Greenfield, Liebmann & Ritch, 1997). In addition, brimonidine and clonidine exert neuroprotective effects on the retina (Ahmed, Hegazy, Chaudhary & Sharma, 2001; Wheeler & Woldemussie, 2001).

Given that excessive production of noradrenaline may be involved in open angle glaucoma and other types of glaucoma, inhibition of excessive noradrenaline production result in a valid alternative to therapies based on the modulation of noradrenaline-mediated effects through blockade of beta-adrenoceptors or activation of alpha2-adrenoceptors which lead to decreases in noradrenaline release from sympathetic nerve endings, as these do not alter the root cause of eye sympathetic noradrenergic overactivity.

The eye is a relatively isolated tissue compartment; this provides several advantages to the use of small interfering RNA (siRNA)-based therapies. Local delivery of compounds to the eye limits systemic exposure and reduces the amount of compound needed. It allows for local silencing of a gene while reducing the likelihood of wide spread silencing outside the eye. In addition, the immune system has limited access to the eye; therefore, immune responses to the compound are less likely to occur (Campochiaro, 2006). Finally, the eye has lower content in RNases than other tissues, allowing for an increased stability of RNA-based compounds (Martinez, Gonzalez, Roehl, Wright, Paneda & Jimenez, 2014).

RNA interference (“RNAi”) is a recently discovered mechanism of post-transcriptional gene silencing in which double-stranded RNA corresponding to a gene (or coding region) of interest is introduced into an organism, resulting in degradation of the corresponding mRNA. The phenomenon was originally discovered in Caenorhabditis elegans (Fire, Xu, Montgomery, Kostas, Driver & Mello, 1998). Unlike antisense technology, the RNAi phenomenon persists for multiple cell divisions before gene expression is regained. The process occurs in at least two steps: an endogenous ribonuclease cleaves the longer double stranded RNA (dsRNA) into shorter, 21-22- or 23-nucleotide-long RNAs, termed “small interfering RNAs” or siRNAs (Hannon, 2002). The siRNA segments then mediate the degradation of the target mRNA. RNAi has been used for gene function determination in a manner similar to but more efficient than antisense oligonucleotides. By making targeted knockouts at the RNA level by RNAi, rather than at the DNA level using conventional gene knockout technology, a vast number of genes can be assayed quickly and efficiently. RNAi is therefore an extremely powerful, simple method for assaying gene function.

RNAi has been shown to be effective in cultured mammalian cells. In most methods described to date, RNAi is carried out by introducing double-stranded RNA into cells by microinjection or by soaking cultured cells in a solution of double-stranded RNA, as well as transfecting the cells with a plasmid carrying a hairpin-structured siRNA expressing cassette under the control of suitable promoters, such as the U6, H1 or cytomegalovirus (“CMV”) promoter (Brummelkamp, Bernards & Agami, 2002; Elbashir, Harborth, Lendeckel, Yalcin, Weber &Tuschl, 2001; Harborth, Elbashir, Bechert, Tuschl & Weber, 2001; Lee et al., 2001; Miyagishi & Taira, 2002; Paddison, Caudy, Bernstein, Hannon & Conklin, 2002; Paul, Good, Winer & Engelke, 2002; Sui et al., 2002; Xia, Mao, Paulson & Davidson, 2002; Yu, DeRuiter & Turner, 2002). The gene-specific inhibition of gene expression by double-stranded ribonucleic acid is generally described in U.S. Pat. No. 6,506,559, which is incorporated herein by reference. Exemplary use of siRNA technology is further described in Published U.S. Patent Application No. 2003/01090635 and Published U.S. Patent Application No. 20040248174, which are incorporated herein by reference. Davis (Davis, 2009) describes the targeted delivery of siRNA to humans using nanoparticle technology.

Document WO2022107106 describes siNA molecules for use as a medicament, in particular for use in preventing and treating optical neuropathy associated with the elevation of intraocular pressure due to excessive noradrenergic activation.

These facts are disclosed in order to illustrate the technical problem addressed by the present disclosure.

### GENERAL DESCRIPTION

In an embodiment, the human dopamine-beta-hydroxylase (DBH, EC 1.14.17.1) protein is the protein as identified by the NCBI sequence reference GI:116534900 (GenBank Accession No. NP_000778.3; SEQ ID No. 1), as encoded by the nucleotide sequence identified by the NCBI sequence reference GI:1653961349 (GenBank Accession No. NM_000787.4—Homo sapiens DBH; SEQ ID No. 2; reverse complement, SEQ ID No. 3), homolog or functional fragment thereof (Table 1).

In another embodiment, the dopamine-beta-hydroxylase (DBH, EC 1.14.17.1) protein in other mammalian species is the protein in the monkey, dog, horse, bovine, cat, mouse and rat as identified by the NCBI sequence references GI:1783383999 (GenBank Accession No. NP_001265274.2; SEQ ID No. 4), GI:2161843713 (GenBank Accession No XP_005580554.2; SEQ ID No. 7), GI:2161843711 (GenBank Accession No XP_045228715.1; SEQ ID No. 10), GI:55742740 (GenBank Accession No NP_001005263.1; SEQ ID No. 13), GI:824556479 (GenBank Accession No NP_001075239.2; SEQ ID No. 16), GI:30794286 (GenBank Accession No NP_851338.1; SEQ ID No. 19), GI:2131045732 (GenBank Accession No XP_003996085.3; SEQ ID No. 22), GI:116534900 (GenBank Accession NP_620392.2; SEQ ID No. 25) and GI:1937883836 (GenBank Accession No NP_037290.3; SEQ ID No. 28),respectively, homologs or functional fragments thereof (Table 1).

Exemplary nucleotide and amino acid sequences of DBH can be found, for example, at GenBank Accession No. NM_001278345.2 (Macaca mulatta DBH, SEQ ID No. 6; reverse complement, SEQ ID No. 7); GenBank Accession No. XM_005580497.3 (Macaca fascicularis DBH, SEQ ID No. 8; reverse complement, SEQ ID No. 9); GenBank Accession No. XM_045372780.1 (Macaca fascicularis DBH, SEQ ID No. 11; reverse complement, SEQ ID No. 12); GenBank Accession No. NM_001005263.1 (Canis lupus familiaris DBH—SEQ ID No. 14; reverse complement, SEQ ID No. 15); GenBank Accession No. NM_001081770.3 (Equus caballus DBH, SEQ ID No. 17; reverse complement, SEQ ID No. 18); GenBank Accession No. NM_180995.2 (Bos taurus DBH, SEQ ID No. 20; reverse complement, SEQ ID No. 21); GenBank Accession No. XM_003996036.6 (Felis catus DBH, SEQ ID No. 23; reverse complement, SEQ ID No. 24); GenBank Accession No. NM_138942.3 (Mus musculus DBH—SEQ ID No. 26; reverse complement, SEQ ID No. 27); GenBank Accession No. NM_013158.3 (Rattus norvegicus DBH—SEQ ID No. 29; reverse complement, SEQ ID No. 30). The DBH gene multiple sequence alignment, concerning Mus musculus DBH gene (NM_138942.3), Rattus norvegicus DBH gene (NM_013158.3), Canis lupus familiaris DBH gene (NM_001005263.1), Felis catus DBH gene (XM_003996036.6), Bos taurus DBH gene (NM_180995.2), Equus caballus DBH gene (NM_001081770.3), human (Homo sapiens) DBH gene (NM_000787.4), Macaca mulatta DBH gene (NM_001278345.2) and Macaca fascicularis DBH gene (XM_005580497.3), was made using the CLUSTAL O(1.2.4) tool.

Additional examples of DBH mRNA sequences are readily available through publicly available databases, e.g., GenBank, UniProt, OMIM, and the dog, horse, bovine, monkey, mouse and rat genome project web sites. The DBH protein multiple sequence alignment, concerning Canis lupus familiaris DBH protein (NP_001005263.1), Mus musculus DBH protein (NP_037290.3), Rattus norvegicus DBH protein (NP_620392.2), human (Homo sapiens) DBH protein (NP_000778.3), Macaca mulatta DBH protein (NP_001265274.2), Macaca fascicularis DBH protein (XP_005580554.2), Felis catus DBH protein (XP_003996085.3), Equus caballus (NP_001075239.2) and Bos taurus DBH protein (NP_851338.1), was made using CLUSTAL O(1.2.4).

Further information on DBH can be found, for example, at www.ncbi.nlm.nih.gov/gene/?term=dbh.

The entire contents of each of the foregoing GenBank Accession numbers and the Gene database numbers are incorporated herein by reference as of the date of filing this application.

The term DBH, as used herein, also refers to variations of the DBH gene including variants provided in the Single Nucleotide Polymorphisms (SNP) database (dbSNP). Numerous sequence variations within the DBH gene have been identified and may be found at, for example, NCBI dbSNP and UniProt (see, e.g., www.ncbi.nlm.nih.gov/snp/?term=dbh), the entire contents of which is incorporated herein by reference as of the date of filing this application.

As used herein, “target sequence” refers to a contiguous portion of the nucleotide sequence of an mRNA molecule formed during the transcription of a DBH gene, including mRNA that is a product of RNA processing of a primary transcription product. The target portion of the sequence will be at least long enough to serve as a substrate for RNA-directed cleavage at or near that portion of the nucleotide sequence of an mRNA molecule formed during the transcription of a DBH gene. In one embodiment, the target sequence is within the protein coding region of DBH.

The target sequence may be from about 19-36 nucleotides in length, e.g., preferably about 19-30 nucleotides in length. For example, the target sequence can be about 19-30 nucleotides, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length. In certain embodiments, the target sequence is 19-23 nucleotides in length, optionally 21-23 nucleotides in length. Ranges and lengths intermediate to the above recited ranges and lengths are also contemplated to be part of the disclosure.

As used herein, the term “strand comprising a sequence” refers to an oligonucleotide comprising a chain of nucleotides that is described by the sequence referred to using the standard nucleotide nomenclature.

“G,” “C,” “A,” “T,” and “U” each generally stand for a nucleotide that contains guanine, cytosine, adenine, thymidine, and uracil as a base, respectively. However, it will be understood that the term “ribonucleotide” or “nucleotide” can also refer to a modified nucleotide, as further detailed below, or a surrogate replacement moiety (see, e.g., Table 2). The skilled person is well aware that guanine, cytosine, adenine, and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in the nucleotide sequences of dsRNA featured in the invention by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods featured in the present disclosure.

The present disclosure relates to a double stranded short interfering nucleic acid (siNA) agent for use in medicine comprising an antisense ribonucleotide sequence arranged to base-pair to a substantially complementary sense ribonucleotide sequence; wherein all of the nucleotides of the sense strand and all of the nucleotides of the antisense strand comprise a 2′-O-methyl modification or a 2′-fluoro modification; wherein both the sense strand and the antisense strand independently comprise at least one phosphorothioate internucleotide linkage; wherein the antisense ribonucleotide sequence is substantially complementary to a sense ribonucleotide sequence except in up to 6 nucleotides; wherein the sense strand comprises 15 to 21 nucleotides; wherein each nucleotide or each of the first 18 contiguous nucleotides in the 5′-3′ direction comprise a modification that is different from the modification of the adjacent nucleotide; and wherein the first modification in the antisense strand is a 2′-O-methyl modification. Preferably for use in the treatment of ophthalmic diseases.

In an embodiment, the modification of each nucleotide is different from the modification of the adjacent and of the complementary nucleotide.

In an embodiment, the 3 nucleotides adjacent to 3′ comprise the following modifications in the 5′-3′ direction:
2′-O-methyl modification; 2′-O-methyl modification and 2′-fluoro modification or;
2′-fluoro modification; 2′-O-methyl modification and 2′-O-methyl modification or;
2′-O-methyl modification; 2′-O-methyl modification and 2′-O-methyl modification; 2′-O-methyl modification.

In an embodiment, the siNA agent is for use in the treatment or prevention of ophthalmic diseases, preferably ophthalmic diseases susceptible of being improved or prevented by inhibition of noradrenaline production. In a further embodiment, the siNA agent is for use in the treatment or prevention of ophthalmic diseases associated with the elevation of IPO due to excessive noradrenergic activation.

For the scope and interpretation of the present disclosure the term “ophthalmic diseases” relates to diseases that affect the eyes of human or non-human animals. Thus, in an embodiment, the siNA agent is for use in the treatment or prevention of ophthalmic diseases, preferably ophthalmic diseases susceptible of being improved or prevented by inhibition of noradrenaline production in humans. In another embodiment, the siNA agent is for use in the treatment or prevention of ophthalmic diseases, preferably ophthalmic diseases susceptible of being improved or prevented by inhibition of noradrenaline production in non-human animals, preferably non-human mammals, more preferably monkey, bovine, cat, mice, mouse, dog or horse; more preferably dog or horse.

In an embodiment, the antisense ribonucleotide sequence is at least 90% identical to the sequences of the following list: SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95, SEQ ID No. 96, SEQ ID No. 97, SEQ ID No. 98, SEQ ID No. 99, SEQ ID No. 100, SEQ ID No. 101, SEQ ID No. 102, SEQ ID No. 103, SEQ ID No. 104, SEQ ID No. 105, SEQ ID No. 106, SEQ ID No. 107, SEQ ID No. 108, SEQ ID No. 109, SEQ ID No. 110, SEQ ID No. 176, SEQ ID No. 177, SEQ ID No. 178, SEQ ID No. 179, SEQ ID No. 180, SEQ ID No. 181, SEQ ID No. 182, SEQ ID No. 183, SEQ ID No. 184, SEQ ID No. 185, SEQ ID No. 186, SEQ ID No. 187, SEQ ID No. 188, SEQ ID No. 189, SEQ ID No. 190, SEQ ID No. 191, SEQ ID No. 192, SEQ ID No. 193, SEQ ID No. 194, SEQ ID No. 195, SEQ ID No. 196, SEQ ID No. 197, SEQ ID No. 198, SEQ ID No. 199, SEQ ID No. 200, SEQ ID No. 201, SEQ ID No. 202, SEQ ID No. 203, SEQ ID No. 204, SEQ ID No. 205, SEQ ID No. 206, SEQ ID No. 207, SEQ ID No. 208, SEQ ID No. 209, SEQ ID No. 210, SEQ ID No. 211, SEQ ID No. 212, SEQ ID No. 213, SEQ ID No. 214, SEQ ID No. 215, SEQ ID No. 216, SEQ ID No. 217, SEQ ID No. 218, SEQ ID No. 219, SEQ ID No. 220, SEQ ID No. 221, SEQ ID No. 222; the sense ribonucleotide sequence that is complementary to the antisense ribonucleotide sequence is at least 90% identical to the sequences of the following list: SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 167, SEQ ID No. 168, SEQ ID No. 169, SEQ ID No. 170, SEQ ID No. 171, SEQ ID No. 172, SEQ ID No. 173, SEQ ID No. 174, SEQ ID No. 175.

In another embodiment, the antisense ribonucleotide sequence is at least 90% identical to the sequences of the following list: SEQ ID No. 143, SEQ ID No. 144, SEQ ID No. 145, SEQ ID No. 146, SEQ ID No. 147, SEQ ID No. 148, SEQ ID No. 149, SEQ ID No. 150, SEQ ID No. 151, SEQ ID No. 152, SEQ ID No. 153, SEQ ID No. 154, SEQ ID No. 155, SEQ ID No. 156, SEQ ID No. 157, SEQ ID No. 158, SEQ ID No. 159, SEQ ID No. 160, SEQ ID No. 161, SEQ ID No. 162, SEQ ID No. 163, SEQ ID No. 164, SEQ ID No. 165, SEQ ID No. 166, SEQ ID No. 282, SEQ ID No. 283, SEQ ID No. 284, SEQ ID No. 285, SEQ ID No. 286, SEQ ID No. 287, SEQ ID No. 288, SEQ ID No. 289, SEQ ID No. 290, SEQ ID No. 291, SEQ ID No. 292, SEQ ID No. 293, SEQ ID No. 294, SEQ ID No. 295, SEQ ID No. 296, SEQ ID No. 297, SEQ ID No. 298, SEQ ID No. 299, SEQ ID No. 300, SEQ ID No. 301, SEQ ID No. 302, SEQ ID No. 303, SEQ ID No. 304, SEQ ID No. 305, SEQ ID No. 306, SEQ ID No. 307, SEQ ID No. 308, SEQ ID No. 309, SEQ ID No. 310, SEQ ID No. 311, SEQ ID No. 312, SEQ ID No. 313, SEQ ID No. 314, SEQ ID No. 315, SEQ ID No. 316, SEQ ID No. 317, SEQ ID No. 318, SEQ ID No. 319, SEQ ID No. 320, SEQ ID No. 321, SEQ ID No. 322, SEQ ID No. 323, SEQ ID No. 324, SEQ ID No. 325, SEQ ID No. 326; the sense ribonucleotide sequence is at least 90% identical to the sequences of the following list: SEQ ID No. 119, SEQ ID No. 120, SEQ ID No. 121, SEQ ID No. 122, SEQ ID No. 123, SEQ ID No. 124, SEQ ID No. 125, SEQ ID No. 126, SEQ ID No. 127, SEQ ID No. 128, SEQ ID No. 129, SEQ ID No. 130, SEQ ID No. 131, SEQ ID No. 132, SEQ ID No. 133, SEQ ID No. 134, SEQ ID No. 135, SEQ ID No. 136, SEQ ID No. 137, SEQ ID No. 138, SEQ ID No. 139, SEQ ID No. 140, SEQ ID No. 141 SEQ ID No. 142, SEQ ID No. 237, SEQ ID No. 238, SEQ ID No. 239, SEQ ID No. 240, SEQ ID No. 241, SEQ ID No. 242, SEQ ID No. 243, SEQ ID No. 244, SEQ ID No. 245, SEQ ID No. 246, SEQ ID No. 247, SEQ ID No. 248, SEQ ID No. 249, SEQ ID No. 250, SEQ ID No. 251, SEQ ID No. 252, SEQ ID No. 253, SEQ ID No. 254, SEQ ID No. 255, SEQ ID No. 256, SEQ ID No. 257, SEQ ID No. 258, SEQ ID No. 259, SEQ ID No. 260, SEQ ID No. 261, SEQ ID No. 262, SEQ ID No. 263, SEQ ID No. 264, SEQ ID No. 265, SEQ ID No. 266, SEQ ID No. 267, SEQ ID No. 268, SEQ ID No. 269, SEQ ID No. 270, SEQ ID No. 271, SEQ ID No. 272, SEQ ID No. 273, SEQ ID No. 274, SEQ ID No. 275, SEQ ID No. 276, SEQ ID No. 277, SEQ ID No. 278, SEQ ID No. 279, SEQ ID No. 280, SEQ ID No. 281.

In an embodiment, the antisense ribonucleotide sequence is at least 90% identical to the sequences of the following list: SEQ ID No. 89, SEQ ID No. 90, SEQ ID No. 91, SEQ ID No. 92, SEQ ID No. 93, SEQ ID No. 97, SEQ ID No. 98, SEQ ID No. 99, SEQ ID No. 101, SEQ ID No. 105, SEQ ID No. 106, SEQ ID No. 107, SEQ ID No. 109, SEQ ID No. 145, SEQ ID No. 146, SEQ ID No. 147, SEQ ID No. 149, SEQ ID No. 153, SEQ ID No. 154, SEQ ID No. 155, SEQ ID No. 157, SEQ ID No. 161, SEQ ID No. 162, SEQ ID No. 163, SEQ ID No. 165; the sense ribonucleotide sequence is at least 90% identical to the sequences of the following list: SEQ ID No. 81, SEQ ID No. 82, SEQ ID No. 83, SEQ ID No. 85, SEQ ID No. 121, SEQ ID No. 122, SEQ ID No. 123, SEQ ID No. 125, SEQ ID No. 129, SEQ ID No. 130, SEQ ID No. 131, SEQ ID No. 133, SEQ ID No. 137, SEQ ID No. 138, SEQ ID No. 139, SEQ ID No. 141.

In an embodiment, at least one strand is the sense strand.

In an embodiment, the siNA agent is for use in the prevention or reversing of progressive optical neuropathy, wherein the optical neuropathy is selected from the following list: diabetic retinopathy, infections, inflammation, uveitis and glaucoma, such as open-angle glaucoma, close-angle glaucoma, normal pressure glaucoma, congenital glaucoma, secondary glaucoma, pigmentary glaucoma, pseudoexfoliative glaucoma, traumatic glaucoma, neovascular glaucoma, endothelial iridocorneal syndrome and uveitic glaucoma.

In an embodiment, the sense strand is between 15 and 21 nucleotides in length.

In an embodiment the siNA is siRNA. In a further embodiment, the siNA is asymmetric.

Another aspect of the present disclosure relates to an agent for use in the treatment of ophthalmic diseases comprising a ribonucleotide sequence, wherein all the nucleotides of the ribonucleotide sequence comprise a modification selected from the group consisting of a 2′-O-methyl modification and a 2′-fluoro modification, wherein the modification of each nucleotide is different from the modification of the adjacent nucleotide, and wherein the first modification in the strand is a 2′-O-methyl modification.

Another aspect of the present disclosure relates to a pharmaceutical composition comprising at least one siNA agent as described in the present disclosure, and a pharmaceutically acceptable carrier.

In an embodiment, said composition is administered by topical eye application, subconjunctival injection, intravitreal injection, retrobulbar injection, intracameral injection, subtenon injection or deposition, intravenous injection, intravenous infusion.

The present disclosure relates to a double stranded short interfering nucleic acid (siNA) agent for use in the treatment of ophthalmic diseases comprising an antisense ribonucleotide sequence arranged to base-pair to a substantially complementary sense ribonucleotide sequence; wherein all of the nucleotides of the sense strand and all of the nucleotides of the antisense strand comprise a 2′-O-methyl modification or a 2′-fluoro modification; wherein both the sense strand and the antisense strand independently comprise at least one phosphorothioate internucleotide linkage; wherein the antisense ribonucleotide sequence is substantially complementary to a sense ribonucleotide sequence except in up to 6 nucleotides; wherein the sense strand comprises 15 to 21 nucleotides; wherein each nucleotide or each of the first 18 contiguous nucleotides in the 5′-3′ direction comprise a modification that is different from the modification of the adjacent nucleotide; and wherein the first modification in the antisense strand is a 2′-O-methyl modification.

In an embodiment, the antisense ribonucleotide sequence is at least 90% identical to the sequences of the following list: SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95, SEQ ID No. 96, SEQ ID No. 97, SEQ ID No. 98, SEQ ID No. 99, SEQ ID No. 100, SEQ ID No. 101, SEQ ID No. 102, SEQ ID No. 103, SEQ ID No. 104, SEQ ID No. 105, SEQ ID No. 106, SEQ ID No. 107, SEQ ID No. 108, SEQ ID No. 109, SEQ ID No. 110; SEQ ID No. 143, SEQ ID No. 144, SEQ ID No. 145, SEQ ID No. 146, SEQ ID No. 147, SEQ ID No. 148, SEQ ID No. 149, SEQ ID No. 150, SEQ ID No. 151, SEQ ID No. 152, SEQ ID No. 153, SEQ ID No. 154, SEQ ID No. 155, SEQ ID No. 156, SEQ ID No. 157, SEQ ID No. 158, SEQ ID No. 159, SEQ ID No. 160, SEQ ID No. 161, SEQ ID No. 162, SEQ ID No. 163, SEQ ID No. 164, SEQ ID No. 165, SEQ ID No. 166, SEQ ID No. 176, SEQ ID No. 177, SEQ ID No. 178, SEQ ID No. 179, SEQ ID No. 180, SEQ ID No. 181, SEQ ID No. 182, SEQ ID No. 183, SEQ ID No. 184, SEQ ID No. 185, SEQ ID No. 186, SEQ ID No. 187, SEQ ID No. 188, SEQ ID No. 189, SEQ ID No. 190, SEQ ID No. 191, SEQ ID No. 192, SEQ ID No. 193, SEQ ID No. 194, SEQ ID No. 195, SEQ ID No. 196, SEQ ID No. 197, SEQ ID No. 198, SEQ ID No. 199, SEQ ID No. 200, SEQ ID No. 201, SEQ ID No. 202, SEQ ID No. 203, SEQ ID No. 204, SEQ ID No. 205, SEQ ID No. 206, SEQ ID No. 207, SEQ ID No. 208, SEQ ID No. 209, SEQ ID No. 210, SEQ ID No. 211, SEQ ID No. 212, SEQ ID No. 213, SEQ ID No. 214, SEQ ID No. 215, SEQ ID No. 216, SEQ ID No. 217, SEQ ID No. 218, SEQ ID No. 219, SEQ ID No. 220, SEQ ID No. 221, SEQ ID No. 222; SEQ ID No. 282, SEQ ID No. 283, SEQ ID No. 284, SEQ ID No. 285, SEQ ID No. 286, SEQ ID No. 287, SEQ ID No. 288, SEQ ID No. 289, SEQ ID No. 290, SEQ ID No. 291, SEQ ID No. 292, SEQ ID No. 293, SEQ ID No. 294, SEQ ID No. 295, SEQ ID No. 296, SEQ ID No. 297, SEQ ID No. 298, SEQ ID No. 299, SEQ ID No. 300, SEQ ID No. 301, SEQ ID No. 302, SEQ ID No. 303, SEQ ID No. 304, SEQ ID No. 305, SEQ ID No. 306, SEQ ID No. 307, SEQ ID No. 308, SEQ ID No. 309, SEQ ID No. 310, SEQ ID No. 311, SEQ ID No. 312, SEQ ID No. 313, SEQ ID No. 314, SEQ ID No. 315, SEQ ID No. 316, SEQ ID No. 317, SEQ ID No. 318, SEQ ID No. 319, SEQ ID No. 320, SEQ ID No. 321, SEQ ID No. 322, SEQ ID No. 323, SEQ ID No. 324, SEQ ID No. 325, SEQ ID No. 326, preferably 91%, 92%, 93%, 94%, 95%, 96%, 97%, 98%, 99% or 100% identical; and the sense ribonucleotide sequence that is complementary to the antisense ribonucleotide sequence is at least 90% identical to the sequences of the following list: SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 119, SEQ ID No. 120, SEQ ID No. 121, SEQ ID No. 122, SEQ ID No. 123, SEQ ID No. 124, SEQ ID No. 125, SEQ ID No. 126, SEQ ID No. 127, SEQ ID No. 128, SEQ ID No. 129, SEQ ID No. 130, SEQ ID No. 131, SEQ ID No. 132, SEQ ID No. 133, SEQ ID No. 134, SEQ ID No. 135, SEQ ID No. 136, SEQ ID No. 137, SEQ ID No. 138, SEQ ID No. 139, SEQ ID No. 140, SEQ ID No. 141, SEQ ID No. 142, SEQ ID No. 167, SEQ ID No. 168, SEQ ID No. 169, SEQ ID No. 170, SEQ ID No. 171, SEQ ID No. 172, SEQ ID No. 173, SEQ ID No. 174, SEQ ID No. 175, SEQ ID No. 237, SEQ ID No. 238, SEQ ID No. 239, SEQ ID No. 240, SEQ ID No. 241, SEQ ID No. 242, SEQ ID No. 243, SEQ ID No. 244, SEQ ID No. 245, SEQ ID No. 246, SEQ ID No. 247, SEQ ID No. 248, SEQ ID No. 249, SEQ ID No. 250, SEQ ID No. 251, SEQ ID No. 252, SEQ ID No. 253, SEQ ID No. 254, SEQ ID No. 255, SEQ ID No. 256, SEQ ID No. 257, SEQ ID No. 258, SEQ ID No. 259, SEQ ID No. 260, SEQ ID No. 261, SEQ ID No. 262, SEQ ID No. 263, SEQ ID No. 264, SEQ ID No. 265, SEQ ID No. 266, SEQ ID No. 267, SEQ ID No. 268, SEQ ID No. 269, SEQ ID No. 270, SEQ ID No. 271, SEQ ID No. 272, SEQ ID No. 273, SEQ ID No. 274, SEQ ID No. 275, SEQ ID No. 276, SEQ ID No. 277, SEQ ID No. 278, SEQ ID No. 279, SEQ ID No. 280, SEQ ID No. 281, preferably 91%, 92%, 93%, 94%, 95%, 96%, 97%, 98%, 99% or 100% identical.

In an embodiment, the antisense ribonucleotide sequence is at least 90% identical to the sequences of the following list: SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95, SEQ ID No. 96, SEQ ID No. 97, SEQ ID No. 98, SEQ ID No. 99, SEQ ID No. 100, SEQ ID No. 101, SEQ ID No. 102, SEQ ID No. 103, SEQ ID No. 104, SEQ ID No. 105, SEQ ID No. 106, SEQ ID No. 107, SEQ ID No. 108, SEQ ID No. 109, SEQ ID No. 110, SEQ ID No. 143, SEQ ID No. 144, SEQ ID No. 145, SEQ ID No. 146, SEQ ID No. 147, SEQ ID No. 148, SEQ ID No. 149, SEQ ID No. 150, SEQ ID No. 151, SEQ ID No. 152, SEQ ID No. 153, SEQ ID No. 154, SEQ ID No. 155, SEQ ID No. 156, SEQ ID No. 157, SEQ ID No. 158, SEQ ID No. 159, SEQ ID No. 160, SEQ ID No. 161, SEQ ID No. 162, SEQ ID No. 163, SEQ ID No. 164, SEQ ID No. 165, SEQ ID No. 166, preferably 91%, 92%, 93%, 94%, 95%, 96%, 97%, 98%, 99% or 100% identical; and the sense ribonucleotide sequence is at least 90% identical to the sequences of the following list: SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 119, SEQ ID No. 120, SEQ ID No. 121, SEQ ID No. 122, SEQ ID No. 123, SEQ ID No. 124, SEQ ID No. 125, SEQ ID No. 126, SEQ ID No. 127, SEQ ID No. 128, SEQ ID No. 129, SEQ ID No. 130, SEQ ID No. 131, SEQ ID No. 132, SEQ ID No. 133, SEQ ID No. 134, SEQ ID No. 135, SEQ ID No. 136, SEQ ID No. 137, SEQ ID No. 138, SEQ ID No. 139, SEQ ID No. 140, SEQ ID No. 141, SEQ ID No. 142, preferably 91%, 92%, 93%, 94%, 95%, 96%, 97%, 98%, 99% or 100% identical.

In yet another embodiment, the antisense ribonucleotide sequence is at least 90% identical to the sequences of the following list: SEQ ID No. 89, SEQ ID No. 90, SEQ ID No. 91, SEQ ID No. 93, SEQ ID No. 97, SEQ ID No. 98, SEQ ID No. 99, SEQ ID No. 101, SEQ ID No. 105, SEQ ID No. 106, SEQ ID No. 107, SEQ ID No. 109, SEQ ID No. 145, SEQ ID No. 146, SEQ ID No. 147, SEQ ID No. 149, SEQ ID No. 153, SEQ ID No. 154, SEQ ID No. 155, SEQ ID No. 157, SEQ ID No. 161, SEQ ID No. 162, SEQ ID No. 163, SEQ ID No. 165, preferably 91%, 92%, 93%, 94%, 95%, 96%, 97%, 98%, 99% or 100% identical; and the sense ribonucleotide sequence is at least 90% identical to the sequences of the following list: SEQ ID No. 81, SEQ ID No. 82, SEQ ID No. 83, SEQ ID No. 85, SEQ ID No. 121, SEQ ID No. 122, SEQ ID No. 123, SEQ ID No. 125, SEQ ID No. 129, SEQ ID No. 130, SEQ ID No. 131, SEQ ID No. 133, SEQ ID No. 137, SEQ ID No. 138, SEQ ID No. 139, SEQ ID No. 141, preferably 91%, 92%, 93%, 94%, 95%, 96%, 97%, 98%, 99% or 100% identical.

In an embodiment, the modification of each nucleotide is different from the modification of the adjacent and of the complementary nucleotide; and the first modification in the antisense strand is a 2′-O-methyl modification.

In an embodiment, the 3 nucleotides adjacent to 3′ comprise the following modifications in the 5′-3′ direction:
2′-O-methyl modification; 2′-O-methyl modification and 2′-fluoro modification or;
2′-fluoro modification; 2′-O-methyl modification and 2′-O-methyl modification or;
2′-O-methyl modification; 2′-O-methyl modification and 2′-O-methyl modification; 2′-O-methyl modification.

In an embodiment, the siNA agent is for use in the treatment or prevention of ophthalmic diseases susceptible of being improved or prevented by inhibition of noradrenaline production.

In an embodiment, the siNA agent is for use in the treatment or prevention of progressive optical neuropathy associated with the elevation of intraocular pressure, preferably diabetic retinopathy, infections, inflammation, uveitis or glaucoma.

In an embodiment, the siNA is selected from double stranded RNA (dsRNA), small interfering RNAs (siRNA) or short hairpin RNA (shRNA). In a preferred embodiment, the siNA is siRNA.

In an embodiment, the siNA is asymmetric.

In an embodiment, the siNA agent is for use in the treatment of ophthalmic diseases in humans, wherein the antisense ribonucleotide sequence is at least 90% identical to the sequences of the following list: SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95, SEQ ID No. 96, SEQ ID No. 97, SEQ ID No. 98, SEQ ID No. 99, SEQ ID No. 100, SEQ ID No. 101, SEQ ID No. 102, SEQ ID No. 103, SEQ ID No. 104, SEQ ID No. 105, SEQ ID No. 106, SEQ ID No. 107, SEQ ID No. 108, SEQ ID No. 109, SEQ ID No. 110; SEQ ID No. 143, SEQ ID No. 144, SEQ ID No. 145, SEQ ID No. 146, SEQ ID No. 147, SEQ ID No. 148, SEQ ID No. 149, SEQ ID No. 150, SEQ ID No. 151, SEQ ID No. 152, SEQ ID No. 153, SEQ ID No. 154, SEQ ID No. 155, SEQ ID No. 156, SEQ ID No. 157, SEQ ID No. 158, SEQ ID No. 159, SEQ ID No. 160, SEQ ID No. 161, SEQ ID No. 162, SEQ ID No. 163, SEQ ID No. 164, SEQ ID No. 165, SEQ ID No. 166, preferably 91%, 92%, 93%, 94%, 95%, 96%, 97%, 98%, 99% or 100% identical; and the sense ribonucleotide sequence that is complementary to the antisense ribonucleotide sequence is at least 90% identical to the sequences of the following list: SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 119, SEQ ID No. 120, SEQ ID No. 121, SEQ ID No. 122, SEQ ID No. 123, SEQ ID No. 124, SEQ ID No. 125, SEQ ID No. 126, SEQ ID No. 127, SEQ ID No. 128, SEQ ID No. 129, SEQ ID No. 130, SEQ ID No. 131, SEQ ID No. 132, SEQ ID No. 133, SEQ ID No. 134, SEQ ID No. 135, SEQ ID No. 136, SEQ ID No. 137, SEQ ID No. 138, SEQ ID No. 139, SEQ ID No. 140, SEQ ID No. 141, SEQ ID No. 142, preferably 91%, 92%, 93%, 94%, 95%, 96%, 97%, 98%, 99% or 100% identical.

In another embodiment, the siNA agent is for use in the treatment of ophthalmic diseases in non-human mammals wherein the antisense ribonucleotide sequence is at least 90% identical to the sequences of the following list: SEQ ID No. 89, SEQ ID No. 90, SEQ ID No. 93, SEQ ID No. 97, SEQ ID No. 98, SEQ ID No. 101, SEQ ID No. 105, SEQ ID No. 106, SEQ ID No. 109, SEQ ID No. 145, SEQ ID No. 146, SEQ ID No. 149, SEQ ID No. 153, SEQ ID No. 154, SEQ ID No. 157, SEQ ID No. 161, SEQ ID No. 162, SEQ ID No. 165, SEQ ID No. 176, SEQ ID No. 177, SEQ ID No. 178, SEQ ID No. 179, SEQ ID No. 180, SEQ ID No. 181, SEQ ID No. 182, 183, SEQ ID No. 184, SEQ ID No. 185, SEQ ID No. 186, SEQ ID No. 187, SEQ ID No. 188, 189, SEQ ID No. 190, SEQ ID No. 191, SEQ ID No. 192, SEQ ID No. 193, SEQ ID No. 194, SEQ ID No. 195, SEQ ID No. 196, SEQ ID No. 197, SEQ ID No. 198, SEQ ID No. 199, SEQ ID No. 200, SEQ ID No. 201, SEQ ID No. 202, SEQ ID No. 203, SEQ ID No. 204, SEQ ID No. 205, SEQ ID No. 206, SEQ ID No. 207, SEQ ID No. 208, SEQ ID No. 209, SEQ ID No. 210, SEQ ID No. 211, SEQ ID No. 212, SEQ ID No. 213, SEQ ID No. 214, SEQ ID No. 215, SEQ ID No. 216, SEQ ID No. 217, SEQ ID No. 218, SEQ ID No. 219, SEQ ID No. 220, SEQ ID No. 221, SEQ ID No. 222; SEQ ID No. 282, SEQ ID No. 283, SEQ ID No. 284, SEQ ID No. 285, SEQ ID No. 286, SEQ ID No. 287, SEQ ID No. 288, SEQ ID No. 289, SEQ ID No. 290, SEQ ID No. 291, SEQ ID No. 292, SEQ ID No. 293, SEQ ID No. 294, SEQ ID No. 295, SEQ ID No. 296, SEQ ID No. 297, SEQ ID No. 298, SEQ ID No. 299, SEQ ID No. 300, SEQ ID No. 301, SEQ ID No. 302, SEQ ID No. 303, SEQ ID No. 304, SEQ ID No. 305, SEQ ID No. 306, SEQ ID No. 307, SEQ ID No. 308, SEQ ID No. 309, SEQ ID No. 310, SEQ ID No. 311, SEQ ID No. 312, SEQ ID No. 313, SEQ ID No. 314, SEQ ID No. 315, SEQ ID No. 316, SEQ ID No. 317, SEQ ID No. 318, SEQ ID No. 319, SEQ ID No. 320, SEQ ID No. 321, SEQ ID No. 322, SEQ ID No. 323, SEQ ID No. 324, SEQ ID No. 325, SEQ ID No. 326, SEQ ID No. 327, SEQ ID No. 328, SEQ ID No. 329, SEQ ID No. 330, SEQ ID No. 331, SEQ ID No. 332, SEQ ID No. 333, SEQ ID No. 334, SEQ ID No. 335, SEQ ID No. 336, SEQ ID No. 337, SEQ ID No. 338, SEQ ID No. 339, SEQ ID No. 340, SEQ ID No. 341, SEQ ID No. 342, SEQ ID No. 343, SEQ ID No. 344, SEQ ID No. 344, SEQ ID No. 345, preferably 91%, 92%, 93%, 94%, 95%, 96%, 97%, 98%, 99% or 100% identical; and the sense ribonucleotide sequence that is complementary to the antisense ribonucleotide sequence is at least 90% identical to the sequences of the following list: SEQ ID No. 81, SEQ ID No. 82, SEQ ID No. 83, SEQ ID No. 85, SEQ ID No. 113, SEQ ID No. 114, SEQ ID No. 117, SEQ ID No. 121, SEQ ID No. 122, SEQ ID No. 125, SEQ ID No. 129, SEQ ID No. 130, SEQ ID No. 133, SEQ ID No. 137, SEQ ID No. 138, SEQ ID No. 141, SEQ ID No. 167, SEQ ID No. 168, SEQ ID No. 169, SEQ ID No. 170, SEQ ID No. 171, SEQ ID No. 172, SEQ ID No. 173, SEQ ID No. 174, SEQ ID No. 175, SEQ ID No. 223, SEQ ID No. 224, SEQ ID No. 225, SEQ ID No. 226, SEQ ID No. 227, SEQ ID No. 228, SEQ ID No. 229, SEQ ID No. 230, SEQ ID No. 231, SEQ ID No. 232, SEQ ID No. 233, SEQ ID No. 233, SEQ ID No. 234, SEQ ID No. 235, SEQ ID No. 236, SEQ ID No. 237, SEQ ID No. 238, SEQ ID No. 239, SEQ ID No. 240, SEQ ID No. 241, SEQ ID No. 242, SEQ ID No. 243, SEQ ID No. 244, SEQ ID No. 245, SEQ ID No. 246, SEQ ID No. 247, SEQ ID No. 248, SEQ ID No. 249, SEQ ID No. 250, SEQ ID No. 251, SEQ ID No. 252, SEQ ID No. 253, SEQ ID No. 254, SEQ ID No. 255, SEQ ID No. 256, SEQ ID No. 257, SEQ ID No. 258, SEQ ID No. 259, SEQ ID No. 260, SEQ ID No. 261, SEQ ID No. 262, SEQ ID No. 263, SEQ ID No. 264, SEQ ID No. 265, SEQ ID No. 266, SEQ ID No. 267, SEQ ID No. 268, SEQ ID No. 269, SEQ ID No. 270, SEQ ID No. 271, SEQ ID No. 272, SEQ ID No. 273, SEQ ID No. 274, SEQ ID No. 275, SEQ ID No. 276, SEQ ID No. 277, SEQ ID No. 278, SEQ ID No. 279, SEQ ID No. 280, SEQ ID No. 281, preferably 91%, 92%, 93%, 94%, 95%, 96%, 97%, 98%, 99% or 100% identical.

The present disclosure also relates to a vector for delivering genetic material to a cell said vector encoding the disclosed siNA agent.

An aspect of the present disclosure relates to a pharmaceutical composition comprising at least one siNA agent as disclosed, or the disclosed vector, and a pharmaceutical acceptable carrier.

In an embodiment, said pharmaceutical composition is administered by topical eye application, subconjunctival injection, intravitreal injection, retrobulbar injection, intracameral injection, subtenon injection or deposition, intravenous injection, intravenous infusion.

The present disclosure also describes a composition for inhibiting the expression of the dopamine-beta-hydroxylase gene in a cell wherein the composition comprises a siNA agent or a vector encoding the siNA agent, wherein said siNA agent comprises an antisense ribonucleotide sequence arranged to base-pair to a substantially complementary sense ribonucleotide sequence; wherein all of the nucleotides of the sense strand and all of the nucleotides of the antisense strand comprise a 2′-O-methyl modification or a 2′-fluoro modification; wherein both the sense strand and the antisense strand independently comprise at least one phosphorothioate internucleotide linkage; wherein the antisense ribonucleotide sequence is substantially complementary to a sense ribonucleotide sequence except in up to 6 nucleotides; wherein the sense strand comprises 15 to 21 nucleotides; wherein each nucleotide or each of the first 18 contiguous nucleotides in the 5′-3′ direction comprise a modification that is different from the modification of the adjacent nucleotide; and wherein the first modification in the antisense strand is a 2′-O-methyl modification.

An aspect of the present disclosure comprises a kit comprising the disclosed composition for inhibiting the expression of the dopamine-beta-hydroxylase gene in a cell.

### BRIEF DESCRIPTION OF THE DRAWINGS

The following figures provide preferred embodiments for illustrating the disclosure and should not be seen as limiting the scope of invention.

FIG. 1: Embodiment of results of human DBH mRNA expression determined by reverse transcription quantitative real-time polymerase chain reaction (RT-qPCR) in SK-N-SH cells at three different cell densities at initial seeding (50,000, 100,000 and 150,000 cells) and 24 h later treated with 10 nM of 21/21 (length of sense/antisense sequences) un-modified nucleotide anti-DBH un-modified Hs-57-148 siNA duplex (sense SEQ ID No. 31 and antisense SEQ ID No. 55) complexed with Lipofectamine RNAiMAX (0.3%), extracted 72 h after transfection and normalized against GAPDH mRNA. Significantly different from corresponding control values (****p<0.001).

FIG. 2: Embodiment of results of human DBH mRNA expression determined by reverse transcription quantitative real-time polymerase chain reaction (RT-qPCR) in SK-N-SH cells using a forward transfection protocol (transfection 24 h after initial seeding of 100,000 cells) or a reverse transfection protocol (transfection at initial seeding of 150,000 cells) with 10 nM of 21/21 (length of sense/antisense sequences) un-modified nucleotide anti-DBH un-modified Hs-57-148 siNA duplex (sense SEQ ID No. 31 and antisense SEQ ID No. 55) complexed with Lipofectamine RNAiMAX (0.3%), extracted 24 h after forward transfection and normalized against GAPDH mRNA. Significantly different from corresponding control values (****p<0.001) and forward versus reverse transfection (**p<0.01).

FIG. 3: (A) Embodiment of results of intensity of agarose gel bands, representative of the stability of 21/21 (length of sense/antisense sequences) un-modified nucleotide human anti-DBH siNA duplexes Hs-57-148 (sense SEQ ID No. 31 and antisense SEQ ID No. 55), Hs-57-233 (sense SEQ ID No. 32 and antisense SEQ ID No. 56), and Hs-57-185 (sense SEQ ID No. 33 and antisense SEQ ID No. 57) after treatment with RNase I. siNA sequences (300 ng) were treated with RNase I (0.5 units) at 37° C. during 30 minutes. siNA duplexes were loaded onto 1% agarose gel labelled with Green Safe. Electrophoresis was run for 25 min at 100 V, and gels were visualized in ChemiDOC XRS. (B) Embodiment of results of human DBH mRNA expression determined by RT-qPCR in SK-N-SH cells treated with 3 nM of 21/21 (length of sense/antisense sequences) un-modified nucleotide human anti-DBH siNA duplexes Hs-57-148 (sense SEQ ID No. 31 and antisense SEQ ID No. 55), Hs-57-233 (sense SEQ ID No. 32 and antisense SEQ ID No. 56), and Hs-57-185 (sense SEQ ID No. 33 and antisense SEQ ID No. 57) complexed with Lipofectamine RNAiMAX (0.3%), the vehicle, extracted 24 h after forward transfection and normalized against GAPDH mRNA. Significantly different from corresponding control values (*P<0.01; **P<0.05; ***P<0.001; ****P<0.0001).

FIG. 4: (A) Embodiment of results of intensity of agarose gel bands, representative of the stability of 15/21 (length of sense/antisense sequences) asymmetric and chemically modified nucleotide human anti-DBH siNA duplexes Hs-92-285 (sense SEQ ID No. 79 and antisense SEQ ID No. 103), Hs-92-288 (sense SEQ ID No. 80 and antisense SEQ ID No. 104), and Hs-92-245 (sense SEQ ID No. 81 and antisense SEQ ID No. 105) after treatment with RNase I. siNA sequences (300 ng) were treated with RNase I (0.5 units) at 37° C. during 30 minutes. siNA duplexes were loaded onto 1% agarose gel labelled with Green Safe. Electrophoresis was run for 25 min at 100 V, and gels were visualized in ChemiDOC XRS. (B) Embodiment of results of human DBH mRNA expression determined by RT-qPCR in SK-N-SH cells treated with 3 nM of 15/21 (length of sense/antisense sequences) asymmetric and chemically modified nucleotide human anti-DBH siNA duplexes Hs-92-285 (sense SEQ ID No. 79 and antisense SEQ ID No. 103), H2-92-288 (sense SEQ ID No. 80 and antisense SEQ ID No. 104), and Hs-92-245 (sense SEQ ID No. 81 and antisense SEQ ID No. 105) complexed with Lipofectamine RNAiMAX (0.3%), the vehicle, extracted 24 h after transfection and normalized against GAPDH mRNA. Significantly different from corresponding control values (*P<0.01; **P<0.05; ***P<0.001; ****P<0.0001).

FIG. 5: (A) Embodiment of results of human DBH mRNA expression determined by RT-qPCR in SK-N-SH cells treated with 0.1 to 10 nM of 21/21 (length of sense/antisense sequences) un-modified nucleotide human anti-DBH siNA duplex Hs-57-185 (sense SEQ ID No. 33 and antisense SEQ ID No. 57) complexed with Lipofectamine RNAiMAX (0.3%), the vehicle, extracted 24 h after forward transfection and normalized against GAPDH mRNA. (B) Embodiment of results of human DBH mRNA expression determined by RT-qPCR in SK-N-SH cells treated with 0.1 to 10 nM of 15/21 (length of sense/antisense sequences) asymmetric and chemically modified nucleotide human anti-DBH siNA duplex Hs-92-245 (sense SEQ ID No. 81 and antisense SEQ ID No. 105) complexed with Lipofectamine RNAiMAX (0.3%), the vehicle, extracted 24 h after transfection and normalized against GAPDH mRNA. Significantly different from corresponding control values.

FIG. 6: (A) Embodiment of results of human DBH representative Western Blot images of protein extracted after SK-N-SH cell treatment with 10 nM of 21/21 (length of sense/antisense sequences) un-modified nucleotide human anti-DBH siNA duplexes Hs-57-148 (sense SEQ ID No. 31 and antisense SEQ ID No. 55), Hs-57-233 (sense SEQ ID No. 32 and antisense SEQ ID No. 56), and Hs-57-185 (sense SEQ ID No. 33 and antisense SEQ ID No. 57) complexed with Lipofectamine RNAiMAX (0.4%), the vehicle. Protein samples (20 μg) were loaded onto 10% SDS-PAGE gels, electrophoresis was run for 2 h at 140 V, and gel were electrotransfered to pure nitrocellulose membranes (1 h; semi-dry transfer). Membranes were incubated with the respective primary antibodies for DBH (1:1,000) and GAPDH (1:10,000) and secondary anti-rabbit and anti-mouse ALEXA antibodies, respectively. (B) Embodiment of mean results of human DBH protein expression after SK-N-SH cell treatment with 10 nM of 21/21 (length of sense/antisense sequences) un-modified nucleotide human anti-DBH siNA duplexes Hs-57-148 (sense SEQ ID No. 31 and antisense SEQ ID No. 55), H2-57-233 (sense SEQ ID No. 32 and antisense SEQ ID No. 56), and Hs-57-185 (sense SEQ ID No. 33 and antisense SEQ ID No. 57) complexed with Lipofectamine RNAiMAX (0.4%), the vehicle. Protein samples (20 μg) were loaded onto 10% SDS-PAGE gels, electrophoresis was run for 2 h at 140 V, and gel were electrotransfered to pure nitrocellulose membranes (1 h; semi-dry transfer). Membranes were incubated with the respective primary antibodies for DBH (1:1,000) and GAPDH (1:10,000) and secondary anti-rabbit and anti-mouse ALEXA antibodies, respectively. (C) Embodiment of mean results of human DBH protein expression after SK-N-SH cell treatment with 10 nM of 15/21 (length of sense/antisense sequences) asymmetric and chemically modified nucleotide human anti-DBH siNA duplexes Hs-92-288 (sense SEQ ID No. 80 and antisense SEQ ID No. 104) and Hs-92-245 (sense SEQ ID No. 81 and antisense SEQ ID No. 105) complexed with Lipofectamine RNAiMAX (0.4%), the vehicle. Protein samples (20 μg) were loaded onto 10% SDS-PAGE gels, electrophoresis was run for 2 h at 140 V, and gel were electrotransfered to pure nitrocellulose membranes (1 h; semi-dry transfer). Membranes were incubated with the respective primary antibodies for DBH (1:1,000) and GAPDH (1:10,000) and secondary anti-rabbit and anti-mouse ALEXA antibodies, respectively. Significantly different from corresponding values (*P<0.01; **P<0.05; ***P<0.001; ****P<0.0001).

FIG. 7: Embodiment of results of human DBH activity, as measured by the formation of noradrenaline after incubation with 3 μM levodopa and 1 mM ascorbic acid for 3 h, in SK-N-SH cells treated with 25 nM of 21/21 (length of sense/antisense sequences) un-modified nucleotide human anti-DBH siNA duplexes Hs-57-148 (sense SEQ ID No. 31 and antisense SEQ ID No. 55), H2-57-233 (sense SEQ ID No. 32 and antisense SEQ ID No. 56), and Hs-57-185 (sense SEQ ID No. 33 and antisense SEQ ID No. 57) complexed with Lipofectamine RNAiMAX (0.3%), the vehicle, after 72 h forward transfection. Significantly different from corresponding control values (*P<0.01; **P<0.05; ***P<0.001; ****P<0.0001).

FIG. 8: Embodiment of results of human DBH activity, as measured by the formation of noradrenaline after incubation with 3 μM levodopa and 1 mM ascorbic acid for 3 h, in SK-N-SH cells treated with 25 nM of 21/21 (length of sense/antisense sequences) un-modified nucleotide human anti-DBH siNA duplex Hs-57-185 (sense SEQ ID No. 33 and antisense SEQ ID No. 57) complexed with Lipofectamine RNAiMAX (0.3%), the vehicle, after 72 h forward transfection, in 3 consecutive weeks, each consisting in continuous exposure to 25 nM of 21/21 (length of sense/antisense sequences) un-modified nucleotide anti-DBH siNA duplex Hs-57-185 (sense SEQ ID No. 33 and antisense SEQ ID No. 57) complexed with Lipofectamine RNAiMAX (0.3%), the vehicle. Significantly different from corresponding control values (*P<0.01; **P<0.05; ***P<0.001; ****P<0.0001).

FIG. 9: Embodiment of results of human DBH activity, as measured by the formation of noradrenaline after incubation with 3 μM levodopa and 1 mM ascorbic acid for 3 h, in SK-N-SH cells treated with 25 nM of 21/21 (length of sense/antisense sequences) un-modified nucleotide human anti-DBH siNA duplex Hs-57-185 (sense SEQ ID No. 33 and antisense SEQ ID No. 57) and with the 15/21 (length of sense/antisense sequences) asymmetric and chemically modified nucleotide human anti-DBH siNA duplex Hs-92-245 (sense SEQ ID No. 81 and antisense SEQ ID No. 105) complexed with Lipofectamine RNAiMAX (0.3%), the vehicle, after 72 h forward transfection. Significantly different from corresponding control values (*P<0.01; **P<0.05; ***P<0.001; ****P<0.0001).

FIG. 10: (A) Embodiment of results of SK-N-SH cell proliferation, calcein cell fluorescence, as percentage of the average of transfection agent (Lipofectamine RNAiMAX, 0.3%). Cell proliferation was evaluated in untreated and after treatment with 25 nM 21/21 (length of sense/antisense sequences) un-modified nucleotide human anti-DBH siNA duplexes Hs-57-148 (sense SEQ ID No. 31 and antisense SEQ ID No. 55), H2-57-233 (sense SEQ ID No. 32 and antisense SEQ ID No. 56), and Hs-57-185 (sense SEQ ID No. 33 and antisense SEQ ID No. 57) or 15/21 (length of sense/antisense sequences) asymmetric and chemically modified nucleotide human anti-DBH siNA duplexes Hs-92-285 (sense SEQ ID No. 79 and antisense SEQ ID No. 103), Hs-92-288 (sense SEQ ID No. 80 and antisense SEQ ID No. 104), and Hs-92-245 (sense SEQ ID No. 81 and antisense SEQ ID No. 105) complexed with Lipofectamine RNAiMAX (0.3%), the vehicle, after 72 h forward transfection. (B) Embodiment of results of SK-N-SH cell cytotoxicity, release of LDH to the cell supernatant, as percentage of the average of transfection agent (Lipofectamine RNAiMAX, 0.3%). Cell toxicity was evaluated in untreated and after treatment with 25 nM 21/21 (length of sense/antisense sequences) un-modified nucleotide human anti-DBH siNA duplexes Hs-57-148 (sense SEQ ID No. 31 and antisense SEQ ID No. 55), H2-57-233 (sense SEQ ID No. 32 and antisense SEQ ID No. 56), and Hs-57-185 (sense SEQ ID No. 33 and antisense SEQ ID No. 57) or 15/21 (length of sense/antisense sequences) asymmetric and chemically modified nucleotide human anti-DBH siNA duplexes Hs-92-285 (sense SEQ ID No. 79 and antisense SEQ ID No. 103), Hs-92-288 (sense SEQ ID No. 80 and antisense SEQ ID No. 104), and Hs-92-245 (sense SEQ ID No. 81 and antisense SEQ ID No. 105) complexed with Lipofectamine RNAiMAX (0.3%), the vehicle, after 72 h forward transfection. Significantly different from corresponding control values (*P<0.01; **P<0.05; ***P<0.001; ****P<0.0001).

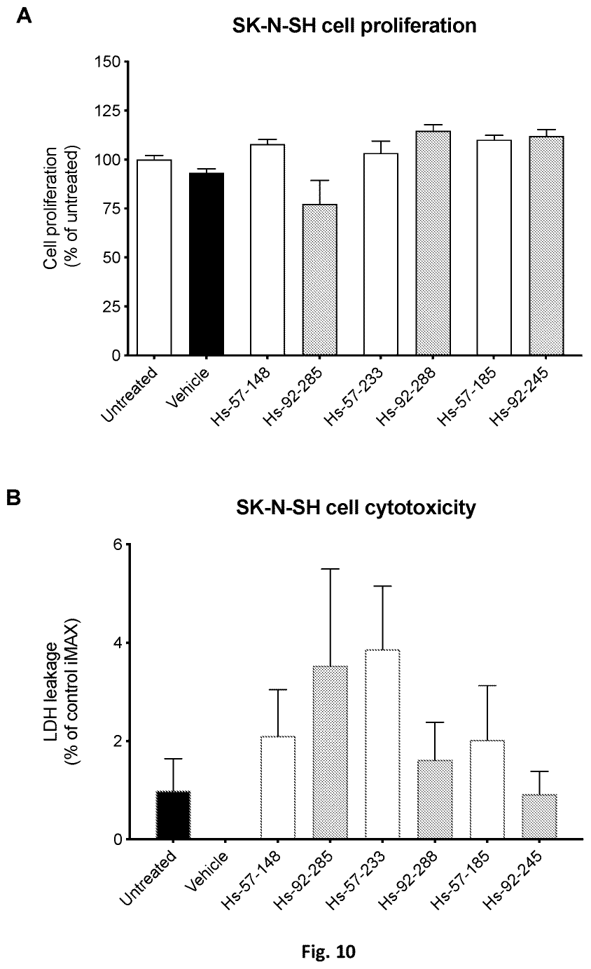

FIG. 11: (A) Embodiment of results of intensity of agarose gel bands, representative of the stability of 21/21 (length of sense/antisense sequences) un-modified nucleotide rat anti-DBH siNA duplex Rn-57-185 (sense SEQ ID No. 224 and antisense SEQ ID No. 299) and 15/21 (length of sense/antisense sequences) asymmetric and chemically modified nucleotide rat anti-DBH siNA duplex Rn-92-245 (sense SEQ ID No. 167 and antisense SEQ ID No. 208) after treatment with RNase I. siNA sequences (300 ng) were treated with RNase I (0.5 units) at 37° C. during 30 minutes. siNA duplexes were loaded onto 1% agarose gel labelled with Green Safe. Electrophoresis was run for 25 min at 100 V, and gels were visualized in ChemiDOC XRS. (B) Embodiment of results of rat DBH mRNA expression determined by RT-qPCR in PC-12 cells treated with 3 nM of 21/21 (length of sense/antisense sequences) un-modified nucleotide rat anti-DBH siNA duplex Rn-57-185 (sense SEQ ID No. 224 and antisense SEQ ID No. 299) and 15/21 (length of sense/antisense sequences) asymmetric and chemically modified nucleotide rat anti-DBH siNA duplex Rn-92-245 (sense SEQ ID No. 167 and antisense SEQ ID No. 208) complexed with Lipofectamine RNAiMAX (0.3%), the vehicle, extracted 24 h after forward transfection and normalized against GAPDH mRNA. Significantly different from corresponding control values (*P<0.01; **P<0.05; ***P<0.001; ****P<0.0001).

FIG. 12: Embodiment of results of rat DBH activity, as measured by the formation of noradrenaline after incubation with 3 μM levodopa and 1 mM ascorbic acid for 3 h, in PC-12 cells treated with 25 nM of 21/21 (length of sense/antisense sequences) un-modified nucleotide rat anti-DBH siNA duplex Rn-57-185 (sense SEQ ID No. 224 and antisense SEQ ID No. 299) and with the 21/21 (length of sense/antisense sequences) symmetric and chemically modified nucleotide rat anti-DBH siNA duplexes Rn-85-173 (sense SEQ ID No. 253 and antisense SEQ ID No. 298) and Rn-87-193 (sense SEQ ID No. 268 and antisense SEQ ID No. 313) complexed with Lipofectamine RNAiMAX (0.3%), the vehicle, after 72 h forward transfection. Significantly different from corresponding control values (*P<0.01; **P<0.05; ***P<0.001; ****P<0.0001).

FIG. 13: Embodiment of results of rat DBH activity, as measured by the formation of noradrenaline after incubation with 3 μM levodopa and 1 mM ascorbic acid for 3 h, in PC-12 cells treated with 25 nM of 21/21 (length of sense/antisense sequences) un-modified nucleotide rat anti-DBH siNA duplex Rn-57-185 (sense SEQ ID No. 224 and antisense SEQ ID No. 299) and with the 15/21 (length of sense/antisense sequences) asymmetric and chemically modified nucleotide rat anti-DBH siNA duplex Rn-92-245 (sense SEQ ID No. 167 and antisense SEQ ID No. 208) complexed with Lipofectamine RNAiMAX (0.3%), the vehicle, after 72 h forward transfection. Significantly different from corresponding control values (*P<0.01; **P<0.05; ***P<0.001; ****P<0.0001).

FIG. 14: Embodiment of results of increases in intraocular pressure (IOP) induced by eye topical application of tyramine (0.1%) in conscious female Wistar rats in (A) control conditions, (B) after topical timolol eyedrops (0.5%, 30-min before tyramine), and (C) animals pre-treated with systemic reserpine (1 mg/kg, 24 h before eye topical application of tyramine). Eyedrops containing the test materials or the vehicle (phosphate buffer saline, PBS) were applied twice (12 h apart) in the day before the first tyramine (0.1%, ocular application) test to right and left eyes, respectively. IOP measurements were made twice daily before tyramine test and then hourly for a period of 6 hours. Twelve to eight rats were used per condition. Statistical significance was determined using 2way ANOVA with Sidak's multiple comparisons test (*P<0.05; **P<0.01; ***P<0.001; ****P<0.0001).

FIG. 15: Embodiment of results of increases in intraocular pressure (IOP) induced by eye topical application of tyramine (0.1%) in conscious female Wistar rats. Eyedrops containing the (A) 15/21 (length of sense/antisense sequences) asymmetric and chemically modified nucleotide rat anti-DBH siNA duplex Rn-92-245 (sense SEQ ID No. 167 and antisense SEQ ID No. 208) or the at 4.0, 0.04 and 0.004 μg/10 μL or the vehicle (PBS) were applied twice (12 h apart) in the day before the first tyramine (0.1%, ocular application) test to right and left eyes, respectively. IOP measurements were made twice daily before tyramine test and then hourly for a period of 6 hours. Tyramine tests were performed at 24 h (A, D, G), 72 h (B, E, H) and/or 1 week (C, F) after the initial rat anti-DBH siNA duplex Rn-92-245 boost. Four to eight rats were used per condition. Statistical significance was determined using 2way ANOVA with Sidak's multiple comparisons test (*P<0.05; **P<0.01; ***P<0.001; ****P<0.0001).

FIG. 16: Embodiment of results of increases in intraocular pressure (IOP) induced by eye topical application of tyramine (0.1%) in conscious female Wistar rats. Eyedrops containing the (A) 15/21 (length of sense/antisense sequences) asymmetric and chemically modified nucleotide rat anti-DBH siNA duplex Rn-92-245 (sense SEQ ID No. 167 and antisense SEQ ID No. 208) or the (B) the 21/21 (length of sense/antisense sequences) symmetric and chemically modified nucleotide rat anti-DBH siNA duplex Rn-87-193 (sense SEQ ID No. 268 and antisense SEQ ID No. 313) at 0.04 μg/10 μL or the vehicle (PBS) were applied twice (12 h apart) in the day before the first tyramine (0.1%, ocular application) test to right and left eyes, respectively. IOP measurements were made twice daily before tyramine test and then hourly for a period of 6 hours. Tyramine tests were performed at 24 h after the initial rat anti-DBH siNA duplex Rn-92-245 or Rn-87-193 boost. Four rats were used per condition. Statistical significance was determined using 2way ANOVA with Sidak's multiple comparisons test (*P<0.05; **P<0.01; ***P<0.001; ****P<0.0001).

### DETAILED DESCRIPTION

The present disclosure relates to a siNA agent for use in the treatment of ophthalmic diseases. The siNA agent allows the targeting of the DBH gene by knocking down or inhibiting its expression as a novel strategy for ophthalmic diseases therapy. In particular, and according to a first aspect of the present disclosure, there is provided the use of a siNA for inhibiting DBH gene expression in the manufacture of a medicament for treating or preventing excessive eye sympathetic noradrenergic overactivity, wherein the siNA comprises a sense DBH nucleic acid and an antisense DBH nucleic acid. The present disclosure also describes a vector encoding the described siNA for inhibiting DBH gene expression in the manufacture of a medicament for treating or preventing ophthalmic diseases associated with the elevation of IPO due to excessive noradrenergic activation.

According to a second aspect of the present invention, there is provided a method of treating or preventing excessive eye sympathetic noradrenergic overactivity comprising administering to an individual an effective amount of a siRNA that inhibits DBH gene expression, wherein the siNA comprises a sense DBH nucleic acid and an antisense DBH nucleic acid. The present invention also provides a method of treating or preventing ophthalmic diseases associated with the elevation of IPO due to excessive noradrenergic activation comprising administering to an individual an effective amount of a vector encoding the siNA that inhibits DBH gene expression.

In an embodiment, overexpression of the DBH enzyme is a highly prevalent observation in various forms of ophthalmic diseases. The present disclosure is based on the surprising discovery that small interfering NAs (siNAs) selective for DBH are effective for treating or preventing ophthalmic diseases associated with the elevation of IPO due to excessive noradrenergic activation. In particular, diabetic retinopathy, infections, inflammation, uveitis and glaucoma, such as open-angle glaucoma, close-angle glaucoma, normal pressure glaucoma, congenital glaucoma, secondary glaucoma, pigmentary glaucoma, pseudoexfoliative glaucoma, traumatic glaucoma, neovascular glaucoma, endothelial iridocorneal syndrome and uveitic glaucoma.

In an embodiment, the siNA or vector encoding the siNA, or the pharmaceutical composition comprising the siNA or vector encoding the siNA, may be administered to an individual by topical application, nasal application, inhalation administration, subcutaneous injection or deposition, subcutaneous infusion, intravenous injection, intravenous infusion.

According to a third aspect of the present disclosure there is provided an in vitro method of inhibiting the expression of the DBH gene in a cell comprising contacting the cell with siNA that inhibits DBH gene expression as described herein. In one embodiment, said siRNA comprises a sense DBH nucleic acid and an anti-sense DBH nucleic acid, wherein the sense DBH nucleic acid is substantially identical to a target sequence contained within DBH mRNA and the anti-sense DBH nucleic acid is complementary to the sense DBH nucleic acid. The present disclosure also provides an in vitro method of inhibiting the expression of the DBH gene in a cell comprising contacting the cell with a vector encoding a siRNA that inhibits DBH gene expression, said siRNA comprises a sense DBH nucleic acid and an anti-sense DBH nucleic acid, wherein the sense DBH nucleic acid is substantially identical to a target sequence contained within DBH mRNA and the anti-sense DBH nucleic acid is complementary to the sense DBH nucleic acid.

In an embodiment, expression of the gene may be inhibited by introduction of a double stranded ribonucleic acid (dsRNA) molecule into the cell in an amount sufficient to inhibit expression of the DBH gene. In an embodiment, the siNAs of the present disclosure can cause the RNAi-mediated degradation of DBH mRNA so that the protein product of the DBH gene is not produced or is produced in reduced amounts. The disclosed siNAs can be used to alter gene expression in a cell in which expression of DBH is upregulated, e.g., as a result of excessive eye sympathetic noradrenergic overactivity. Binding of the siNA to a DBH mRNA transcript in a cell results in a reduction in DBH production by the cell.

In order that the present disclosure may be more readily understood, certain terms are first defined. In addition, it should be noted that whenever a value or range of values of a parameter are recited, it is intended that values and ranges intermediate to the recited values are also intended to be part of this invention. The articles “a” and “an” are used herein to refer to one or to more than one (i.e., to at least one) of the grammatical object of the article. By way of example, “an element” means one element or more than one element, e.g., a plurality of elements. The term “including” is used herein to mean, and is used interchangeably with, the phrase “including but not limited to”. The term “or” is used herein to mean, and is used interchangeably with, the term “and/or,” unless context clearly indicates otherwise. For example, “sense strand or antisense strand” is understood as “sense strand or antisense strand or sense strand and antisense strand.” The term “about” is used herein to mean within the typical ranges of tolerances in the art. For example, “about” can be understood as about 2 standard deviations from the mean. In certain embodiments, about means ±10%. In certain embodiments, about means ±5%. When about is present before a series of numbers or a range, it is understood that “about” can modify each of the numbers in the series or range. The term “at least” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 15 nucleotides of a 21 nucleotide nucleic acid molecule” means that 15, 16, 17, 18, 19, 20, or 21 nucleotides have the indicated property. When at least is present before a series of numbers or a range, it is understood that “at least” can modify each of the numbers in the series or range.

The term “siNA” is used to mean a double stranded RNA molecule which prevents translation of a target mRNA. Standard techniques of introducing siNA into the cell are used, including those in which DNA is a template from which RNA is transcribed. The siNA that inhibits DBH gene expression includes a sense DBH nucleic acid sequence and an antisense DBH nucleic acid sequence. The siRNA may be constructed such that a single transcript has both the sense and complementary antisense sequences from the target gene, e.g., in the form of a hairpin.

The siNA preferably comprises short double-stranded RNA that is targeted to the target mRNA, i.e., DBH mRNA. The siRNA comprises a sense RNA strand and a complementary antisense RNA strand annealed together by standard Watson-Crick base-pairing interactions (hereinafter “base-paired”). The sense strand comprises a nucleic acid sequence which is substantially identical to a target sequence contained within the DBH mRNA.

The terms “sense/antisense sequences” and “sense/antisense strands” are used interchangeable herein to refer to the parts of the siNA of the present invention that are substantially identical (sense) to the target DBH mRNA sequence or substantially complementary (antisense) to the target DBH mRNA sequence.

As used herein, a nucleic acid sequence “substantially identical” to a target sequence contained within the target mRNA is a nucleic acid sequence which is identical to the target sequence, or which differs from the target sequence by one or more nucleotides. Preferably, the substantially identical sequence is identical to the target sequence or differs from the target sequence by one, two or three nucleotides, more preferably by one or two nucleotides and most preferably by only 1 nucleotide. Sense strands which comprise nucleic acid sequences substantially identical to a target sequence are characterized in that siNA comprising such a sense strand induces RNA interference-mediated degradation of mRNA containing the target sequence. For example, an siNA of the invention can comprise a sense strand comprising a nucleic acid sequence which differs from a target sequence by one, two, three or more nucleotides, as long as RNA interference-mediated degradation of the target mRNA is induced by the siNA.

As used herein, “no more than” or “less than” is understood as the value adjacent to the phrase and logical lower values or integers, as logical from context, to zero. For example, a duplex with an overhang of “no more than 2 nucleotides” has a 2, 1, or 0 nucleotide overhang. When “no more than” is present before a series of numbers or a range, it is understood that “no more than” can modify each of the numbers in the series or range. As used herein, ranges include both the upper and lower limit.

In an embodiment, the sense and antisense strands of the siNA can comprise two complementary, single-stranded RNA molecules or can comprise a single molecule in which two complementary portions are base-paired and are covalently linked by a single-stranded “hairpin” area. That is, the sense region and antisense region can be covalently connected via a linker molecule. The linker molecule can be a polynucleotide or non-nucleotide linker. The siNA can also contain alterations, substitutions or modifications of one or more ribonucleotide bases. For example, the present siRNA can be altered, (meaning, it comprises deoxythymidine dinucleotide (dTdT)) or modified to contain one or more, preferably 0, 1, 2 or 3, deoxyribonucleotide bases. Preferably, the siRNA does not contain any deoxyribonucleotide bases.

In an embodiment, the siNA can comprise partially purified RNA, substantially pure RNA, synthetic RNA, or recombinantly produced RNA, as well as altered RNA that differs from naturally-occurring RNA by the addition, deletion, substitution and/or alteration of one or more nucleotides. Such alterations can include addition of non-nucleotide material, such as to the end(s) of the siNA or to one or more internal nucleotides of the siNA; modifications that make the siNA resistant to nuclease digestion (e.g., the use of 2′-substituted ribonucleotides or modifications to the sugar-phosphate backbone); or the substitution of one or more, preferably 0, 1, 2 or 3, nucleotides in the siNA with deoxyribonucleotides.

In an embodiment, degradation can be delayed or avoided by a wide variety of chemical modifications that include alterations in the nucleobases, sugars and the phosphate ester backbone of the siNAs. All of these chemically modified siNAs are still able to induce siNA-mediated gene silencing provided that the modifications were absent in specific regions of the siNA and included to a limited extent. In general, backbone modifications cause a small loss in binding affinity, but offer nuclease resistance. Phosphorothioate (PS)- or boranophosphate (BS)-modified siRNAs have substantial nuclease resistance. Silencing by siRNA duplexes is also compatible with some types of 2′-sugar modifications: 2′-H, 2′-O-methyl, 2′-O-methoxyethyl, 2′-fluoro (2′-F), locked nucleic acid (LNA) and ethylene-bridge nucleic acid (ENA). Suitable chemical modifications are well known to those skilled in the art. Surprisingly, the pattern of the chemical modifications used to modify the backbone of the siNA sequence is highly relevant for the stability and bioactivity of the modified sequences.

In an embodiment, the disclosed siNA described in the present disclosure is a double-stranded molecule comprising a sense strand and an antisense strand, wherein the sense strand comprises or consists of a ribonucleotide sequence corresponding to a DBH target sequence, and wherein the antisense strand comprises a ribonucleotide sequence which is complementary to said sense strand, wherein said sense strand and said antisense strand hybridize to each other to form said double-stranded molecule, and wherein said double-stranded molecule, when introduced into a cell expressing the DBH gene, inhibits expression of said gene. Said DBH target sequence preferably comprises at least about 10 contiguous, more preferably 21 to 21, and most preferably about 15 to 21 contiguous nucleotides selected from the group consisting of from sense SEQ ID No. 81/antisense SEQ ID No. 89, sense SEQ ID No. 82/antisense SEQ ID No. 90, sense SEQ ID No. 83/antisense SEQ ID No. 91, sense SEQ ID No. 85/antisense SEQ ID No. 93, sense SEQ ID No. 81/antisense SEQ ID No. 97, sense SEQ ID No. 82/antisense SEQ ID No. 98, sense SEQ ID No. 83/antisense SEQ ID No. 99, sense SEQ ID No. 85/antisense SEQ ID No. 101, sense SEQ ID No. 81/antisense SEQ ID No. 105, sense SEQ ID No. 82/antisense SEQ ID No. 106, sense SEQ ID No. 83/antisense SEQ ID No. 107, sense SEQ ID No. 85/antisense SEQ ID No. 109, sense SEQ ID No. 121/antisense SEQ ID No. 145, sense SEQ ID No. 122/antisense SEQ ID No. 146, sense SEQ ID No. 123/antisense SEQ ID No. 147, sense SEQ ID No. 125/antisense SEQ ID No. 149, sense SEQ ID No. 129/antisense SEQ ID No. 153, sense SEQ ID No. 130/antisense SEQ ID No. 154, sense SEQ ID No. 131/antisense SEQ ID No. 155, sense SEQ ID No. 133/antisense SEQ ID No. 157, sense SEQ ID No. 137/antisense SEQ ID No. 161, sense SEQ ID No. 138/antisense SEQ ID No. 162, sense SEQ ID No. 139/antisense SEQ ID No. 163, sense SEQ ID No. 141/antisense SEQ ID No. 165.

In an embodiment, the siNA disclosed in the present document can be obtained using a number of techniques known to those of skill in the art. For example, the siNA can be chemically synthesized or recombinantly produced using methods known in the art, such as the Drosophila in vitro system described in U.S. published application 2002/0086356, the entire disclosure of which is herein incorporated by reference. The siNA may be chemically synthesized using appropriately protected ribonucleoside phosphoramidites and a conventional DNA/RNA synthesizer. The siNA can be synthesized as two separate, complementary RNA molecules, or as a single RNA molecule with two complementary regions. Commercial suppliers of synthetic RNA molecules or synthesis reagents include Biospring (Frankfurt, Germany), Proligo (Hamburg, Germany), Dharmacon Research (Lafayette, Colo., USA), Thermo Fisher Scientific (Waltham, MA USA), Glen Research (Sterling, Va., USA), ChemGenes (Ashland, Mass., USA) and Sigma-Aldrich (St. Louis, MO USA).

In an embodiment, the siNA expressed from a vector can either be isolated from cultured cell expression systems by standard techniques, or can be expressed intracellularly. The vector can be used to deliver the siNA to cells in vivo, e.g., by intracellularly expressing the siNA in vivo. siNA can be expressed from a vector either as two separate, complementary RNA molecules, or as a single RNA molecule with two complementary regions. Selection of vectors suitable for expressing the siNA, methods for inserting nucleic acid sequences for expressing the siNA into the vector, and methods of delivering the vector to the cells of interest are well known to those skilled in the art.

In another embodiment, the siNA can also be expressed from a vector intracellularly in vivo. For the scope and interpretation of the present disclosure, the term “vector” means any nucleic acid- and/or viral-based technique used to deliver a desired nucleic acid. Any vector capable of accepting the coding sequences for the siRNA molecule(s) to be expressed can be used, including plasmids, cosmids, naked DNA, optionally condensed with a condensing agent, and viral vectors. Suitable viral vectors include vectors derived from adenovirus (AV); adeno-associated virus (AAV); retroviruses (e.g., lentiviruses (LV), Rhabdoviruses, murine leukemia virus); herpes virus, and the like. The tropism of viral vectors can be modified by pseudotyping the vectors with envelope proteins or other surface antigens from other viruses, or by substituting different viral capsid proteins, as appropriate. When the vector is a lentiviral vector it is preferably pseudotyped with surface proteins from vesicular stomatitis virus, rabies virus, Ebola virus or Mokola virus.

In an embodiment, vectors are produced for example by cloning a DBH target sequence into an expression vector so that operatively-linked regulatory sequences flank the DBH sequence in a manner that allows for expression (by transcription of the DNA molecule) of both strands (Lee et al., 2002). In an embodiment, an RNA molecule that is antisense to DBH mRNA is transcribed by a first promoter (e.g., a promoter sequence 3′ of the cloned DNA) and an RNA molecule that is the sense strand for the DBH mRNA is transcribed by a second promoter (e. g., a promoter sequence 5′ of the cloned DNA). The sense and antisense strands hybridize in vivo to generate siRNA constructs for silencing of the DBH gene. Alternatively, two vectors are utilized to create the sense and anti-sense strands of a siRNA construct. Cloned DBH can encode a construct having secondary structure, e. g., hairpins, wherein a single transcript has both the sense and complementary antisense sequences from the target gene. Such a transcript encoding a construct having secondary structure, will preferably comprises a single-stranded ribonucleotide sequence (loop sequence) linking said sense strand and said antisense strand.

In an embodiment, the siNA is preferably isolated. For the scope and interpretation of the present disclosure, “isolated” means synthetic, or altered or removed from the natural state through human intervention. For example, a siNA naturally present in a living animal is not “isolated,” but a synthetic siNA, or a siNA partially or completely separated from the coexisting materials of its natural state is “isolated.” An isolated siNA can exist in substantially purified form, or can exist in a non-native environment such as, for example, a cell into which the siNA has been delivered. By way of example, siNA which are produced inside a cell by natural processes, but which are produced from an “isolated” precursor molecule, are themselves “isolated” molecules. Thus, an isolated double stranded nucleic acid (dsNA) can be introduced into a target cell, where it is processed by the Dicer protein (or its equivalent) into isolated siNA.

For the scope and interpretation of the present disclosure, “inhibit” means that the activity of the DBH gene expression product or level of the DBH gene expression product is reduced below that observed in the absence of the disclosed siNA molecule. The inhibition with a siNA molecule preferably is significantly below that level observed in the presence of an inactive or attenuated molecule that is unable to mediate an RNAi response. Inhibition of gene expression with the siNA molecule is preferably significantly greater in the presence of the siNA molecule than in its absence. Preferably, the siNA inhibits the level of DBH gene expression by at least 10%, more preferably at least 50% and most preferably at least 75%.

Preferably the siNA molecule inhibits DBH gene expression so that growth of the cell containing the DBH gene is inhibited. By inhibiting DBH expression for is meant that the treated cell produces at a lower rate or has decreased the DBH protein that allows the prevention or reversion of progressive optical neuropathy associated with the elevation of IOP due to excessive noradrenergic activation. The DBH is measured by mRNA or protein assays known in the art.

For the scope and interpretation of the present disclosure, an “isolated nucleic acid” is a nucleic acid removed from its original environment (e. g., the natural environment if naturally occurring) and thus, synthetically altered from its natural state. In the present invention, isolated nucleic acid includes DNA, RNA, and derivatives thereof. When the isolated nucleic acid is RNA or derivatives thereof, base “t” should be replaced with “u” in the nucleotide sequences.

For the scope and interpretation of the present disclosure, the term “complementary” refers to Watson-Crick or Hoogsteen base pairing between nucleotides units of a polynucleotide, and the term “binding” means the physical or chemical interaction between two polypeptides or compounds or associated polypeptides or compounds or combinations thereof.

For the scope and interpretation of the present disclosure, the phrase “highly conserved sequence region” means a nucleotide sequence of one or more regions in a target gene does not vary significantly from one generation to the other or from one biological system to the other.

For the scope and interpretation of the present disclosure the term “complementarity” or “complementary” means that a nucleic acid can form hydrogen bond(s) with another nucleic acid sequence by either traditional Watson-Crick or other non-traditional types of interaction. In reference to the present invention, the binding free energy for a siNA molecule with its complementary sequence is sufficient to allow the relevant function of the nucleic acid to proceed, e.g., RNAi activity. For example, the degree of complementarity between the sense and antisense strand of the siNA molecule can be the same or different from the degree of complementarity between the antisense strand of the siRNA and the target RNA sequence.

In an embodiment, a percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. Preferably the term “complementarity” or “complementary” means that at least 90%, more preferably at least 95% and most preferably 100% of residues in a first nucleic acid sense can form hydrogen binds with a second nucleic acid sequence.

In an embodiment, complementary nucleic acid sequences hybridize under appropriate conditions to form stable duplexes containing few (one or two) or no mismatches. Furthermore, the sense strand and antisense strand of the siNA can form a double stranded nucleotide or hairpin loop structure by the hybridization. In a preferred embodiment, such duplexes contain no more than 1 mismatch for every 10 matches. In an especially preferred embodiment, the sense and antisense strands of the duplex are fully complementary, i.e., the duplexes contain no mismatches.

For the scope and interpretation of the present disclosure, the term “RNA” means a molecule comprising at least one ribonucleotide residue. By “ribonucleotide” is meant a nucleotide with a hydroxyl group at the 2 position of a beta-D-ribo-furanose moiety. The term includes double stranded RNA, single stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Such alterations can include addition of non-nucleotide material, such as to the end(s) of the siRNA or internally, for example at one or more nucleotides of the RNA. Nucleotides in the RNA molecules of the present disclosure can also comprise non-standard nucleotides, such as non-naturally occurring nucleotides or chemically synthesized nucleotides or deoxynucleotides. These altered RNAs can be referred to as analogues of naturally-occurring RNA. Preferably the term “RNA” consists of ribonucleotide residues only.

For the scope and interpretation of the present disclosure, the term “cell” is defined using its usual biological sense. The cell can be present in an organism, e.g., mammals such as humans, cows, sheep, apes, monkeys, swine, dogs, and cats. The cell can be eukaryotic (e.g., a mammalian cell). The cell can be of somatic or germ line origin, totipotent or pluripotent, dividing or non-dividing. The cell can also be derived from or can comprise a gamete or embryo, a stem cell, or a fully differentiated cell. Preferably the cell is a brain, colon, lung, mammary gland and metastatic cancer cell.

For the scope and interpretation of the present disclosure, the term“organism” refers to any living entity comprised of at least one cell. A living organism can be as simple as, for example, a single eukaryotic cell or as complex as a mammal, including a human being.

For the scope and interpretation of the present disclosure, the term “biological sample” refers to any sample containing polynucleotides. The sample may be a tissue or cell sample, or a body fluid containing polynucleotides (e.g., blood, mucus, lymphatic fluid, synovial fluid, cerebrospinal fluid, saliva, amniotic fluid, amniotic cord blood, urine, vaginal fluid and semen). The sample may be a homogenate, lysate, extract, cell culture or tissue culture prepared from a whole organism or a subset of its cells, tissues or component parts, or a fraction or portion thereof. Lastly, the sample may be a medium, such as a nutrient broth or gel in which an organism, or cells of an organism, have been propagated, wherein the sample contains polynucleotides.

For the scope and interpretation of the present disclosure, the term “subject” means an organism, which is a donor or recipient of explanted cells or the cells themselves. “Subject” also refers to an organism to which the nucleic acid molecules of the invention can be administered. The subject is preferably a mammal, e.g., a human, non-human primate, mouse, rat, dog, cat, horse, or cow. Most preferably the subject is a human.

For the scope and interpretation of the present disclosure, the terms “iRNA”, “RNAi agent,” “iRNA agent,”, “RNA interference agent” as used interchangeably, refer to an agent that contains RNA as that term is defined in the present disclosure, and which mediates the targeted cleavage of an RNA transcript via an RNA-induced silencing complex (RISC) pathway. RNA interference directs the sequence-specific degradation of mRNA through a process known as RNA interference (RNAi). The RNA interference modulates, e.g., inhibits, the expression of a DBH gene in a cell, e.g., a cell within a subject, such as a mammalian subject.

The present disclosure relates to methods of inhibiting DBH gene expression which causes the inhibition of excessive eye sympathetic noradrenergic overactivity. In particular, the invention provides a method for inhibiting DBH expression for preventing or reversing progressive optical neuropathy, wherein the optical neuropathy is selected from the following list: diabetic retinopathy, infections, inflammation, uveitis and glaucoma, such as open-angle glaucoma, close-angle glaucoma, normal pressure glaucoma, congenital glaucoma, secondary glaucoma, pigmentary glaucoma, pseudoexfoliative glaucoma, traumatic glaucoma, neovascular glaucoma, endothelial iridocorneal syndrome and uveitic glaucoma. The cell may be further contacted with a transfection-enhancing agent to enhance delivery of the siRNA or siRNA encoding vector to the cell. Depending on the specific method of the present invention, the cell may be provided in vitro, in vivo or ex vivo.

In one embodiment, an RNA interference agent as described in the present disclosure includes a single stranded RNA that interacts with a target RNA sequence, e.g., a DBH target mRNA sequence, to direct the cleavage of the target RNA. Without wishing to be bound by theory it is believed that long double stranded RNA introduced into cells is broken down into siNA by a Type III endonuclease known as Dicer (Sharp, 2001). Dicer, a ribonuclease-III-like enzyme, processes the dsRNA into 19-23 base pair short interfering RNAs with characteristic two base 3′ overhangs (Bernstein, Caudy, Hammond & Hannon, 2001). The siNAs are then incorporated into an RNC-induced silencing complex (RISC) where one or more helicases unwind the siNA duplex, enabling the complementary antisense strand to guide target recognition (Nykanen, Haley & Zamore, 2001). Upon binding to the appropriate target mRNA, one or more endonucleases within the RISC cleave the target to induce silencing (Elbashir, Lendeckel & Tuschl, 2001). Thus, in one aspect the present disclosure relates to a single stranded RNA (siRNA) generated within a cell and which promotes the formation of a RISC complex to effect silencing of the target gene, i.e., a DBH gene. Accordingly, the term “siNA” or “siRNA” is also used herein to refer to an RNA interference as described above.

In certain embodiments, the RNA interference agent may be a single-stranded siNA (ssNAi) that is introduced into a cell or organism to inhibit a target mRNA. Single-stranded NAi agents bind to the RISC endonuclease, Argonaute 2, which then cleaves the target mRNA. The single-stranded siNAs are generally 15-30 nucleotides and are chemically modified. The design and testing of single-stranded siNAs are described in U.S. Pat. No. 8,101,348 and in Lima et al. (Lima et al., 2012), the entire contents of each of which are hereby incorporated herein by reference. Any of the antisense nucleotide sequences described herein may be used as a single-stranded siNA as described herein or as chemically modified by the methods described in Lima et al. (Lima et al., 2012).

In certain embodiments, an RNA interference agent for use in the compositions, uses, and methods of the invention is a double stranded RNA and is referred to herein as a “double stranded RNA agent,” “double stranded RNA (dsRNA) molecule,” “dsRNA agent,” or “dsRNA”. The term “dsRNA”, refers to a complex of ribonucleic acid molecules, having a duplex structure comprising two anti-parallel and substantially complementary nucleic acid strands, referred to as having “sense” and “antisense” orientations with respect to a target RNA, i.e., a DBH gene. In some embodiments of the invention, a double stranded RNA (dsRNA) triggers the degradation of a target RNA, e.g., an mRNA, through a post-transcriptional gene-silencing mechanism referred to herein as RNA interference or RNAi

In general, the majority of nucleotides of each strand of a dsRNA molecule are ribonucleotides, but as described in detail herein, each or both strands can also include one or more non-ribonucleotides, e.g., a deoxyribonucleotide or a modified nucleotide. In addition, as used in this disclosure, an RNA interference agent may include ribonucleotides with chemical modifications; an RNA interference agent may include substantial modifications at multiple nucleotides. For the scope and interpretation of the present disclosure, the term “modified nucleotide” refers to a nucleotide having, independently, a modified sugar moiety, a modified internucleotide linkage, or modified nucleobase, or any combination thereof. Thus, the term “modified nucleotide” encompasses substitutions, additions or removal of, e.g., a functional group or atom, to internucleoside linkages, sugar moieties, or nucleobases. The modifications suitable for use in the agents of the invention include all types of modifications disclosed herein or known in the art. Any such modifications, as used in a siNA type molecule, are encompassed by “RNA interference agent” or “RNAi agent” for the purposes of this specification and claims

In certain embodiments of the present disclosure, inclusion of a deoxy-nucleotide if present within an RNAi agent can be considered to constitute a modified nucleotide.

The duplex region may be of any length that permits specific degradation of a desired target RNA through a RISC pathway, and may range from about 15 to 36 base pairs in length, e.g., about 15-30 base pairs in length, for example, about 9, 10, 11, 12, 13, 14, 15, 16, 17, 18, 19, 20, 21, 22, 23, 24, 25, 26, 27, 28, 29, 30, 31, 32, 33, 34, 35, or 36 base pairs in length, such as about 15-30, 15-29, 15-28, 15-27, 15-26, 15-25, 15-15, 15-23, 15-22, 15-21, 15-20, 15-19, 19-30, 19-29, 19-28, 19-27, 19-26, 19-25, 19-24, 19-23, 19-22, 19-21, 21-30, 21-29, 21-28, 21-27, 21-26, 21-25, 21-24, 21-23, or 21-22 base pairs in length. In certain embodiments, the duplex region is 15-21 base pairs in length, e.g., 21 base pairs in length. Ranges and lengths intermediate to the above recited ranges and lengths are also contemplated to be part of the disclosure.

The RNA strands may have the same or a different number of nucleotides. The maximum number of base pairs is the number of nucleotides in the shortest strand of the dsRNA minus any overhangs that are present in the duplex. In addition to the duplex structure, an RNAi may comprise one or more nucleotide overhangs. In one embodiment of the RNAi agent, at least one strand comprises a 3′ overhang of at least 1 nucleotide. In another embodiment, at least one strand comprises a 3′ overhang of at least 2 nucleotides, e.g., 2, 3, 4, 5, 6, 7, 9, 10, 11, 12, 13, 14, or 15 nucleotides. In other embodiments, at least one strand of the RNAi agent comprises a 5′ overhang of at least 1 nucleotide. In certain embodiments, at least one strand comprises a 5′ overhang of at least 2 nucleotides, e.g., 2, 3, 4, 5, 6, 7, 9, 10, 11, 12, 13, 14, or 15 nucleotides. In still other embodiments, both the 3′ and the 5′ end of one strand of the RNAi agent comprise an overhang of at least 1 nucleotide.

In certain embodiments, an RNA interference agent of the present disclosure is a dsRNA, each strand of which comprises 15-23 nucleotides, that interacts with a target RNA sequence, e.g., a DBH gene, to direct cleavage of the target RNA.

In some embodiments, the RNA interference agent of the present disclosure is a dsRNA of 24-36 nucleotides that interacts with a target RNA sequence, e.g., a DBH target mRNA sequence, to direct the cleavage of the target RNA.

For the scope and interpretation of the present disclosure, the term “nucleotide overhang” refers to at least one unpaired nucleotide that protrudes from the duplex structure of a double stranded RNA interference agent. For example, when a 3′-end of one strand of a dsRNA extends beyond the 5′-end of the other strand, or vice versa, there is a nucleotide overhang. A dsRNA can comprise an overhang of at least one nucleotide; alternatively, the overhang can comprise at least two nucleotides, at least three nucleotides, at least four nucleotides, at least five nucleotides or more. A nucleotide overhang can comprise or consist of a nucleotide/nucleoside analog, including a deoxynucleotide/nucleoside. The overhang(s) can be on the sense strand, the antisense strand, or any combination thereof. Furthermore, the nucleotide(s) of an overhang can be present on the 5′-end, 3′-end, or both ends of either an antisense or sense strand of a dsRNA.

“Blunt” or “blunt end” means that there are no unpaired nucleotides at that end of the double stranded RNA agent, i.e., no nucleotide overhang. A “blunt ended” double stranded RNA agent is double stranded over its entire length, i.e., no nucleotide overhang at either end of the molecule. The RNAi agents of the invention include RNAi agents with no nucleotide overhang at one end (i.e., agents with one overhang and one blunt end) or with no nucleotide overhangs at either end. Most often such a molecule will be double-stranded over its entire length

In an embodiment, the RNA interference agent may comprise at least one phosphorothioate or methylphosphonate internucleotide linkage. The phosphorothioate or methylphosphonate internucleotide linkage modification may occur on any nucleotide of the sense strand, antisense strand, or both strands in any position of the strand. For instance, the internucleotide linkage modification may occur on every nucleotide on the sense strand or antisense strand; each internucleotide linkage modification may occur in an alternating pattern on the sense strand or antisense strand; or the sense strand or antisense strand may contain both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand. In one embodiment, a double-stranded RNAi agent comprises 6-8 phosphorothioate internucleotide linkages. In some embodiments, the antisense strand comprises two phosphorothioate internucleotide linkages at the 5′-end and two phosphorothioate internucleotide linkages at the 3′-end, and the sense strand comprises at least two phosphorothioate internucleotide linkages at either the 5′-end or the 3′-end.

In some embodiments, the dsRNAi agent comprises a phosphorothioate or methylphosphonate internucleotide linkage modification in the overhang region. For example, the overhang region may contain two nucleotides having a phosphorothioate or methylphosphonate internucleotide linkage between the two nucleotides. Internucleotide linkage modifications also may be made to link the overhang nucleotides with the terminal paired nucleotides within the duplex region. For example, at least 2, 3, 4, or all the overhang nucleotides may be linked through phosphorothioate or methylphosphonate internucleotide linkage, and optionally, there may be additional phosphorothioate or methylphosphonate internucleotide linkages linking the overhang nucleotide with a paired nucleotide that is next to the overhang nucleotide. For instance, there may be at least two phosphorothioate internucleotide linkages between the terminal three nucleotides, in which two of the three nucleotides are overhang nucleotides, and the third is a paired nucleotide next to the overhang nucleotide. These terminal three nucleotides may be at the 3′-end of the antisense strand, the 3′-end of the sense strand, the 5′-end of the antisense strand, or the 5′end of the antisense strand.

In some embodiments, the 2-nucleotide overhang is at the 3′-end of the antisense strand, and there are two phosphorothioate internucleotide linkages between the terminal three nucleotides, wherein two of the three nucleotides are the overhang nucleotides, and the third nucleotide is a paired nucleotide next to the overhang nucleotide. Optionally, the dsRNAi agent may additionally have two phosphorothioate internucleotide linkages between the terminal three nucleotides at both the 5′-end of the sense strand and at the 5′-end of the antisense strand.

In a particular embodiment, the RNAi agent of the present disclosure comprises:
(a) a sense strand having:
   (i) a length of 15 nucleotides;
   (ii) 2′-F modifications at positions 1, 3, 5, 7, 9, 11, 13 and 15, and 2′-OMe modifications at positions 2, 4, 6, 8, 12 and 14 (counting from the 5′ end);
   (iii) phosphorothioate internucleotide linkages between nucleotide positions 1 to 3, and between nucleotide positions 13 to 15 (counting from the 5′ end)
   and
(b) an antisense strand having:
   (i) a length of 21 nucleotides;
   (ii) 2′-OMe modifications at positions 1, 3, 5, 9, 11, 13, 15, 17, 19 and 20, and 2′F modifications at positions 2, 4, 6, 8, 10, 12, 14, 16, 18 and 21 (counting from the 5′ end); and
   (iii) phosphorothioate internucleotide linkages between nucleotide positions 1 to 3, and between nucleotide positions 14 to 21 (counting from the 5′ end);
      wherein the dsRNA agents have a two nucleotide overhang at the 3′-end of the sense and antisense strands, and a blunt end at the 5′-end of the antisense strand.

In another embodiment, the RNAi agent of the present disclosure comprises:
(a) a sense strand having:
   (i) a length of 15 nucleotides;
   (ii) 2′-F modifications at positions 1, 3, 5, 7, 9, 11, 13 and 15, and 2′-OMe modifications at positions 2, 4, 6, 8, 12 and 14 (counting from the 5′ end);
   (iii) phosphorothioate internucleotide linkages between nucleotide positions 1 to 3, and between nucleotide positions 13 to 15 (counting from the 5′ end)
   and
(b) an antisense strand having:
   (i) a length of 21 nucleotides;
   (ii) 2′-OMe modifications at positions 1, 3, 5, 9, 11, 13, 15, 17, 20 and 21, and 2′F modifications at positions 2, 4, 6, 8, 10, 12, 14, 16, 18 and 19 (counting from the 5′ end); and
   (iii) phosphorothioate internucleotide linkages between nucleotide positions 1 to 3, and between nucleotide positions 14 to 21 (counting from the 5′ end);
      wherein the dsRNA agents have a two nucleotide overhang at the 3′-end of the sense and antisense strands, and a blunt end at the 5′-end of the antisense strand.

In another embodiment, the RNAi agent of the present disclosure comprises:
(a) a sense strand having:
   (i) a length of 15 nucleotides;
   (ii) 2′-F modifications at positions 1, 3, 5, 7, 9, 11, 13 and 15, and 2′-OMe modifications at positions 2, 4, 6, 8, 12 and 14 (counting from the 5′ end);
   phosphorothioate internucleotide linkages between nucleotide positions 1 to 3, and between nucleotide positions 13 to 15 (counting from the 5′ end)
   and
(b) an antisense strand having:
   (i) a length of 21 nucleotides;
   (ii) 2′-OMe modifications at positions 1, 3, 5, 9, 11, 13, 15, 17, 19 and 21, and 2′F modifications at positions 2, 4, 6, 8, 10, 12, 14, 16, 18 and 21 (counting from the 5′ end); and
   (iii) phosphorothioate internucleotide linkages between nucleotide positions 1 to 3, and between nucleotide positions 14 to 21 (counting from the 5′ end);
      wherein the dsRNA agents have a two nucleotide overhang at the 3′-end of the sense and antisense strands, and a blunt end at the 5′-end of the antisense strand.

In another embodiment, the RNAi agent of the present disclosure comprises:
(a) a sense strand having:
   (i) a length of 21 nucleotides;
   (ii) 2′-F modifications at positions 1, 3, 5, 7, 9, 11, 13, 15, 17, 19 and 21, and 2′-OMe modifications at positions 2, 4, 6, 8, 12, 14, 16, 18 and 20 (counting from the 5′ end);
   phosphorothioate internucleotide linkage between nucleotide position 21 and two dT nucleotide overhangs (counting from the 5′ end)
   and
(b) an antisense strand having:
   (i) a length of 21 nucleotides;
   (ii) 2′-OMe modifications at positions 1, 3, 5, 9, 11, 13, 15, 17, 19 and 21, and 2′F modifications at positions 2, 4, 6, 8, 10, 12, 14, 16, 18 and 20 (counting from the 5′ end); and
   (iii) phosphorothioate internucleotide linkage between nucleotide position 21 and two dT nucleotide overhangs (counting from the 5′ end);
      wherein the dsRNA agents have a two nucleotide dT overhang at the 3′-end of the sense and antisense strands, and a blunt end at the 5′-end of the antisense strand.

In another embodiment, the RNAi agent of the present disclosure comprises:
(a) a sense strand having:
   (i) a length of 21 nucleotides;
   (ii) 2′-F modifications at positions 1, 3, 5, 7, 9, 11, 13, 15, 17, 19 and 21, and 2′-OMe modifications at positions 2, 4, 6, 8, 12, 14, 16, 18 and 20, (counting from the 5′ end);
   (iii) phosphorothioate internucleotide linkage between nucleotide position 21 and two nucleotide 2′-OMe overhangs (counting from the 5′ end);
   and
(b) an antisense strand having:
   (i) a length of 21 nucleotides;
   (ii) 2′-OMe modifications at positions 1, 3, 5, 9, 11, 13, 15, 17, 19 and 21, and 2′F modifications at positions 2, 4, 6, 8, 10, 12, 14, 16, 18 and 20 (counting from the 5′ end); and
   (iii) phosphorothioate internucleotide linkage between nucleotide position 21 and two nucleotide 2′-OMe overhangs (counting from the 5′ end);
      wherein the dsRNA agents have a two nucleotide 2′-OMe overhang at the 3′-end of the sense and antisense strands, and a blunt end at the 5′-end of the antisense strand.

In another embodiment, the RNAi agent of the present disclosure comprises:
(a) a sense strand having:
   (i) a length of 21 nucleotides;
   (ii) 2′-F modifications at positions 5, 7, 8 and 9, and 2′-OMe modifications at positions 1, 2, 3, 4, 6, 10, 11, 12, 13, 14, 15, 16, 17, 18, 19, 20 and 21, (counting from the 5′ end);
   (iii) phosphorothioate internucleotide linkages between nucleotide positions 1 to 3, and between nucleotide position 21 and two nucleotide 2′-OMe overhangs (counting from the 5′ end);
   and
(b) an antisense strand having:
   (i) a length of 21 nucleotides;
   (ii) 2′-OMe modifications at positions 1, 3, 5, 7, 8, 9, 10, 11, 12, 13, 15, 17, 18, 19, 20 and 21, and 2′F modifications at positions 2, 4, 6, 14 and 16 (counting from the 5′ end); and
   (iii) phosphorothioate internucleotide linkages between nucleotide positions 1 to 3, and between nucleotide position 21 and two nucleotide 2′-OMe overhangs (counting from the 5′ end);
      wherein the dsRNA agents have a two nucleotide 2′-OMe overhang at the 3′-end of the sense and antisense strands, and a blunt end at the 5′-end of the antisense strand.

For the scope and interpretation of the present disclosure, the term “antisense strand” or “guide strand” refers to the strand of an RNA interference agent, e.g., a dsRNA, which includes a region that is substantially complementary to a target sequence, e.g., a DBH mRNA.

For the scope and interpretation of the present disclosure, the term “region of complementarity” refers to the region on the antisense strand that is substantially complementary to a sequence, for example a target sequence, e.g., a DBH nucleotide sequence, as defined herein. Where the region of complementarity is not fully complementary to the target sequence, the mismatches can be in the internal or terminal regions of the molecule. Generally, the most tolerated mismatches are in the terminal regions, e.g., within 5, 4, or 3 nucleotides of the 5′- or 3′-end of the RNA interference agent. In some embodiments, the double stranded RNA agent of the present disclosure includes a nucleotide mismatch in the antisense strand. In some embodiments, the antisense strand of the double stranded RNA agent of the present disclosure includes no more than 4 mismatches with the target mRNA, e.g., the antisense strand includes 4, 3, 2, 1, or 0 mismatches with the target mRNA. In some embodiments, the antisense strand double stranded RNA agent of the present disclosure includes no more than 4 mismatches with the sense strand, e.g., the antisense strand includes 4, 3, 2, 1, or 0 mismatches with the sense strand. In some embodiments, the double stranded RNA agent of the present disclosure includes a nucleotide mismatch in the sense strand. In some embodiments, the sense strand of the double stranded RNA agent of the present disclosure includes no more than 4 mismatches with the antisense strand, e.g., the sense strand includes 4, 3, 2, 1, or 0 mismatches with the antisense strand. In some embodiments, the nucleotide mismatch is, for example, within 5, 4, 3 nucleotides from the 3′-end of the RNA interference agent. In another embodiment, the nucleotide mismatch is, for example, in the 3′-terminal nucleotide of the RNA interference agent. In some embodiments, the mismatch(s) is not in the seed region.

Thus, an RNA interference agent as described in the present disclosure can contain one or more mismatches to the target sequence. In one embodiment, an RNA interference agent as described in the present disclosure contains no more than 3 mismatches (i.e., 3, 2, 1, or 0 mismatches). In one embodiment, an RNA interference agent as described in the present disclosure contains no more than 2 mismatches. In one embodiment, an RNA interference agent as described in the present disclosure contains no more than 1 mismatch. In one embodiment, an RNAi agent as described herein contains 0 mismatches. In certain embodiments, if the antisense strand of the RNA interference agent contains mismatches to the target sequence, the mismatch can optionally be restricted to be within the last 5 nucleotides from either the 5′- or 3′-end of the region of complementarity. For example, in such embodiments, for a 23 nucleotide RNAi agent, the strand which is complementary to a region of a DBH gene, generally does not contain any mismatch within the central 13 nucleotides. The methods described herein or methods known in the art can be used to determine whether an RNA interference agent containing a mismatch to a target sequence is effective in inhibiting the expression of a DBH gene. Consideration of the efficacy of RNA interference agents with mismatches in inhibiting expression of a DBH gene is important, especially if the particular region of complementarity in a DBH gene is known to have polymorphic sequence variation within the population.

For the scope and interpretation of the present disclosure, the term “sense strand” or “passenger strand” refers to the strand of an RNA interference agent that includes a region that is substantially complementary to a region of the antisense strand as that term is defined herein.

For the scope and interpretation of the present disclosure, the term “cleavage region” refers to a region that is located immediately adjacent to the cleavage site. The cleavage site is the site on the target at which cleavage occurs. In some embodiments, the cleavage region comprises three bases on either end of, and immediately adjacent to, the cleavage site. In some embodiments, the cleavage region comprises two bases on either end of, and immediately adjacent to, the cleavage site. In some embodiments, the cleavage site specifically occurs at the site bound by nucleotides 10 and 11 of the antisense strand, and the cleavage region comprises nucleotides 11, 12 and 13.

For the scope and interpretation of the present disclosure, “complementary” sequences can also include, or be formed entirely from, non-Watson-Crick base pairs or base pairs formed from non-natural and modified nucleotides, in so far as the above requirements with respect to their ability to hybridize are fulfilled. Such non-Watson-Crick base pairs include, but are not limited to, G:U Wobble or Hoogstein base pairing.

For the scope and interpretation of the present disclosure, the terms “complementary,” “fully complementary” and “substantially complementary” can be used with respect to the base matching between the sense strand and the antisense strand of a dsRNA, or between the antisense strand of a double stranded RNA agent and a target sequence, as will be understood from the context of their use.

For the scope and interpretation of the present disclosure, a polynucleotide that is “substantially complementary to at least part of” a messenger RNA (mRNA) refers to a polynucleotide that is substantially complementary to a contiguous portion of the mRNA of interest (e.g., an mRNA encoding a DBH gene). For example, a polynucleotide is complementary to at least a part of a DBH mRNA if the sequence is substantially complementary to a non-interrupted portion of an mRNA encoding a DBH gene.

Accordingly, in some embodiments, the antisense polynucleotides of the present disclosure are fully complementary to the target DBH sequence. In other embodiments, the antisense polynucleotides disclosed herein are substantially complementary to the target DBH sequence and comprise a contiguous nucleotide sequence which is at least 80% complementary over its entire length to the equivalent region of the nucleotide sequence of any one of SEQ ID Nos: 111, 112, 113, 114, 115, 116, 117, 118, 223, 224, 225, 226, 227, 228, 229, 230, 231, 232, 233, 234, 235, 236, 237 or 238, or a fragment of any one of SEQ ID Nos: 111, 112, 113, 114, 115, 116, 117, 118, 223, 224, 225, 226, 227, 228, 229, 230, 231, 232, 233, 234, 235, 236, 237 or 238, such as about 85%, about 90%, about 91%, about 92%, about 93%, about 94%, about 95%, about 96%, about 97%, about 98%, or about 99% complementary

In some embodiments, the antisense polynucleotides disclosed herein are substantially complementary to a fragment of a target human DBH sequence and comprise a contiguous nucleotide sequence which is at least 80% complementary over its entire length to a fragment of SEQ ID No. 1 selected from the group of nucleotides 279-299; 693-713; 1341-1341; 1344-1364; 1402-1422; 1560-1580; 1722-1742; 1759-1779, such as about 85%, about 90%, about 91%, about 92%, about 93%, about 94%, about 95%, about 96%, about 97%, about 98%, or about 99% complementary

In some embodiments, the antisense polynucleotides of the present disclosure are substantially complementary to a fragment of a target human DBH sequence and comprise a contiguous nucleotide sequence which is at least 80% complementary over its entire length to a fragment of SEQ ID No. 1 selected from the group of nucleotides 279-299; 693-713; 1341-1341; 1344-1364, such as about 85%, about 90%, about 91%, about 92%, about 93%, about 94%, about 95%, about 96%, about 97%, about 98%, or about 99% complementary

In other embodiments, the antisense polynucleotides of the present disclosure are substantially complementary to the target DBH sequence and comprise a contiguous nucleotide sequence which is at least about 80% complementary over its entire length to any one of the sense strand nucleotide sequences in any one of any one of Tables 3, 4, 5, 6 or 7, or a fragment of any one of the sense strand nucleotide sequences in any one of Tables 3, 4, 5, 6 or 7, such as about 85%, about 90%, about 91%, about 92%, about 93%, about 94%, about 95%, about 96%, about 97%, about 98%, about 99%, or 100% complementary

In certain embodiments, the sense and antisense strands are selected from any one of anti-DBH siNA duplexes Hs-92-195 (SEQ ID No. 81 sense/SEQ ID No. 89 antisense), Hs-92-130 (SEQ ID No. 82 sense/SEQ ID No. 90 antisense), Hs-92-267 (SEQ ID No. 83 sense/SEQ ID No. 91 antisense), Hs-92-216 (SEQ ID No. 85 sense/SEQ ID No. 93 antisense), Hs-92-286 (SEQ ID No. 81 sense/SEQ ID No. 97 antisense), Hs-92-155 (SEQ ID No. 82 sense/SEQ ID No. 98 antisense), Hs-92-174 (SEQ ID No. 83 sense/SEQ ID No. 99 antisense), Hs-92-221 (SEQ ID No. 85 sense/SEQ ID No. 101 antisense), Hs-92-245 (SEQ ID No. 81 sense/SEQ ID No. 105 antisense), Hs-92-134 (SEQ ID No. 82 sense/SEQ ID No. 106 antisense), Hs-92-225 (SEQ ID No. 83 sense/SEQ ID No. 107 antisense), Hs-92-171 (SEQ ID No. 85 sense/SEQ ID No. 109 antisense), Hs-81-253 (SEQ ID No. 121 sense/SEQ ID No. 145 antisense), Hs-81-277 (SEQ ID No. 122 sense/SEQ ID No. 146 antisense), Hs-81-244 (SEQ ID No. 123 sense/SEQ ID No. 147 antisense), Hs-81-247 (SEQ ID No. 125 sense/SEQ ID No. 149 antisense), Hs-85-173 (SEQ ID No. 129 sense/SEQ ID No. 153 antisense), Hs-85-163 (SEQ ID No. 130 sense/SEQ ID No. 154 antisense), Hs-85-231 (SEQ ID No. 131 sense/SEQ ID No. 155 antisense), Hs-85-300 (SEQ ID No. 133 sense/SEQ ID No. 157 antisense), Hs-87-193 (SEQ ID No. 137 sense/SEQ ID No. 161 antisense), Hs-87-156 (SEQ ID No. 138 sense/SEQ ID No. 162 antisense), Hs-87-230 (SEQ ID No. 139 sense/SEQ ID No. 163 antisense), Hs-87-235 (SEQ ID No. 141 sense/SEQ ID No. 165 antisense). In some embodiments, the sense and antisense strands are selected from any one of anti-DBH siNA duplexes Clf-92-195 (SEQ ID No. 81 sense/SEQ ID No. 89 antisense), Ec-92-195 (SEQ ID No. 81 sense/SEQ ID No. 89 antisense), Clf-92-130 (SEQ ID No. 82 sense/SEQ ID No. 90 antisense), Ec-92-130 (SEQ ID No. 82 sense/SEQ ID No. 90 antisense), Clf-92-267 (SEQ ID No. 170 sense/SEQ ID No. 180 antisense), Ec-92-267 (SEQ ID No. 171 sense/SEQ ID No. 181 antisense), Clf-92-216 (SEQ ID No. 172 sense/SEQ ID No. 186 antisense), Ec-92-216 (SEQ ID No. 85 sense/SEQ ID No. 187 antisense), Clf-92-286 (SEQ ID No. 81 sense/SEQ ID No. 97 antisense), Ec-92-286 (SEQ ID No. 81 sense/SEQ ID No. 97 antisense), Clf-92-155 (SEQ ID No. 82 sense/SEQ ID No. 98 antisense), Ec-92-155 (SEQ ID No. 82 sense/SEQ ID No. 98 antisense), Clf-92-174 (SEQ ID No. 170 sense/SEQ ID No. 195 antisense), Ec-92-174 (SEQ ID No. 171 sense/SEQ ID No. 196 antisense), Clf-92-221 (SEQ ID No. 172 sense/SEQ ID No. 202 antisense), Ec-92-221 (SEQ ID No. 85 sense/SEQ ID No. 203 antisense), Clf-92-245 (SEQ ID No. 81 sense/SEQ ID No. 105 antisense), Ec-92-245 (SEQ ID No. 81 sense/SEQ ID No. 105 antisense), Clf-92-134 (SEQ ID No. 82 sense/SEQ ID No. 106 antisense), Ec-92-134 (SEQ ID No. 82 sense/SEQ ID No. 106 antisense), Clf-92-225 (SEQ ID No. 170 sense/SEQ ID No. 211 antisense), Ec-92-225 (SEQ ID No. 171 sense/SEQ ID No. 212 antisense), Clf-92-171 (SEQ ID No. 172 sense/SEQ ID No. 218 antisense), Ec-92-171 (SEQ ID No. 85 sense/SEQ ID No. 219 antisense), Clf-81-253 (SEQ ID No. 121 sense/SEQ ID No. 145 antisense), Ec-81-253 (SEQ ID No. 121 sense/SEQ ID No. 145 antisense), Clf-81-277 (SEQ ID No. 122 sense/SEQ ID No. 146 antisense), Ec-81-277 (SEQ ID No. 122 sense/SEQ ID No. 146 antisense), Clf-81-244 (SEQ ID No. 242 sense/SEQ ID No. 287 antisense), Ec-81-244 (SEQ ID No. 243 sense/SEQ ID No. 288 antisense), Clf-81-247 (SEQ ID No. 248 sense/SEQ ID No. 293 antisense), Ec-81-247 (SEQ ID No. 249 sense/SEQ ID No. 294 antisense), Clf-85-173 (SEQ ID No. 129 sense/SEQ ID No. 153 antisense), Ec-85-173 (SEQ ID No. 129 sense/SEQ ID No. 153 antisense), Clf-85-163 (SEQ ID No. 130 sense/SEQ ID No. 154 antisense), Ec-85-163 (SEQ ID No. 130 sense/SEQ ID No. 154 antisense), Clf-85-231 (SEQ ID No. 257 sense/SEQ ID No. 302 antisense), Ec-85-231 (SEQ ID No. 258 sense/SEQ ID No. 303 antisense), Clf-85-300 (SEQ ID No. 263 sense/SEQ ID No. 308 antisense), Ec-85-300 (SEQ ID No. 264 sense/SEQ ID No. 309 antisense), Clf-87-193 (SEQ ID No. 137 sense/SEQ ID No. 161 antisense), Ec-87-193 (SEQ ID No. 137 sense/SEQ ID No. 161 antisense), Clf-87-156 (SEQ ID No. 138 sense/SEQ ID No. 162 antisense), Ec-87-156 (SEQ ID No. 138 sense/SEQ ID No. 162 antisense), Clf-87-230 (SEQ ID No. 272 sense/SEQ ID No. 317 antisense), Ec-87-230 (SEQ ID No. 273 sense/SEQ ID No. 318 antisense), Clf-87-235 (SEQ ID No. 278 sense/SEQ ID No. 323 antisense), Ec-87-235 (SEQ ID No. 279 sense/SEQ ID No. 324 antisense).

Sequence information regarding the human DBH gene (GenBank accession NM_000787.4) was extracted from the NCBI Entrez nucleotide database. Up to 8 mRNA segments were identified. Methods for designing double stranded RNA having the ability to inhibit gene expression in a target cell are known. See for example, U.S. Pat. No. 6,506,559, and (Elbashir, Harborth, Lendeckel, Yalcin, Weber & Tuschl, 2001), herein incorporated by reference in its entirety.

In an embodiment, selection of siRNA target sites can be performed as follows:
Beginning with the ATG start codon of the transcript, scan downstream for AA dinucleotide sequences. Record the occurrence of each AA and the 3′ adjacent 19 nucleotides as potential siRNA target sites. Tuschl et al. (Tuschl, Sharp & Bartel, 1998) recommend against designing siRNA to the 5′ and 3′ untranslated regions (UTRs) and regions near the start codon (within 75 bases) as these may be richer in regulatory protein binding sites. UTR-binding proteins and/or translation initiation complexes may interfere with binding of the siRNA endonuclease complex.
Compare the potential target sites to the appropriate genome database (human, mouse, rat, etc.) and eliminate from consideration any target sequences with significant homology to other coding sequences. In a preferred embodiment, BLAST is used, which can be found on the NCBI server at: www.ncbi.nlm.nih.gov/BLAST/
Select qualifying target sequences (i.e., sequences having over 45% GC content) for synthesis.

In one aspect of the invention, the length of the sense nucleic acid is at least 10 nucleotides and may be as long as the naturally-occurring DBH transcript.

Preferably, the sense nucleic acid is less than 75, 50, or 25 nucleotides in length. It is further preferred that the sense nucleic acid comprises at least 15 nucleotides. Most preferably, the sense nucleic acid is 15-25 nucleotides in length

Examples of DBH siRNA sense nucleic acids of the present invention which inhibit DBH expression in mammalian cells include oligonucleotides comprising any one of the following target sequences of the DBH gene: nucleotides 279-299; 693-713; 1341-1341; 1344-1364; 1402-1422; 1560-1580; 1722-1742; 1759-1779.

In an aspect of the disclosure, an agent for use in the methods and compositions of the disclosure is a single-stranded antisense oligonucleotide molecule that inhibits a target mRNA via an antisense inhibition mechanism. The single-stranded antisense oligonucleotide molecule is complementary to a sequence within the target mRNA. The single-stranded antisense oligonucleotides can inhibit translation in a stoichiometric manner by base pairing to the mRNA and physically obstructing the translation machinery (Dias & Stein, 2002). The single-stranded antisense oligonucleotide molecule may be about 14 to about 30 nucleotides in length and have a sequence that is complementary to a target sequence. For example, the single-stranded antisense oligonucleotide molecule may comprise a sequence that is at least about 14, 15, 16, 17, 18, 19, 20, or more contiguous nucleotides from any one of the antisense sequences described herein.

The phrase “contacting a cell with an RNA interference agent,” such as a dsRNA, as used herein, includes contacting a cell by any possible means. Contacting a cell with an RNA interference agent includes contacting a cell in vitro with the RNA interference agent or contacting a cell in vivo with the RNA interference agent. The contacting may be done directly or indirectly. Thus, for example, the RNA interference agent may be put into physical contact with the cell by the individual performing the method, or alternatively, the RNA interference agent may be put into a situation that will permit or cause it to subsequently come into contact with the cell.

In an embodiment, contacting a cell in vitro may be done, for example, by incubating the cell with the RNA interference agent. Contacting a cell in vivo may be done, for example, by injecting the RNA interference agent into or near the tissue where the cell is located, or by injecting the RNA interference agent into another area, e.g., the bloodstream or the subcutaneous space, such that the agent will subsequently reach the tissue where the cell to be contacted is located. For example, the RNA interference agent may contain or be coupled to a ligand that directs the RNA interference agent to a site of interest, e.g., cells with high DBH expression and activity. Combinations of in vitro and in vivo methods of contacting are also possible.

For the scope and interpretation of the present disclosure, a “subject” is an animal, such as a mammal, including a primate (such as a human, a non-human primate, e.g., a monkey, and a chimpanzee), a non-primate (such as a cow, a pig, a horse, a goat, a rabbit, a sheep, a hamster, a guinea pig, a cat, a dog, a rat, or a mouse), or a bird that expresses the target gene, either endogenously or heterologously. In an embodiment, the subject is a human, such as a human being treated or assessed for a disease or disorder that would benefit from reduction in DBH expression; a human at risk for a disease or disorder that would benefit from reduction in DBH expression; a human having a disease or disorder that would benefit from reduction in DBH expression; or human being treated for a disease or disorder that would benefit from reduction in DBH expression as described. In some embodiments, the subject is a female human. In other embodiments, the subject is a male human. In one embodiment, the subject is an adult subject. In another embodiment, the subject is a paediatric subject.

For the scope and interpretation of the present disclosure, the terms “treating” or “treatment” refer to a beneficial or desired result, such as reducing at least one sign or symptom of an excessive eye sympathetic noradrenergic overactivity in a subject. Treatment also includes a reduction of one or more sign or symptoms associated with unwanted excessive eye sympathetic noradrenergic overactivity; diminishing the extent of unwanted excessive eye sympathetic noradrenergic overactivity; amelioration or palliation of unwanted excessive eye sympathetic noradrenergic overactivity. “Treatment” can also mean preventing or reversing progressive optical neuropathy as compared to expected progressive optical neuropathy in the absence of treatment. The term “lower” in the context of the level of DBH in a subject or a disease marker or symptom refers to a statistically significant decrease in such level. The decrease can be, for example, at least 10%, 15%, 20%, 25%, 30%, %, 40%, 45%, 50%, 55%, 60%, 65%, 70%, 75%, 80%, 85%, 90%, 95%, or more. In certain embodiments, a decrease is at least 20%. In certain embodiments, the decrease is at least 50% in a disease marker, e.g., protein or gene expression level. “Lower” in the context of the level of excessive eye sympathetic noradrenergic overactivity in a subject is preferably down to a level accepted as within the range of normal for an individual without such disorder. In certain embodiments, “lower” is the decrease in the difference between the level of a marker or symptom for a subject suffering from a disease and a level accepted within the range of normal for an individual, e.g., the level of decrease in bodyweight between an obese individual and an individual having a weight accepted within the range of normal.

For the scope and interpretation of the present disclosure, the term “excessive eye sympathetic noradrenergic overactivity associated disease”, is a disease or disorder that is caused by, or associated with excessive eye sympathetic noradrenergic overactivity associated with the elevation of intraocular pressure IOP. The terms “excessive eye sympathetic noradrenergic overactivity associated disease” includes a disease, disorder or condition that would benefit from a decrease in DBH gene expression, replication, or protein activity. Non-limiting examples of excessive eye sympathetic noradrenergic overactivity associated disease include, for example, progressive optical neuropathy, wherein the optical neuropathy is selected from the following list: diabetic retinopathy, infections, inflammation, uveitis and glaucoma, such as open-angle glaucoma, close-angle glaucoma, normal pressure glaucoma, congenital glaucoma, secondary glaucoma, pigmentary glaucoma, pseudoexfoliative glaucoma, traumatic glaucoma, neovascular glaucoma, endothelial iridocorneal syndrome and uveitic glaucoma.

For the scope and interpretation of the present disclosure, “therapeutically effective amount,” as used herein, is intended to include the amount of an RNAi agent that, when administered to a subject having a DBH-associated disease, is sufficient to effect treatment of the disease (e.g., by diminishing, ameliorating, or maintaining the existing disease or one or more symptoms of disease). The “therapeutically effective amount” may vary depending on the RNAi agent, how the agent is administered, the disease and its severity and the history, age, weight, family history, genetic makeup, the types of preceding or concomitant treatments, if any, and other individual characteristics of the subject to be treated.

For the scope and interpretation of the present disclosure, “Prophylactically effective amount,” is intended to include the amount of an RNA interference agent that, when administered to a subject having a DBH-associated disorder, is sufficient to prevent or ameliorate the disease or one or more symptoms of the disease. Ameliorating the disease includes slowing the course of the disease or reducing the severity of later-developing disease. The “prophylactically effective amount” may vary depending on the RNA interference agent, how the agent is administered, the degree of risk of disease, and the history, age, weight, family history, genetic makeup, the types of preceding or concomitant treatments, if any, and other individual characteristics of the patient to be treated.

For the scope and interpretation of the present disclosure, a “therapeutically-effective amount” or “prophylactically effective amount” also includes an amount of an RNA interference agent that produces some desired effect at a reasonable benefit/risk ratio applicable to any treatment. The RNA interference agent employed in the methods of the present invention may be administered in a sufficient amount to produce a reasonable benefit/risk ratio applicable to such treatment.

For the scope and interpretation of the present disclosure, “pharmaceutically acceptable” is employed to refer to those compounds, materials, compositions, or dosage forms which are, within the scope of sound medical judgment, suitable for use in contact with the tissues of human subjects and animal subjects without excessive toxicity, irritation, allergic response, or other problem or complication, commensurate with a reasonable benefit/risk ratio.

For the scope and interpretation of the present disclosure, “pharmaceutically-acceptable carrier” means a pharmaceutically-acceptable material, composition, or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject being treated. Such carriers are known in the art. Pharmaceutically acceptable carriers include carriers for administration by injection.

The present disclosure is more particularly described in the following examples that are intended as illustrative only since numerous modifications and variations are possible and will be apparent to those skilled in the art.

In an embodiment, the siNA of the present disclosure is for use as a medicament.

In an embodiment, the siRNA of the present disclosure is for use as a medicament for preventing or reversing progressive optical neuropathy associated with the elevation of intraocular pressure due to excessive noradrenergic activation.

In an embodiment, the method for preventing or reversing progressive optical neuropathy associated with the elevation of intraocular pressure due to excessive noradrenergic activation comprises administering at least one siNA molecule, as described herein, to a patient or subject in need thereof.

In an embodiment, the optical neuropathy disorder is selected from the following list: diabetic retinopathy, infections, inflammation, uveitis and glaucoma, such as open-angle glaucoma, close-angle glaucoma, normal pressure glaucoma, congenital glaucoma, secondary glaucoma, pigmentary glaucoma, pseudoexfoliative glaucoma, traumatic glaucoma, neovascular glaucoma, endothelial iridocorneal syndrome and uveitic glaucoma.

In an embodiment, the disclosure relates to a pharmaceutical composition comprising at least one siNA of the present disclosure and a pharmaceutically acceptable carrier.

In an embodiment, the siNA of the present disclosure inhibits the in vitro expression of dopamine-beta-hydroxylase protein expression. In vitro dopamine-beta-hydroxylase protein expression is inhibited by administering a siNA of the present disclosure into a cell.

In an embodiment, the in vitro dopamine-beta-hydroxylase expression in a cell is inhibited by up to or more than 10%, 20%, 30%, 40%, 50%, 60%, 70%, 80% or 90% as compared to the level in a control.

In an embodiment, the siNA of the present disclosure inhibits the in vitro activity of dopamine-beta-hydroxylase. In vitro dopamine-beta-hydroxylase activity is inhibited by administering a siNA of the present disclosure into a cell.

In an embodiment, the dopamine-beta-hydroxylase activity is inhibited by up to or more than 10%, 20%, 30%, 40%, 50%, 60%, 70%, 80% or 90% as compared to the level in a control.

In an embodiment, the present disclosure relates to a method of reducing dopamine-beta-hydroxylase protein expression and activity, preferably in a patient, the method comprising administering at least one siNA of the present disclosure.

In an embodiment, the decrease in dopamine-beta-hydroxylase protein expression and activity may be up to or more than 10%, 20%, 30%, 40%, 50%, 60%, 70%, 80% or 90% as compared to the level in a control.

In an embodiment, the disclosure relates to methods of reducing dopamine-beta-hydroxylase protein expression and activity in a cell comprising treating the cells with an siNA of the disclosure in combination with one or more agents known in the art, preferably wherein the agent comprises an anti-glaucoma agent and most preferably alpha adrenoceptor agonists (apraclonidine, brimonidine), beta adrenoceptor blockers (betaxolol, levobunolol, metpranolol, timolol), carbonic anhydrase inhibitors (acetazolamide, brinzolamide, dorzolamide, methazolamide), muscarinic agonists (carbachol, pilocarpine), prostaglandin analogs (bimatoprost, latanoprost, tafluprost, travaprost), rho kinase inhibitors (netarsudil).

In an embodiment, the present disclosure also relates to methods of preventing or reversing progressive optical neuropathy associated with the elevation of intraocular pressure due to excessive noradrenergic activation comprising administrating an siNA of the present disclosure in combination with one or more anti-glaucoma agents known in the art, preferably to a patient in need thereof.

In an embodiment, the anti-glaucoma agent comprises an anti-glaucoma agent, more preferably an alpha adrenoceptor agonists, beta adrenoceptor blockers, carbonic anhydrase inhibitors, muscarinic agonists, prostaglandin analogs and rho kinase inhibitors agent and most preferably apraclonidine, brimonidine, betaxolol, levobunolol, metpranolol, timolol, acetazolamide, brinzolamide, dorzolamide, methazolamide, carbachol, pilocarpine, bimatoprost, latanoprost, tafluprost, travaprost, and netarsudil.

In an embodiment, the disclosure further relates to pharmaceutical compositions comprising the siNA of the present disclosure and one or more anti-glaucoma agent.

In an embodiment, the disclosure relates to methods for increasing the efficacy of an anti-glaucoma therapy given to a patient. The method comprising administering an siNA of the present disclosure in combination with the therapy.

In an embodiment, the increase in anti-glaucoma therapy efficacy may be up to or more than 10%, 20%, 30%, 40%, 50%, 60%, 70%, 80% or 90% as compared to the efficacy of either administration of siNA or the anti-glaucoma agent alone.

In an embodiment, the disclosure also relates to methods of treating glaucoma comprising administrating an siNA of the present disclosure in combination with one or more types of laser surgery known in the art to treat glaucoma, preferably to a patient in need thereof.

In an embodiment, the laser surgery comprises trabeculoplasty or iridotomy, trabeculotomy and implantation of glaucoma drainage devices.

In an embodiment, the disclosure further relates to pharmaceutical compositions comprising the siNA of the present disclosure and one or more types of laser surgery, trabeculotomy and implantation of glaucoma drainage devices.

In an embodiment, the disclosure relates to methods for increasing the efficacy of laser surgery, trabeculotomy and implantation of glaucoma drainage devices, performed in a patient comprising administering an siNA of the disclosure in combination with the therapy.

In an embodiment, the increase in laser surgery efficacy may be up to or more than 10%, 20%, 30%, 40%, 50%, 60%, 70%, 80% or 90% when compared to the efficacy of either administration of siNA or the laser surgery (trabeculoplasty or iridotomy), trabeculotomy and implantation of glaucoma drainage devices inhibition therapy alone.

### EXAMPLES

### Example 1—siNA Design

Where the source of a reagent is not specifically given herein, such reagent can be obtained from any supplier of reagents for molecular biology at a quality/purity standard for application in molecular biology.

In an embodiment, siNAs targeting the human DBH gene (human: NCBI RefSeq ID NM_000787.4; NCBI Gene ID: 1621) were designed using custom MATLAB scripts. The human NM_000787.4 RefSeq mRNA, has a length of 2745 bases. Detailed lists of the unmodified DBH sense and antisense strand nucleotide sequences are shown in Table 3. Detailed lists of the modified DBH sense and antisense strand nucleotide sequences are shown in Tables 4, 5, 6, and 7.

In another embodiment, siNAs targeting the DBH gene in the monkey (NCBI RefSeq ID NM_000787.4; NCBI Gene ID: 1621), dog (NCBI RefSeq ID NM_000787.4; NCBI Gene ID: 1621), horse (NCBI RefSeq ID NM_000787.4; NCBI Gene ID: 1621), bovine (NCBI RefSeq ID NM_000787.4; NCBI Gene ID: 1621), cat (NCBI RefSeq ID NM_000787.4; NCBI Gene ID: 1621), mouse (NCBI RefSeq ID NM_000787.4; NCBI Gene ID: 1621) and rat (NCBI RefSeq ID NM_000787.4; NCBI Gene ID: 1621) were designed using custom MATLAB scripts. The monkey, dog, horse, bovine, cat, mouse and rat DBH mRNA target sequences are mentioned in Table 8. Detailed lists of the modified DBH sense and antisense strand nucleotide sequences against mRNA DBH in monkey, dog, horse, bovine, cat, mouse and rat are shown in Table 9. Detailed lists of the un-modified DBH sense and antisense strand nucleotide sequences against mRNA DBH in monkey, dog, horse, bovine, cat, mouse and rat are shown in Table 10.

TABLE 1
Sequence information relative to SEQ ID No. 1 to SEQ ID No. 30, concerning human (Homo sapiens)
DBH protein (SEQ ID No. 1 to SEQ ID No. 3), Macaca mulatta DBH protein (SEQ ID No. 4 to SEQ
ID No. 7), Macaca fascicularis DBH protein (SEQ ID No. 7 to SEQ ID No. 12), Canis lupus familiaris
(SEQ ID No. 13 to SEQ ID No. 15), Equus caballus (SEQ ID No. 16 to SEQ ID No. 18), Bos taurus DBH
protein (SEQ ID No. 19 to SEQ ID No. 21), Felis catus DBH protein (SEQ ID No. 22 to SEQ ID No.
24), Mus musculus DBH protein (SEQ ID No. 25 to SEQ ID No. 27) and Rattus norvegicus DBH
protein (SEQ ID No. 28 to SEQ ID No. 30).

| | | | Protein | | Nucleotide |
|---|---|---|---|---|---|
| SEQ | | | GenBank | Nucleotide | GenBank |
| ID No. | Specie | Direction | Accession No | NCBI SEQ | Accession No |
| 1 | Homo sapiens | | NP_000778.3 | | |
| 2 | Homo sapiens | forward | | GI: 1653961349 | NM_000787.4 |
| 3 | Homo sapiens | reverse | | GI: 1653961349 | NM_000787.4 |
| | | complement | | | |
| 4 | Macaca mulatta | | NP_001265274.2 | | |
| 5 | Macaca mulatta | forward | | GI: 574105 | NM_001278345.2 |
| 6 | Macaca mulatta | reverse | | GI: 574105 | NM_001278345.2 |
| | | complement | | | |
| 7 | Macaca fascicularis | | XP_005580554.2 | | |
| 8 | Macaca fascicularis | forward | | GI: 2161843712 | XM_005580497.3 |
| 9 | Macaca fascicularis | reverse | | GI: 2161843712 | XM_005580497.3 |
| | | complement | | | |
| 10 | Macaca fascicularis | | XP_045228715.1 | | |
| 11 | Macaca fascicularis | forward | | GI: 2161843710 | XM_045372780.1 |
| 12 | Macaca fascicularis | reverse | | GI: 2161843710 | XM_045372780.1 |
| | | complement | | | |
| 13 | Canis lupus familiaris | | NP_001005263.1 | | |
| 14 | Canis lupus familiaris | forward | | GI: 55742739 | NM_001005263.1 |
| 15 | Canis lupus familiaris | reverse | | GI: 55742739 | NM_001005263.1 |
| | | complement | | | |
| 16 | Equus caballus | | NP_001075239.2 | | |
| 17 | Equus caballus | forward | | GI: 1409931484 | NM_001081770.3 |
| 18 | Equus caballus | reverse | | GI: 1409931484 | NM_001081770.3 |
| | | complement | | | |
| 19 | Bos taurus | | NP_851338.1 | | |
| 20 | Bos taurus | forward | | GI: 31340938 | NM_180995.2 |
| 21 | Bos taurus | reverse | | GI: 31340938 | NM_180995.2 |
| | | complement | | | |
| 22 | Felis catus | | XP_003996085.3 | | |
| 23 | Felis catus | forward | | GI: 2131045731 | XM_003996036.6 |
| 24 | Felis catus | reverse | | GI: 2131045731 | XM_003996036.6 |
| | | complement | | | |
| 25 | Mus musculus | | NP_620392.2 | | |
| 26 | Mus musculus | forward | | GI: 110815860 | NM_138942.3 |
| 27 | Mus musculus | reverse | | GI: 110815860 | NM_138942.3 |
| | | complement | | | |
| 28 | Rattus norvegicus | | NP_037290.3 | | |
| 29 | Rattus norvegicus | forward | | GI: 1937883835 | NM_013158.3 |
| 30 | Rattus norvegicus | reverse | | GI: 1937883835 | NM_013158.3 |
| | | complement | | | |

TABLE 2
Abbreviations of nucleotide monomers used in nucleic
acid sequence representation. It will be understood
that these monomers, when present in an oligonucleotide,
are mutually linked by 5′-3′-phosphodiester bonds.

| Abbreviation | Nucleotide(s) |
|---|---|
| A | Adenosine-3′-phosphate |
| Af | 2′-fluoroadenosine-3′-phosphate |
| Afs | 2′-fluoroadenosine-3′-phosphorothioate |
| As | adenosine-3′-phosphorothioate |
| C | cytidine-3′-phosphate |
| Cf | 2′-fluorocytidine-3′-phosphate |
| Cfs | 2′-fluorocytidine-3′-phosphorothioate |
| Cs | cytidine-3′-phosphorothioate |
| G | guanosine-3′-phosphate |
| Gf | 2′-fluoroguanosine-3′-phosphate |
| Gfs | 2′-fluoroguanosine-3′-phosphorothioate |
| Gs | guanosine-3′-phosphorothioate |
| T | 5′-methyluridine-3′-phosphate |
| Tf | 2′-fluoro-5-methyluridine-3′-phosphate |
| Tfs | 2′-fluoro-5-methyluridine-3′-phosphorothioate |
| Ts | 5-methyluridine-3′-phosphorothioate |
| U | Uridine-3′-phosphate |
| Uf | 2′-fluorouridine-3′-phosphate |
| Ufs | 2′-fluorouridine-3′-phosphorothioate |
| Us | uridine-3′-phosphorothioate |
| a | 2′-O-methyladenosine-3′-phosphate |
| as | 2′-O-methyladenosine-3′-phosphorothioate |
| c | 2′-O-methylcytidine-3′-phosphate |
| cs | 2′-O-methylcytidine-3′-phosphorothioate |
| g | 2′-O-methylguanosine-3′-phosphate |
| gs | 2′-O-methylguanosine-3′-phosphorothioate |
| t | 2′-O-methyl-5-methyluridine-3′-phosphate |
| ts | 2′-O-methyl-5-methyluridine-3′-phosphorothioate |
| u | 2′-O-methyluridine-3′-phosphate |
| us | 2′-O-methyluridine-3′-phosphorothioate |
| s | phosphorothioate linkage |
| P | Phosphate |
| dT | 2′-deoxythymidine-3′-phosphate |
| dTs | 2′-deoxythymidine-3′-phosphorothioate |

TABLE 3
Un-modified sense and antisense strand sequences of human DBH siNA agents.

| Duplex | SEQ | Sense sequence | Range in | SEQ | Antisense sequence | Range in |
|---|---|---|---|---|---|---|
| name | ID No. | 5′ to 3′ | NM_000787.4 | ID No. | 5′ to 3′ | NM_000787.4 |
| HS-57-148 | 31 | CGACCGUGGCGAGCUUGAGAA | 279-299 | 55 | UUCUCAAGCUCGCCACGGUCG | 299-279 |
| HS-57-233 | 32 | CACGUACUGGUGCUACAUUAA | 693-713 | 56 | UUAAUGUAGCACCAGUACGUG | 713-693 |
| HS-57-185 | 33 | CCAGGAGAUCCGCAUGUUGAA | 1341-1361 | 57 | UUCAACAUGCGGAUCUCCUGG | 1361-1341 |
| HS-57-182 | 34 | GGAGAUCCGCAUGUUGAAGAA | 1344-1364 | 58 | UUCUUCAACAUGCGGAUCUCC | 1364-1344 |
| HS-57-290 | 35 | UCCUGCACGUACAACACAGAA | 1402-1422 | 59 | UUCUGUGUUGUACGUGCAGGA | 1422-1402 |
| HS-57-260 | 36 | CCUCAUCAACAGGUUCAACAA | 1560-1580 | 60 | UUGUUGAACCUGUUGAUGAGG | 1580-1560 |
| HS-57-141 | 37 | CCGCUUCCAGGGUGAAUGGAA | 1722-1742 | 61 | UUCCAUUCACCCUGGAAGCGG | 1742-1722 |
| HS-57-186 | 38 | AAGGUCAUCUCCACACUGGAA | 1759-1779 | 62 | UUCCAGUGUGGAGAUGACCUU | 1779-1759 |
| HS-62-254 | 39 | CGUGGCGAGCUUGAGAA | 283-299 | 55 | UUCUCAAGCUCGCCACGGUCG | 299-279 |
| HS-62-242 | 40 | UACUGGUGCUACAUUAA | 697-713 | 56 | UUAAUGUAGCACCAGUACGUG | 713-693 |
| HS-62-261 | 41 | GAGAUCCGCAUGUUGAA | 1345-1361 | 57 | UUCAACAUGCGGAUCUCCUGG | 1361-1341 |
| HS-62-234 | 42 | AUCCGCAUGUUGAAGAA | 1348-1364 | 58 | UUCUUCAACAUGCGGAUCUCC | 1364-1344 |
| HS-62-298 | 43 | GCACGUACAACACAGAA | 1406-1422 | 59 | UUCUGUGUUGUACGUGCAGGA | 1422-1402 |
| HS-62-282 | 44 | AUCAACAGGUUCAACAA | 1564-1580 | 60 | UUGUUGAACCUGUUGAUGAGG | 1580-1560 |
| HS-62-205 | 45 | UUCCAGGGUGAAUGGAA | 1726-1742 | 61 | UUCCAUUCACCCUGGAAGCGG | 1742-1722 |
| HS-62-249 | 46 | UCAUCUCCACACUGGAA | 1763-1779 | 62 | UUCCAGUGUGGAGAUGACCUU | 1779-1759 |
| HS-31-132 | 47 | UGGCGAGCUUGAGAA | 285-299 | 55 | UUCUCAAGCUCGCCACGGUCG | 299-279 |
| HS-31-137 | 48 | CUGGUGCUACAUUAA | 699-713 | 56 | UUAAUGUAGCACCAGUACGUG | 713-693 |
| HS-31-215 | 49 | GAUCCGCAUGUUGAA | 1347-1361 | 57 | UUCAACAUGCGGAUCUCCUGG | 1361-1341 |
| HS-31-189 | 50 | CCGCAUGUUGAAGAA | 1350-1364 | 58 | UUCUUCAACAUGCGGAUCUCC | 1364-1344 |
| HS-31-191 | 51 | ACGUACAACACAGAA | 1408-1422 | 59 | UUCUGUGUUGUACGUGCAGGA | 1422-1402 |
| HS-31-188 | 52 | CAACAGGUUCAACAA | 1566-1580 | 60 | UUGUUGAACCUGUUGAUGAGG | 1580-1560 |
| HS-31-248 | 53 | CCAGGGUGAAUGGAA | 1728-1742 | 61 | UUCCAUUCACCCUGGAAGCGG | 1742-1722 |
| HS-31-127 | 54 | AUCUCCACACUGGAA | 1765-1779 | 62 | UUCCAGUGUGGAGAUGACCUU | 1779-1759 |

TABLE 4
Modified sense and antisense strand sequences of human DBH siNA agents. These sense and antisense sequences are unbranched sequences
containing less than 10 specifically defined nucleotides.

| Duplex | SEQ ID | | SEQ ID | | SEQ ID | |
|---|---|---|---|---|---|---|
| name | No. | Sense sequence 5′ to 3′ | No. | Antisense sequence 5′ to 3′ | No. | mRNA target sequence |
| HS-55-175 | 63 | CfsgsAfcCfgUfgGfcGfaGfcUfuGfaGfsasAf | 87 | usUfscUfcAfaGfcUfcGfcCfsasCfsgsGfsuscsGf | 111 | CGACCGUGGCGAGCUUGAGAA |
| HS-55-176 | 64 | CfsasCfgUfaCfuGfgUfgCfuAfcAfuUfsasAf | 88 | usUfsaAfuGfuAfgCfaCfcAfsgsUfsasCfsgsusGf | 112 | CACGUACUGGUGCUACAUUAA |
| HS-55-187 | 65 | CfscsAfgGfaGfaUfcCfgCfaUfgUfuGfsasAf | 89 | usUfscAfaCfaUfgCfgGfaUfscsUfscsCfsusgsGf | 113 | CCAGGAGAUCCGCAUGUUGAA |
| HS-55-196 | 66 | GfsgsAfgAfuCfcGfcAfuGfuUfgAfaGfsasAf | 90 | usUfscUfuCfaAfcAfuGfcGfsgsAfsusCfsuscsCf | 114 | GGAGAUCCGCAUGUUGAAGAA |
| HS-55-238 | 67 | UfscsCfuGfcAfcGfuAfcAfaCfaCfaGfsasAf | 91 | usUfscUfgUfgUfuGfuAfcGfsusGfscsAfsgsgsAf | 115 | UCCUGCACGUACAACACAGAA |
| HS-55-139 | 68 | CfscsUfcAfuCfaAfcAfgGfuUfcAfaCfsasAf | 92 | usUfsgUfuGfaAfcCfuGfuUfsgsAfsusGfsasgsGf | 116 | CCUCAUCAACAGGUUCAACAA |
| HS-55-197 | 69 | CfscsGfcUfuCfcAfgGfgUfgAfaUfgGfsasAf | 93 | usUfscCfaUfuCfaCfcCfuGfsgsAfsasGfscsgsGf | 117 | CCGCUUCCAGGGUGAAUGGAA |
| HS-55-169 | 70 | AfsasGfgUfcAfuCfuCfcAfcAfcUfgGfsasAf | 94 | usUfscCfaGfuGfuGfgAfgAfsusGfsasCfscsusUf | 118 | AAGGUCAUCUCCACACUGGAA |
| HS-55-203 | 63 | CfsgsAfcCfgUfgGfcGfaGfcUfuGfaGfsasAf | 95 | usUfscUfcAfcGfcUfcGfcCfsasCfsgsGfsUfscsg | 111 | CGACCGUGGCGAGCUUGAGAA |
| HS-55-293 | 64 | CfsasCfgUfaCfuGfgUfgCfuAfcAfuUfsasAf | 96 | usUfsaAfuGfuAfgCfaCfcAfsgsUfsasCfsGfsusg | 112 | CACGUACUGGUGCUACAUUAA |
| HS-55-299 | 65 | CfscsAfgGfaGfaUfcCfgCfaUfgUfuGfsasAf | 97 | usUfscAfaCfaUfgCfgGfaUfscsUfscsCfsUfsgsg | 113 | CCAGGAGAUCCGCAUGUUGAA |
| HS-55-140 | 66 | GfsgsAfgAfuCfcGfcAfuGfuUfgAfaGfsasAf | 98 | usUfscUfuCfaAfcAfuGfcGfsgsAfsusCfsUfscsc | 114 | GGAGAUCCGCAUGUUGAAGAA |
| HS-55-198 | 67 | UfscsCfuGfcAfcGfuAfcAfaCfaCfaGfsasAf | 99 | usUfscUfgUfgUfuGfuAfcGfsusGfscsAfsGfsgsa | 115 | UCCUGCACGUACAACACAGAA |
| HS-55-243 | 68 | CfscsUfcAfuCfaAfcAfgGfuUfcAfaCfsasAf | 100 | usUfsgUfuGfaAfcCfuGfuUfsgsAfsusGfsAfsgsg | 116 | CCUCAUCAACAGGUUCAACAA |
| HS-55-211 | 69 | CfscsGfcUfuCfcAfgGfgUfgAfaUfgGfsasAf | 101 | usUfscCfaUfuCfaCfcCfuGfsgsAfsasGfsCfsgsg | 117 | CCGCUUCCAGGGUGAAUGGAA |
| HS-55-184 | 70 | AfsasGfgUfcAfuCfuCfcAfcAfcUfgGfsasAf | 102 | usUfscCfaGfuGfuGfgAfgAfsusGfsasCfsCfsusu | 118 | AAGGUCAUCUCCACACUGGAA |
| HS-55-263 | 63 | CfsgsAfcCfgUfgGfcGfaGfcUfuGfaGfsasAf | 103 | usUfscUfcAfaGfcUfcGfcCfsasCfsgsGfsusCfsg | 111 | CGACCGUGGCGAGCUUGAGAA |
| HS-55-158 | 64 | CfsasCfgUfaCfuGfgUfgCfuAfcAfuUfsasAf | 104 | usUfsaAfuGfuAfgCfaCfcAfsgsUfsasCfsgsUfsg | 112 | CACGUACUGGUGCUACAUUAA |
| HS-55-219 | 65 | CfscsAfgGfaGfaUfcCfgCfaUfgUfuGfsasAf | 105 | usUfscAfaCfaUfgCfgGfaUfscsUfscsCfsusGfsg | 113 | CCAGGAGAUCCGCAUGUUGAA |
| HS-55-209 | 66 | GfsgsAfgAfuCfcGfcAfuGfuUfgAfaGfsasAf | 106 | usUfscUfuCfaAfcAfuGfcGfsgsAfsusCfsusCfsc | 114 | GGAGAUCCGCAUGUUGAAGAA |
| HS-55-212 | 67 | UfscsCfuGfcAfcGfuAfcAfaCfaCfaGfsasAf | 107 | usUfscUfgUfgUfuGfuAfcGfsusGfscsAfsgsGfsa | 115 | UCCUGCACGUACAACACAGAA |
| HS-55-255 | 68 | CfscsUfcAfuCfaAfcAfgGfuUfcAfaCfsasAf | 108 | usUfsgUfuGfaAfcCfuGfuUfsgsAfsusGfsasGfsg | 116 | CCUCAUCAACAGGUUCAACAA |
| HS-55-294 | 69 | CfscsGfcUfuCfcAfgGfgUfgAfaUfgGfsasAf | 109 | usUfscCfaUfuCfaCfcCfuGfsgsAfsasGfscsGfsg | 117 | CCGCUUCCAGGGUGAAUGGAA |
| HS-55-124 | 70 | AfsasGfgUfcAfuCfuCfcAfcAfcUfgGfsasAf | 110 | usUfscCfaGfuGfuGfgAfgAfsusGfsasCfscsUfsu | 118 | AAGGUCAUCUCCACACUGGAA |
| HS-84-128 | 71 | CfsgsUfgGfcGfaGfcUfuGfaGfsasAf | 87 | usUfscUfcAfaGfcUfcGfcCfsasCfsgsGfsuscsGf | 111 | CGACCGUGGCGAGCUUGAGAA |
| HS-84-194 | 72 | UfsasCfuGfgUfgCfuAfcAfuUfsasAf | 88 | usUfsaAfuGfuAfgCfaCfcAfsgsUfsasCfsgsusGf | 112 | CACGUACUGGUGCUACAUUAA |
| HS-84-226 | 73 | GfsasGfaUfcCfgCfaUfgUfuGfsasAf | 89 | usUfscAfaCfaUfgCfgGfaUfscsUfscsCfsusgsGf | 113 | CCAGGAGAUCCGCAUGUUGAA |
| HS-84-284 | 74 | AfsusCfcGfcAfuGfuUfgAfaGfsasAf | 90 | usUfscUfuCfaAfcAfuGfcGfsgsAfsusCfsuscsCf | 114 | GGAGAUCCGCAUGUUGAAGAA |
| HS-84-172 | 75 | GfscsAfcGfuAfcAfaCfaCfaGfsasAf | 91 | usUfscUfgUfgUfuGfuAfcGfsusGfscsAfsgsgsAf | 115 | UCCUGCACGUACAACACAGAA |
| HS-84-202 | 76 | AfsusCfaAfcAfgGfuUfcAfaCfsasAf | 92 | usUfsgUfuGfaAfcCfuGfuUfsgsAfsusGfsasgsGf | 116 | CCUCAUCAACAGGUUCAACAA |
| HS-84-133 | 77 | UfsusCfcAfgGfgUfgAfaUfgGfsasAf | 93 | usUfscCfaUfuCfaCfcCfuGfsgsAfsasGfscsgsGf | 117 | CCGCUUCCAGGGUGAAUGGAA |
| HS-84-159 | 78 | UfscsAfuCfuCfcAfcAfcUfgGfsasAf | 94 | usUfscCfaGfuGfuGfgAfgAfsusGfsasCfscsusUf | 118 | AAGGUCAUCUCCACACUGGAA |
| HS-84-217 | 71 | CfsgsUfgGfcGfaGfcUfuGfaGfsasAf | 95 | usUfscUfcAfcGfcUfcGfcCfsasCfsgsGfsUfscsg | 111 | CGACCGUGGCGAGCUUGAGAA |
| HS-84-291 | 72 | UfsasCfuGfgUfgCfuAfcAfuUfsasAf | 96 | usUfsaAfuGfuAfgCfaCfcAfsgsUfsasCfsGfsusg | 112 | CACGUACUGGUGCUACAUUAA |
| HS-84-201 | 73 | GfsasGfaUfcCfgCfaUfgUfuGfsasAf | 97 | usUfscAfaCfaUfgCfgGfaUfscsUfscsCfsUfsgsg | 113 | CCAGGAGAUCCGCAUGUUGAA |
| HS-84-283 | 74 | AfsusCfcGfcAfuGfuUfgAfaGfsasAf | 98 | usUfscUfuCfaAfcAfuGfcGfsgsAfsusCfsUfscsc | 114 | GGAGAUCCGCAUGUUGAAGAA |
| HS-84-146 | 75 | GfscsAfcGfuAfcAfaCfaCfaGfsasAf | 99 | usUfscUfgUfgUfuGfuAfcGfsusGfscsAfsGfsgsa | 115 | UCCUGCACGUACAACACAGAA |
| HS-84-229 | 76 | AfsusCfaAfcAfgGfuUfcAfaCfsasAf | 100 | usUfsgUfuGfaAfcCfuGfuUfsgsAfsusGfsAfsgsg | 116 | CCUCAUCAACAGGUUCAACAA |
| HS-84-129 | 77 | UfsusCfcAfgGfgUfgAfaUfgGfsasAf | 101 | usUfscCfaUfuCfaCfcCfuGfsgsAfsasGfsCfsgsg | 117 | CCGCUUCCAGGGUGAAUGGAA |
| HS-84-269 | 78 | UfscsAfuCfuCfcAfcAfcUfgGfsasAf | 102 | usUfscCfaGfuGfuGfgAfgAfsusGfsasCfsCfsusu | 118 | AAGGUCAUCUCCACACUGGAA |
| HS-84-143 | 71 | CfsgsUfgGfcGfaGfcUfuGfaGfsasAf | 103 | usUfscUfcAfaGfcUfcGfcCfsasCfsgsGfsusCfsg | 111 | CGACCGUGGCGAGCUUGAGAA |
| HS-84-131 | 72 | UfsasCfuGfgUfgCfuAfcAfuUfsasAf | 104 | usUfsaAfuGfuAfgCfaCfcAfsgsUfsasCfsgsUfsg | 112 | CACGUACUGGUGCUACAUUAA |
| HS-84-183 | 73 | GfsasGfaUfcCfgCfaUfgUfuGfsasAf | 105 | usUfscAfaCfaUfgCfgGfaUfscsUfscsCfsusGfsg | 113 | CCAGGAGAUCCGCAUGUUGAA |
| HS-84-149 | 74 | AfsusCfcGfcAfuGfuUfgAfaGfsasAf | 106 | usUfscUfuCfaAfcAfuGfcGfsgsAfsusCfsusCfsc | 114 | GGAGAUCCGCAUGUUGAAGAA |
| HS-84-170 | 75 | GfscsAfcGfuAfcAfaCfaCfaGfsasAf | 107 | usUfscUfgUfgUfuGfuAfcGfsusGfscsAfsgsGfsa | 115 | UCCUGCACGUACAACACAGAA |
| HS-84-281 | 76 | AfsusCfaAfcAfgGfuUfcAfaCfsasAf | 108 | usUfsgUfuGfaAfcCfuGfuUfsgsAfsusGfsasGfsg | 116 | CCUCAUCAACAGGUUCAACAA |
| HS-84-228 | 77 | UfsusCfcAfgGfgUfgAfaUfgGfsasAf | 109 | usUfscCfaUfuCfaCfcCfuGfsgsAfsasGfscsGfsg | 117 | CCGCUUCCAGGGUGAAUGGAA |
| HS-84-125 | 78 | UfscsAfuCfuCfcAfcAfcUfgGfsasAf | 110 | usUfscCfaGfuGfuGfgAfgAfsusGfsasCfscsUfsu | 118 | AAGGUCAUCUCCACACUGGAA |
| HS-92-166 | 79 | UfsgsGfcGfaGfcUfuGfaGfsasAf | 87 | usUfscUfcAfaGfcUfcGfcCfsasCfsgsGfsuscsGf | 111 | CGACCGUGGCGAGCUUGAGAA |
| HS-92-259 | 80 | CfsusGfgUfgCfuAfcAfuUfsasAf | 88 | usUfsaAfuGfuAfgCfaCfcAfsgsUfsasCfsgsusGf | 112 | CACGUACUGGUGCUACAUUAA |
| HS-92-195 | 81 | GfsasUfcCfgCfaUfgUfuGfsasAf | 89 | usUfscAfaCfaUfgCfgGfaUfscsUfscsCfsusgsGf | 113 | CCAGGAGAUCCGCAUGUUGAA |
| HS-92-130 | 82 | CfscsGfcAfuGfuUfgAfasGfsaAf | 90 | usUfscUfuCfaAfcAfuGfcGfsgsAfsusCfsuscsCf | 114 | GGAGAUCCGCAUGUUGAAGAA |
| HS-92-267 | 83 | AfscsGfuAfcAfaCfaCfaGfsasAf | 91 | usUfscUfgUfgUfuGfuAfcGfsusGfscsAfsgsgsAf | 115 | UCCUGCACGUACAACACAGAA |
| HS-92-232 | 84 | CfsasAfcAfgGfuUfcAfaCfsasAf | 92 | usUfsgUfuGfaAfcCfuGfuUfsgsAfsusGfsasgsGf | 116 | CCUCAUCAACAGGUUCAACAA |
| HS-92-216 | 85 | CfscsAfgGfgUfgAfaUfgGfsasAf | 93 | usUfscCfaUfuCfaCfcCfuGfsgsAfsasGfscsgsGf | 117 | CCGCUUCCAGGGUGAAUGGAA |
| HS-92-296 | 86 | AfsusCfuCfcAfcAfcUfgGfsasAf | 94 | usUfscCfaGfuGfuGfgAfgAfsusGfsasCfscsusUf | 118 | AAGGUCAUCUCCACACUGGAA |
| HS-92-181 | 79 | UfsgsGfcGfaGfcUfuGfaGfsasAf | 95 | usUfscUfcAfcGfcUfcGfcCfsasCfsgsGfsUfscsg | 111 | CGACCGUGGCGAGCUUGAGAA |
| HS-92-220 | 80 | CfsusGfgUfgCfuAfcAfuUfsasAf | 96 | usUfsaAfuGfuAfgCfaCfcAfsgsUfsasCfsGfsusg | 112 | CACGUACUGGUGCUACAUUAA |
| HS-92-286 | 81 | GfsasUfcCfgCfaUfgUfuGfsasAf | 97 | usUfscAfaCfaUfgCfgGfaUfscsUfscsCfsUfsgsg | 113 | CCAGGAGAUCCGCAUGUUGAA |
| HS-92-155 | 82 | CfscsGfcAfuGfuUfgAfasGfsaAf | 98 | usUfscUfuCfaAfcAfuGfcGfsgsAfsusCfsUfscsc | 114 | GGAGAUCCGCAUGUUGAAGAA |
| HS-92-174 | 83 | AfscsGfuAfcAfaCfaCfaGfsasAf | 99 | usUfscUfgUfgUfuGfuAfcGfsusGfscsAfsGfsgsa | 115 | UCCUGCACGUACAACACAGAA |
| HS-92-236 | 84 | CfsasAfcAfgGfuUfcAfaCfsasAf | 100 | usUfsgUfuGfaAfcCfuGfuUfsgsAfsusGfsAfsgsg | 116 | CCUCAUCAACAGGUUCAACAA |
| HS-92-221 | 85 | CfscsAfgGfgUfgAfaUfgGfsasAf | 101 | usUfscCfaUfuCfaCfcCfuGfsgsAfsasGfsCfsgsg | 117 | CCGCUUCCAGGGUGAAUGGAA |
| HS-92-153 | 86 | AfsusCfuCfcAfcAfcUfgGfsasAf | 102 | usUfscCfaGfuGfuGfgAfgAfsusGfsasCfsCfsusu | 118 | AAGGUCAUCUCCACACUGGAA |
| HS-92-285 | 79 | UfsgsGfcGfaGfcUfuGfaGfsasAf | 103 | usUfscUfcAfaGfcUfcGfcCfsasCfsgsGfsusCfsg | 111 | CGACCGUGGCGAGCUUGAGAA |
| HS-92-288 | 80 | CfsusGfgUfgCfuAfcAfuUfsasAf | 104 | usUfsaAfuGfuAfgCfaCfcAfsgsUfsasCfsgsUfsg | 112 | CACGUACUGGUGCUACAUUAA |
| HS-92-245 | 81 | GfsasUfcCfgCfaUfgUfuGfsasAf | 105 | usUfscAfaCfaUfgCfgGfaUfscsUfscsCfsusGfsg | 113 | CCAGGAGAUCCGCAUGUUGAA |
| HS-92-134 | 82 | CfscsGfcAfuGfuUfgAfasGfsaAf | 106 | usUfscUfuCfaAfcAfuGfcGfsgsAfsusCfsusCfsc | 114 | GGAGAUCCGCAUGUUGAAGAA |
| HS-92-225 | 83 | AfscsGfuAfcAfaCfaCfaGfsasAf | 107 | usUfscUfgUfgUfuGfuAfcGfsusGfscsAfsgsGfsa | 115 | UCCUGCACGUACAACACAGAA |
| HS-92-157 | 84 | CfsasAfcAfgGfuUfcAfaCfsasAf | 108 | usUfsgUfuGfaAfcCfuGfuUfsgsAfsusGfsasGfsg | 116 | CCUCAUCAACAGGUUCAACAA |
| HS-92-171 | 85 | CfscsAfgGfgUfgAfaUfgGfsasAf | 109 | usUfscCfaUfuCfaCfcCfuGfsgsAfsasGfscsGfsg | 117 | CCGCUUCCAGGGUGAAUGGAA |
| HS-92-180 | 86 | AfsusCfuCfcAfcAfcUfgGfsasAf | 110 | usUfscCfaGfuGfuGfgAfgAfsusGfsasCfscsUfsu | 118 | AAGGUCAUCUCCACACUGGAA |

TABLE 5
Modified sense and antisense strand sequences of human DBH siNA agents. These sense and antisense sequences are unbranched sequences
containing less than 10 specifically defined nucleotides.

| Duplex | SEQ ID | | SEQ ID | | SEQ ID | |
|---|---|---|---|---|---|---|
| name | No. | Sense sequence 5′ to 3′ | No. | Antisense sequence 5′ to 3′ | No. | mRNA target sequence |
| HS-81-239 | 119 | CfgAfcCfgUfgGfcGfaGfcUfuGfaGfaAfsdTdT | 143 | usUfscUfcAfaGfcUfcGfcCfaCfgGfuCfgsdTdT | 111 | CGACCGUGGCGAGCUUGAGAA |
| HS-81-264 | 120 | CfaCfgUfaCfuGfgUfgCfuAfcAfuUfaAfsdTdT | 144 | usUfsaAfuGfuAfgCfaCfcAfgUfaCfgUfgsdTdT | 112 | CACGUACUGGUGCUACAUUAA |
| HS-81-253 | 121 | CfcAfgGfaGfaUfcCfgCfaUfgUfuGfaAfsdTdT | 145 | usUfscAfaCfaUfgCfgGfaUfcUfcCfuGfgsdTdT | 113 | CCAGGAGAUCCGCAUGUUGAA |
| HS-81-277 | 122 | GfgAfgAfuCfcGfcAfuGfuUfgAfaGfaAfsdTdT | 146 | usUfscUfuCfaAfcAfuGfcGfgAfuCfuCfcsdTdT | 114 | GGAGAUCCGCAUGUUGAAGAA |
| HS-81-244 | 123 | UfcCfuGfcAfcGfuAfcAfaCfaCfaGfaAfsdTdT | 147 | usUfscUfgUfgUfuGfuAfcGfuGfcAfgGfasdTdT | 115 | UCCUGCACGUACAACACAGAA |
| HS-81-241 | 124 | CfcUfcAfuCfaAfcAfgGfuUfcAfaCfaAfsdTdT | 148 | usUfsgUfuGfaAfcCfuGfuUfgAfuGfaGfgsdTdT | 116 | CCUCAUCAACAGGUUCAACAA |
| HS-81-247 | 125 | CfcGfcUfuCfcAfgGfgUfgAfaUfgGfaAfsdTdT | 149 | usUfscCfaUfuCfaCfcCfuGfgAfaGfcGfgsdTdT | 117 | CCGCUUCCAGGGUGAAUGGAA |
| HS-81-126 | 126 | AfaGfgUfcAfuCfuCfcAfcAfcUfgGfaAfsdTdT | 150 | usUfscCfaGfuGfuGfgAfgAfuGfaCfcUfusdTdT | 118 | AAGGUCAUCUCCACACUGGAA |
| HS-85-272 | 127 | CfgAfcCfgUfgGfcGfaGfcUfuGfaGfaAfsusu | 151 | usUfscUfcAfaGfcUfcGfcCfaCfgGfuCfgsusu | 111 | CGACCGUGGCGAGCUUGAGAA |
| HS-85-262 | 128 | CfaCfgUfaCfuGfgUfgCfuAfcAfuUfaAfsusu | 152 | usUfsaAfuGfuAfgCfaCfcAfgUfaCfgUfgsusu | 112 | CACGUACUGGUGCUACAUUAA |
| HS-85-173 | 129 | CfcAfgGfaGfaUfcCfgCfaUfgUfuGfaAfsusu | 153 | usUfscAfaCfaUfgCfgGfaUfcUfcCfuGfgsusu | 113 | CCAGGAGAUCCGCAUGUUGAA |
| HS-85-163 | 130 | GfgAfgAfuCfcGfcAfuGfuUfgAfaGfaAfsusu | 154 | usUfscUfuCfaAfcAfuGfcGfgAfuCfuCfcsusu | 114 | GGAGAUCCGCAUGUUGAAGAA |
| HS-85-231 | 131 | UfcCfuGfcAfcGfuAfcAfaCfaCfaGfaAfsusu | 155 | usUfscUfgUfgUfuGfuAfcGfuGfcAfgGfasusu | 115 | UCCUGCACGUACAACACAGAA |
| HS-85-206 | 132 | CfcUfcAfuCfaAfcAfgGfuUfcAfaCfaAfsusu | 156 | usUfsgUfuGfaAfcCfuGfuUfgAfuGfaGfgsusu | 116 | CCUCAUCAACAGGUUCAACAA |
| HS-85-300 | 133 | CfcGfcUfuCfcAfgGfgUfgAfaUfgGfaAfsusu | 157 | usUfscCfa UfuCfaCfcCfuGfgAfaGfcGfgsusu | 117 | CCGCUUCCAGGGUGAAUGGAA |
| HS-85-227 | 134 | AfaGfgUfcAfuCfuCfcAfcAfcUfgGfaAfsusu | 158 | usUfscCfaGfuGfuGfgAfgAfuGfaCfcUfususu | 118 | AAGGUCAUCUCCACACUGGAA |
| HS-87-165 | 135 | csgsacCfgUfGfGfcgagcuugagaasusu | 159 | usUfscucAfagcucgcCfaCfggucgsusu | 111 | CGACCGUGGCGAGCUUGAGAA |
| HS-87-270 | 136 | csascgUfaCfUfGfgugcuacauuaasusu | 160 | usUfsaauGfuagcaccAfgUfacgugsusu | 112 | CACGUACUGGUGCUACAUUAA |
| HS-87-193 | 137 | cscsagGfaGfAfUfccgcauguugaasusu | 161 | usUfscaaCfaugcggaUfcUfccuggsusu | 113 | CCAGGAGAUCCGCAUGUUGAA |
| HS-87-156 | 138 | gsgsagAfuCfCfGfcauguugaagaasusu | 162 | usUfscuuCfaacaugcGfgAfucuccsusu | 114 | GGAGAUCCGCAUGUUGAAGAA |
| HS-87-230 | 139 | uscscuGfcAfCfGfuacaacacagaasusu | 163 | usUfscugUfguuguacGfuGfcaggasusu | 115 | UCCUGCACGUACAACACAGAA |
| HS-87-279 | 140 | cscsucAfuCfAfAfcagguucaacaasusu | 164 | usUfsguuGfaaccuguUfgAfugaggsusu | 116 | CCUCAUCAACAGGUUCAACAA |
| HS-87-235 | 141 | cscsgcUfuCfCfAfgggugaauggaasusu | 165 | usUfsccaUfucacccuGfgAfagcggsusu | 117 | CCGCUUCCAGGGUGAAUGGAA |
| HS-87-256 | 142 | asasggUfcAfUfCfuccacacuggaasusu | 166 | usUfsccaGfuguggagAfuGfaccuususu | 118 | AAGGUCAUCUCCACACUGGAA |

TABLE 6
Duplexes of interest of human DBH siNA agents.

| | Range in | Sense - | Antisense - |
|---|---|---|---|
| Duplex name | NM_000787.4 | SEQ ID No. | SEQ ID No. |
| Hs-92-166 | 279-299 | 79 | 87 |
| Hs-92-259 | 693-713 | 80 | 88 |
| Hs-92-195 | 1341-1361 | 81 | 89 |
| Hs-92-130 | 1344-1364 | 82 | 90 |
| Hs-92-267 | 1402-1422 | 83 | 91 |
| Hs-92-232 | 1560-1580 | 84 | 92 |
| Hs-92-216 | 1722-1742 | 85 | 93 |
| Hs-92-296 | 1759-1779 | 86 | 94 |
| Hs-92-181 | 279-299 | 79 | 95 |
| Hs-92-220 | 693-713 | 80 | 96 |
| Hs-92-286 | 1341-1361 | 81 | 97 |
| Hs-92-155 | 1344-1364 | 82 | 98 |
| Hs-92-174 | 1402-1422 | 83 | 99 |
| Hs-92-236 | 1560-1580 | 84 | 100 |
| Hs-92-221 | 1722-1742 | 85 | 101 |
| Hs-92-153 | 1759-1779 | 86 | 102 |
| Hs-92-285 | 279-299 | 79 | 103 |
| Hs-92-288 | 693-713 | 80 | 104 |
| Hs-92-245 | 1341-1361 | 81 | 105 |
| Hs-92-134 | 1344-1364 | 82 | 106 |
| Hs-92-225 | 1402-1422 | 83 | 107 |
| Hs-92-157 | 1560-1580 | 84 | 108 |
| Hs-92-171 | 1722-1742 | 85 | 109 |
| Hs-92-180 | 1759-1779 | 86 | 110 |
| Hs-81-239 | 279-299 | 119 | 143 |
| Hs-81-264 | 693-713 | 120 | 144 |
| Hs-81-253 | 1341-1361 | 121 | 145 |
| Hs-81-277 | 1344-1364 | 122 | 146 |
| Hs-81-244 | 1402-1422 | 123 | 147 |
| Hs-81-241 | 1560-1580 | 124 | 148 |
| Hs-81-247 | 1722-1742 | 125 | 149 |
| Hs-81-126 | 1759-1779 | 126 | 150 |
| Hs-85-272 | 279-299 | 127 | 151 |
| Hs-85-262 | 693-713 | 128 | 152 |
| Hs-85-173 | 1341-1361 | 129 | 153 |
| Hs-85-163 | 1344-1364 | 130 | 154 |
| Hs-85-231 | 1402-1422 | 131 | 155 |
| Hs-85-206 | 1560-1580 | 132 | 156 |
| Hs-85-300 | 1722-1742 | 133 | 157 |
| Hs-85-227 | 1759-1779 | 134 | 158 |
| Hs-87-165 | 279-299 | 135 | 159 |
| Hs-87-270 | 693-713 | 136 | 160 |
| Hs-87-193 | 1341-1361 | 137 | 161 |
| Hs-87-156 | 1344-1364 | 138 | 162 |
| Hs-87-230 | 1402-1422 | 139 | 163 |
| Hs-87-279 | 1560-1580 | 140 | 164 |
| Hs-87-235 | 1722-1742 | 141 | 165 |
| Hs-87-256 | 1759-1779 | 142 | 166 |

TABLE 7
Duplexes of major interest of human DBH siNA agents.

| | Range in | Sense - | Antisense - |
|---|---|---|---|
| Duplex name | NM_000787.4 | SEQ ID No. | SEQ ID No. |
| HS-92-195 | 1341-1361 | 81 | 89 |
| HS-92-130 | 1344-1364 | 82 | 90 |
| HS-92-267 | 1402-1422 | 83 | 91 |
| HS-92-216 | 1722-1742 | 85 | 93 |
| HS-92-286 | 1341-1361 | 81 | 97 |
| HS-92-155 | 1344-1364 | 82 | 98 |
| HS-92-174 | 1402-1422 | 83 | 99 |
| HS-92-221 | 1722-1742 | 85 | 101 |
| HS-92-245 | 1341-1361 | 81 | 105 |
| HS-92-134 | 1344-1364 | 82 | 106 |
| HS-92-225 | 1402-1422 | 83 | 107 |
| HS-92-171 | 1722-1742 | 85 | 109 |
| HS-81-253 | 1341-1361 | 121 | 145 |
| HS-81-277 | 1344-1364 | 122 | 146 |
| HS-81-244 | 1402-1422 | 123 | 147 |
| HS-81-247 | 1722-1742 | 125 | 149 |
| HS-85-173 | 1341-1361 | 129 | 153 |
| HS-85-163 | 1344-1364 | 130 | 154 |
| HS-85-231 | 1402-1422 | 131 | 155 |
| HS-85-300 | 1722-1742 | 133 | 157 |
| HS-87-193 | 1341-1361 | 137 | 161 |
| HS-87-156 | 1344-1364 | 138 | 162 |
| HS-87-230 | 1402-1422 | 139 | 163 |
| HS-87-235 | 1722-1742 | 142 | 166 |

TABLE 8
mRNA DBH target sequences in non-human mammalian species.

| Duplex | | Nucleotide GenBank | SEQ ID | | |
|---|---|---|---|---|---|
| name | Species | Accession No | No. | mRNA target sequence | Range in |
| Mm-92-195 | Macaca mulatta | NM_001278345.2 | 113 | CCAGGAGAUCCGCAUGUUGAA | 1341-1361 |
| Mf-92-195 | Macaca fascicularis | XM_005580497.3 | 113 | CCAGGAGAUCCGCAUGUUGAA | 1350-1370 |
| Clf-92-195 | Canis lupus | NM_001005263.1 | 113 | CCAGGAGAUCCGCAUGUUGAA | 1316-1336 |
| | familiaris | | | | |
| Ec-92-195 | Equus caballus | NM_001081770.3 | 113 | CCAGGAGAUCCGCAUGUUGAA | 1316-1336 |
| Bt-92-195 | Bos taurus | NM_180995.2 | 113 | CCAGGAGAUCCGCAUGUUGAA | 1318-1338 |
| Fc-92-195 | Felis catus | XM_003996036.6 | 113 | CCAGGAGAUCCGCAUGUUGAA | 1814-1834 |
| Mu-92-195 | Mus musculus | NM_138942.3 | 223 | CCAGGAGAUCAGAAUGCUGAA | 1353-1373 |
| Rn-92-195 | Rattus norvegicus | NM_013158.3 | 224 | UCAGGAGAUCAGAAUGCUGAA | 1347-1367 |
| Mm-92-130 | Macaca mulatta | NM_001278345.2 | 114 | GGAGAUCCGCAUGUUGAAGAA | 1344-1364 |
| Mf-92-130 | Macaca fascicularis | XM_005580497.3 | 114 | GGAGAUCCGCAUGUUGAAGAA | 1353-1373 |
| Clf-92-130 | Canis lupus | NM_001005263.1 | 114 | GGAGAUCCGCAUGUUGAAGAA | 1319-1339 |
| | familiaris | | | | |
| Ec-92-130 | Equus caballus | NM_001081770.3 | 114 | GGAGAUCCGCAUGUUGAAGAA | 1319-1339 |
| Bt-92-130 | Bos taurus | NM_180995.2 | 114 | GGAGAUCCGCAUGUUGAAGAA | 1321-1341 |
| Fc-92-130 | Felis catus | XM_003996036.6 | 114 | GGAGAUCCGCAUGUUGAAGAA | 1817-1837 |
| Mu-92-130 | Mus musculus | NM_138942.3 | 225 | GGAGAUCAGAAUGCUGAAGAA | 1356-1376 |
| Rn-92-130 | Rattus norvegicus | NM_013158.3 | 225 | GGAGAUCAGAAUGCUGAAGAA | 1350-1370 |
| Mm-92-267 | Macaca mulatta | NM_001278345.2 | 226 | UCCUGCACGUACAACACGGAA | 1402-1422 |
| Mf-92-267 | Macaca fascicularis | XM_005580497.3 | 226 | UCCUGCACGUACAACACGGAA | 1411-1431 |
| Clf-92-267 | Canis lupus | NM_001005263.1 | 227 | UCCUGUACAUACAACACAGAA | 1377-1397 |
| | familiaris | | | | |
| Ec-92-267 | Equus caballus | NM_001081770.3 | 228 | UCCUGCACGUACAACACGGAA | 1377-1397 |
| Bt-92-267 | Bos taurus | NM_180995.2 | 229 | UCUUGCACAUACAACACGGAA | 1379-1399 |
| Fc-92-267 | Felis catus | XM_003996036.6 | 230 | UCCUGUACGUACAACACAGAA | 1875-1895 |
| Mu-92-267 | Mus musculus | NM_138942.3 | 231 | UCAUGCACAUACAACACAGAA | 1414-1434 |
| Rn-92-267 | Rattus norvegicus | NM_013158.3 | 232 | UCGUGCACAUACAACACAGAA | 1408-1428 |
| Mm-92-216 | Macaca mulatta | NM_001278345.2 | 233 | CCGCUUCCCGGGUGAAUGGAA | 1722-1742 |
| Mf-92-216 | Macaca fascicularis | XM_005580497.3 | 233 | CCGCUUCCCGGGUGAAUGGAA | 1731-1751 |
| Clf-92-216 | Canis lupus | NM_001005263.1 | 234 | CCGCUUCCCGGGUAAAUGGGA | 1745-1765 |
| | familiaris | | | | |
| Ec-92-216 | Equus caballus | NM_001081770.3 | 235 | GCGAUUCCAGGGUGAAUGGAA | 1697-1717 |
| Bt-92-216 | Bos taurus | NM_180995.2 | 236 | CCGCUUCCAGGGCGAGUGGAA | 1699-1719 |
| Fc-92-216 | Felis catus | XM_003996036.6 | 117 | CCGCUUCCAGGGUGAAUGGAA | 2195-2215 |
| Mu-92-216 | Mus musculus | NM_138942.3 | 237 | CCGCUUCCCGGGUGAGUGGAA | 1734-1754 |
| Rn-92-216 | Rattus norvegicus | NM_013158.3 | 238 | CCGCUUCCCGGGUAACUGGAA | 1728-1748 |
| Mm-92-286 | Macaca mulatta | NM_001278345.2 | 113 | CCAGGAGAUCCGCAUGUUGAA | 1341-1361 |
| Mf-92-286 | Macaca fascicularis | XM_005580497.3 | 113 | CCAGGAGAUCCGCAUGUUGAA | 1350-1370 |
| Clf-92-286 | Canis lupus | NM_001005263.1 | 113 | CCAGGAGAUCCGCAUGUUGAA | 1316-1336 |
| | familiaris | | | | |
| Ec-92-286 | Equus caballus | NM_001081770.3 | 113 | CCAGGAGAUCCGCAUGUUGAA | 1316-1336 |
| Bt-92-286 | Bos taurus | NM_180995.2 | 113 | CCAGGAGAUCCGCAUGUUGAA | 1318-1338 |
| Fc-92-286 | Felis catus | XM_003996036.6 | 113 | CCAGGAGAUCCGCAUGUUGAA | 1814-1834 |
| Mu-92-286 | Mus musculus | NM_138942.3 | 223 | CCAGGAGAUCAGAAUGCUGAA | 1353-1373 |
| Rn-92-286 | Rattus norvegicus | NM_013158.3 | 224 | UCAGGAGAUCAGAAUGCUGAA | 1347-1367 |
| Mm-92-155 | Macaca mulatta | NM_001278345.2 | 114 | GGAGAUCCGCAUGUUGAAGAA | 1344-1364 |
| Mf-92-155 | Macaca fascicularis | XM_005580497.3 | 114 | GGAGAUCCGCAUGUUGAAGAA | 1353-1373 |
| Clf-92-155 | Canis lupus | NM_001005263.1 | 114 | GGAGAUCCGCAUGUUGAAGAA | 1319-1339 |
| | familiaris | | | | |
| Ec-92-155 | Equus caballus | NM_001081770.3 | 114 | GGAGAUCCGCAUGUUGAAGAA | 1319-1339 |
| Bt-92-155 | Bos taurus | NM_180995.2 | 114 | GGAGAUCCGCAUGUUGAAGAA | 1321-1341 |
| Fc-92-155 | Felis catus | XM_003996036.6 | 114 | GGAGAUCCGCAUGUUGAAGAA | 1817-1837 |
| Mu-92-155 | Mus musculus | NM_138942.3 | 225 | GGAGAUCAGAAUGCUGAAGAA | 1356-1376 |
| Rn-92-155 | Rattus norvegicus | NM_013158.3 | 225 | GGAGAUCAGAAUGCUGAAGAA | 1350-1370 |
| Mm-92-174 | Macaca mulatta | NM_001278345.2 | 226 | UCCUGCACGUACAACACGGAA | 1402-1422 |
| Mf-92-174 | Macaca fascicularis | XM_005580497.3 | 226 | UCCUGCACGUACAACACGGAA | 1411-1431 |
| Clf-92-174 | Canis lupus | NM_001005263.1 | 227 | UCCUGUACAUACAACACAGAA | 1377-1397 |
| | familiaris | | | | |
| Ec-92-174 | Equus caballus | NM_001081770.3 | 228 | UCCUGCACGUACAACACGGAA | 1377-1397 |
| Bt-92-174 | Bos taurus | NM_180995.2 | 229 | UCUUGCACAUACAACACGGAA | 1379-1399 |
| Fc-92-174 | Felis catus | XM_003996036.6 | 230 | UCCUGUACGUACAACACAGAA | 1875-1895 |
| Mu-92-174 | Mus musculus | NM_138942.3 | 231 | UCAUGCACAUACAACACAGAA | 1414-1434 |
| Rn-92-174 | Rattus norvegicus | NM_013158.3 | 232 | UCGUGCACAUACAACACAGAA | 1408-1428 |
| Mm-92-221 | Macaca mulatta | NM_001278345.2 | 233 | CCGCUUCCCGGGUGAAUGGAA | 1722-1742 |
| Mf-92-221 | Macaca fascicularis | XM_005580497.3 | 233 | CCGCUUCCCGGGUGAAUGGAA | 1731-1751 |
| Clf-92-221 | Canis lupus | NM_001005263.1 | 234 | CCGCUUCCCGGGUAAAUGGGA | 1745-1765 |
| | familiaris | | | | |
| Ec-92-221 | Equus caballus | NM_001081770.3 | 235 | GCGAUUCCAGGGUGAAUGGAA | 1697-1717 |
| Bt-92-221 | Bos taurus | NM_180995.2 | 236 | CCGCUUCCAGGGCGAGUGGAA | 1699-1719 |
| Fc-92-221 | Felis catus | XM_003996036.6 | 117 | CCGCUUCCAGGGUGAAUGGAA | 2195-2215 |
| Mu-92-221 | Mus musculus | NM_138942.3 | 237 | CCGCUUCCCGGGUGAGUGGAA | 1734-1754 |
| Rn-92-221 | Rattus norvegicus | NM_013158.3 | 238 | CCGCUUCCCGGGUAACUGGAA | 1728-1748 |
| Mm-92-245 | Macaca mulatta | NM_001278345.2 | 113 | CCAGGAGAUCCGCAUGUUGAA | 1341-1361 |
| Mf-92-245 | Macaca fascicularis | XM_005580497.3 | 113 | CCAGGAGAUCCGCAUGUUGAA | 1350-1370 |
| Clf-92-245 | Canis lupus | NM_001005263.1 | 113 | CCAGGAGAUCCGCAUGUUGAA | 1316-1336 |
| | familiaris | | | | |
| Ec-92-245 | Equus caballus | NM_001081770.3 | 113 | CCAGGAGAUCCGCAUGUUGAA | 1316-1336 |
| Bt-92-245 | Bos taurus | NM_180995.2 | 113 | CCAGGAGAUCCGCAUGUUGAA | 1318-1338 |
| Fc-92-245 | Felis catus | XM_003996036.6 | 113 | CCAGGAGAUCCGCAUGUUGAA | 1814-1834 |
| Mu-92-245 | Mus musculus | NM_138942.3 | 223 | CCAGGAGAUCAGAAUGCUGAA | 1353-1373 |
| Rn-92-245 | Rattus norvegicus | NM_013158.3 | 224 | UCAGGAGAUCAGAAUGCUGAA | 1347-1367 |
| Mm-92-134 | Macaca mulatta | NM_001278345.2 | 114 | GGAGAUCCGCAUGUUGAAGAA | 1344-1364 |
| Mf-92-134 | Macaca fascicularis | XM_005580497.3 | 114 | GGAGAUCCGCAUGUUGAAGAA | 1353-1373 |
| Clf-92-134 | Canis lupus | NM_001005263.1 | 114 | GGAGAUCCGCAUGUUGAAGAA | 1319-1339 |
| | familiaris | | | | |
| Ec-92-134 | Equus caballus | NM_001081770.3 | 114 | GGAGAUCCGCAUGUUGAAGAA | 1319-1339 |
| Bt-92-134 | Bos taurus | NM_180995.2 | 114 | GGAGAUCCGCAUGUUGAAGAA | 1321-1341 |
| Fc-92-134 | Felis catus | XM_003996036.6 | 114 | GGAGAUCCGCAUGUUGAAGAA | 1817-1837 |
| Mu-92-134 | Mus musculus | NM_138942.3 | 225 | GGAGAUCAGAAUGCUGAAGAA | 1356-1376 |
| Rn-92-134 | Rattus norvegicus | NM_013158.3 | 225 | GGAGAUCAGAAUGCUGAAGAA | 1350-1370 |
| Mm-92-225 | Macaca mulatta | NM_001278345.2 | 226 | UCCUGCACGUACAACACGGAA | 1402-1422 |
| Mf-92-225 | Macaca fascicularis | XM_005580497.3 | 226 | UCCUGCACGUACAACACGGAA | 1411-1431 |
| Clf-92-225 | Canis lupus | NM_001005263.1 | 227 | UCCUGUACAUACAACACAGAA | 1377-1397 |
| | familiaris | | | | |
| Ec-92-225 | Equus caballus | NM_001081770.3 | 228 | UCCUGCACGUACAACACGGAA | 1377-1397 |
| Bt-92-225 | Bos taurus | NM_180995.2 | 229 | UCUUGCACAUACAACACGGAA | 1379-1399 |
| Fc-92-225 | Felis catus | XM_003996036.6 | 230 | UCCUGUACGUACAACACAGAA | 1875-1895 |
| Mu-92-225 | Mus musculus | NM_138942.3 | 231 | UCAUGCACAUACAACACAGAA | 1414-1434 |
| Rn-92-225 | Rattus norvegicus | NM_013158.3 | 232 | UCGUGCACAUACAACACAGAA | 1408-1428 |
| Mm-92-171 | Macaca mulatta | NM_001278345.2 | 233 | CCGCUUCCCGGGUGAAUGGAA | 1722-1742 |
| Mf-92-171 | Macaca fascicularis | XM_005580497.3 | 233 | CCGCUUCCCGGGUGAAUGGAA | 1731-1751 |
| Clf-92-171 | Canis lupus | NM_001005263.1 | 234 | CCGCUUCCCGGGUAAAUGGGA | 1745-1765 |
| | familiaris | | | | |
| Ec-92-171 | Equus caballus | NM_001081770.3 | 235 | GCGAUUCCAGGGUGAAUGGAA | 1697-1717 |
| Bt-92-171 | Bos taurus | NM_180995.2 | 236 | CCGCUUCCAGGGCGAGUGGAA | 1699-1719 |
| Fc-92-171 | Felis catus | XM_003996036.6 | 117 | CCGCUUCCAGGGUGAAUGGAA | 2195-2215 |
| Mu-92-171 | Mus musculus | NM_138942.3 | 237 | CCGCUUCCCGGGUGAGUGGAA | 1734-1754 |
| Rn-92-171 | Rattus norvegicus | NM_013158.3 | 238 | CCGCUUCCCGGGUAACUGGAA | 1728-1748 |
| Mm-81-253 | Macaca mulatta | NM_001278345.2 | 113 | CCAGGAGAUCCGCAUGUUGAA | 1341-1361 |
| Mf-81-253 | Macaca fascicularis | XM_005580497.3 | 113 | CCAGGAGAUCCGCAUGUUGAA | 1350-1370 |
| Clf-81-253 | Canis lupus | NM_001005263.1 | 113 | CCAGGAGAUCCGCAUGUUGAA | 1316-1336 |
| | familiaris | | | | |
| Ec-81-253 | Equus caballus | NM_001081770.3 | 113 | CCAGGAGAUCCGCAUGUUGAA | 1316-1336 |
| Bt-81-253 | Bos taurus | NM_180995.2 | 113 | CCAGGAGAUCCGCAUGUUGAA | 1318-1338 |
| Fc-81-253 | Felis catus | XM_003996036.6 | 113 | CCAGGAGAUCCGCAUGUUGAA | 1814-1834 |
| Mu-81-253 | Mus musculus | NM_138942.3 | 223 | CCAGGAGAUCAGAAUGCUGAA | 1353-1373 |
| Rn-81-253 | Rattus norvegicus | NM_013158.3 | 224 | UCAGGAGAUCAGAAUGCUGAA | 1347-1367 |
| Mm-81-277 | Macaca mulatta | NM_001278345.2 | 114 | GGAGAUCCGCAUGUUGAAGAA | 1344-1364 |
| Mf-81-277 | Macaca fascicularis | XM_005580497.3 | 114 | GGAGAUCCGCAUGUUGAAGAA | 1353-1373 |
| Clf-81-277 | Canis lupus | NM_001005263.1 | 114 | GGAGAUCCGCAUGUUGAAGAA | 1319-1339 |
| | familiaris | | | | |
| Ec-81-277 | Equus caballus | NM_001081770.3 | 114 | GGAGAUCCGCAUGUUGAAGAA | 1319-1339 |
| Bt-81-277 | Bos taurus | NM_180995.2 | 114 | GGAGAUCCGCAUGUUGAAGAA | 1321-1341 |
| Fc-81-277 | Felis catus | XM_003996036.6 | 114 | GGAGAUCCGCAUGUUGAAGAA | 1817-1837 |
| Mu-81-277 | Mus musculus | NM_138942.3 | 225 | GGAGAUCAGAAUGCUGAAGAA | 1356-1376 |
| Rn-81-277 | Rattus norvegicus | NM_013158.3 | 225 | GGAGAUCAGAAUGCUGAAGAA | 1350-1370 |
| Mm-81-244 | Macaca mulatta | NM_001278345.2 | 226 | UCCUGCACGUACAACACGGAA | 1402-1422 |
| Mf-81-244 | Macaca fascicularis | XM_005580497.3 | 226 | UCCUGCACGUACAACACGGAA | 1411-1431 |
| Clf-81-244 | Canis lupus | NM_001005263.1 | 227 | UCCUGUACAUACAACACAGAA | 1377-1397 |
| | familiaris | | | | |
| Ec-81-244 | Equus caballus | NM_001081770.3 | 228 | UCCUGCACGUACAACACGGAA | 1377-1397 |
| Bt-81-244 | Bos taurus | NM_180995.2 | 229 | UCUUGCACAUACAACACGGAA | 1379-1399 |
| Fc-81-244 | Felis catus | XM_003996036.6 | 230 | UCCUGUACGUACAACACAGAA | 1875-1895 |
| Mu-81-244 | Mus musculus | NM_138942.3 | 231 | UCAUGCACAUACAACACAGAA | 1414-1434 |
| Rn-81-244 | Rattus norvegicus | NM_013158.3 | 232 | UCGUGCACAUACAACACAGAA | 1408-1428 |
| Mm-81-247 | Macaca mulatta | NM_001278345.2 | 233 | CCGCUUCCCGGGUGAAUGGAA | 1722-1742 |
| Mf-81-247 | Macaca fascicularis | XM_005580497.3 | 233 | CCGCUUCCCGGGUGAAUGGAA | 1731-1751 |
| Clf-81-247 | Canis lupus | NM_001005263.1 | 234 | CCGCUUCCCGGGUAAAUGGGA | 1745-1765 |
| | familiaris | | | | |
| Ec-81-247 | Equus caballus | NM_001081770.3 | 235 | GCGAUUCCAGGGUGAAUGGAA | 1697-1717 |
| Bt-81-247 | Bos taurus | NM_180995.2 | 236 | CCGCUUCCAGGGCGAGUGGAA | 1699-1719 |
| Fc-81-247 | Felis catus | XM_003996036.6 | 117 | CCGCUUCCAGGGUGAAUGGAA | 2195-2215 |
| Mu-81-247 | Mus musculus | NM_138942.3 | 237 | CCGCUUCCCGGGUGAGUGGAA | 1734-1754 |
| Rn-81-247 | Rattus norvegicus | NM_013158.3 | 238 | CCGCUUCCCGGGUAACUGGAA | 1728-1748 |
| Mm-85-173 | Macaca mulatta | NM_001278345.2 | 113 | CCAGGAGAUCCGCAUGUUGAA | 1341-1361 |
| Mf-85-173 | Macaca fascicularis | XM_005580497.3 | 113 | CCAGGAGAUCCGCAUGUUGAA | 1350-1370 |
| Clf-85-173 | Canis lupus | NM_001005263.1 | 113 | CCAGGAGAUCCGCAUGUUGAA | 1316-1336 |
| | familiaris | | | | |
| Ec-85-173 | Equus caballus | NM_001081770.3 | 113 | CCAGGAGAUCCGCAUGUUGAA | 1316-1336 |
| Bt-85-173 | Bos taurus | NM_180995.2 | 113 | CCAGGAGAUCCGCAUGUUGAA | 1318-1338 |
| Fc-85-173 | Felis catus | XM_003996036.6 | 113 | CCAGGAGAUCCGCAUGUUGAA | 1814-1834 |
| Mu-85-173 | Mus musculus | NM_138942.3 | 223 | CCAGGAGAUCAGAAUGCUGAA | 1353-1373 |
| Rn-85-173 | Rattus norvegicus | NM_013158.3 | 224 | UCAGGAGAUCAGAAUGCUGAA | 1347-1367 |
| Mm-85-163 | Macaca mulatta | NM_001278345.2 | 114 | GGAGAUCCGCAUGUUGAAGAA | 1344-1364 |
| Mf-85-163 | Macaca fascicularis | XM_005580497.3 | 114 | GGAGAUCCGCAUGUUGAAGAA | 1353-1373 |
| Clf-85-163 | Canis lupus | NM_001005263.1 | 114 | GGAGAUCCGCAUGUUGAAGAA | 1319-1339 |
| | familiaris | | | | |
| Ec-85-163 | Equus caballus | NM_001081770.3 | 114 | GGAGAUCCGCAUGUUGAAGAA | 1319-1339 |
| Bt-85-163 | Bos taurus | NM_180995.2 | 114 | GGAGAUCCGCAUGUUGAAGAA | 1321-1341 |
| Fc-85-163 | Felis catus | XM_003996036.6 | 114 | GGAGAUCCGCAUGUUGAAGAA | 1817-1837 |
| Mu-85-163 | Mus musculus | NM_138942.3 | 225 | GGAGAUCAGAAUGCUGAAGAA | 1356-1376 |
| Rn-85-163 | Rattus norvegicus | NM_013158.3 | 225 | GGAGAUCAGAAUGCUGAAGAA | 1350-1370 |
| Mm-85-231 | Macaca mulatta | NM_001278345.2 | 226 | UCCUGCACGUACAACACGGAA | 1402-1422 |
| Mf-85-231 | Macaca fascicularis | XM_005580497.3 | 226 | UCCUGCACGUACAACACGGAA | 1411-1431 |
| Clf-85-231 | Canis lupus | NM_001005263.1 | 227 | UCCUGUACAUACAACACAGAA | 1377-1397 |
| | familiaris | | | | |
| Ec-85-231 | Equus caballus | NM_001081770.3 | 228 | UCCUGCACGUACAACACGGAA | 1377-1397 |
| Bt-85-231 | Bos taurus | NM_180995.2 | 229 | UCUUGCACAUACAACACGGAA | 1379-1399 |
| Fc-85-231 | Felis catus | XM_003996036.6 | 230 | UCCUGUACGUACAACACAGAA | 1875-1895 |
| Mu-85-231 | Mus musculus | NM_138942.3 | 231 | UCAUGCACAUACAACACAGAA | 1414-1434 |
| Rn-85-231 | Rattus norvegicus | NM_013158.3 | 232 | UCGUGCACAUACAACACAGAA | 1408-1428 |
| Mm-85-300 | Macaca mulatta | NM_001278345.2 | 233 | CCGCUUCCCGGGUGAAUGGAA | 1722-1742 |
| Mf-85-300 | Macaca fascicularis | XM_005580497.3 | 233 | CCGCUUCCCGGGUGAAUGGAA | 1731-1751 |
| Clf-85-300 | Canis lupus | NM_001005263.1 | 234 | CCGCUUCCCGGGUAAAUGGGA | 1745-1765 |
| | familiaris | | | | |
| Ec-85-300 | Equus caballus | NM_001081770.3 | 235 | GCGAUUCCAGGGUGAAUGGAA | 1697-1717 |
| Bt-85-300 | Bos taurus | NM_180995.2 | 236 | CCGCUUCCAGGGCGAGUGGAA | 1699-1719 |
| Fc-85-300 | Felis catus | XM_003996036.6 | 117 | CCGCUUCCAGGGUGAAUGGAA | 2195-2215 |
| Mu-85-300 | Mus musculus | NM_138942.3 | 237 | CCGCUUCCCGGGUGAGUGGAA | 1734-1754 |
| Rn-85-300 | Rattus norvegicus | NM_013158.3 | 238 | CCGCUUCCCGGGUAACUGGAA | 1728-1748 |
| Mm-87-193 | Macaca mulatta | NM_001278345.2 | 113 | CCAGGAGAUCCGCAUGUUGAA | 1341-1361 |
| Mf-87-193 | Macaca fascicularis | XM_005580497.3 | 113 | CCAGGAGAUCCGCAUGUUGAA | 1350-1370 |
| Clf-87-193 | Canis lupus | NM_001005263.1 | 113 | CCAGGAGAUCCGCAUGUUGAA | 1316-1336 |
| | familiaris | | | | |
| Ec-87-193 | Equus caballus | NM_001081770.3 | 113 | CCAGGAGAUCCGCAUGUUGAA | 1316-1336 |
| Bt-87-193 | Bos taurus | NM_180995.2 | 113 | CCAGGAGAUCCGCAUGUUGAA | 1318-1338 |
| Fc-87-193 | Felis catus | XM_003996036.6 | 113 | CCAGGAGAUCCGCAUGUUGAA | 1814-1834 |
| Mu-87-193 | Mus musculus | NM_138942.3 | 223 | CCAGGAGAUCAGAAUGCUGAA | 1353-1373 |
| Rn-87-193 | Rattus norvegicus | NM_013158.3 | 224 | UCAGGAGAUCAGAAUGCUGAA | 1347-1367 |
| Mm-87-156 | Macaca mulatta | NM_001278345.2 | 114 | GGAGAUCCGCAUGUUGAAGAA | 1344-1364 |
| Mf-87-156 | Macaca fascicularis | XM_005580497.3 | 114 | GGAGAUCCGCAUGUUGAAGAA | 1353-1373 |
| Clf-87-156 | Canis lupus | NM_001005263.1 | 114 | GGAGAUCCGCAUGUUGAAGAA | 1319-1339 |
| | familiaris | | | | |
| Ec-87-156 | Equus caballus | NM_001081770.3 | 114 | GGAGAUCCGCAUGUUGAAGAA | 1319-1339 |
| Bt-87-156 | Bos taurus | NM_180995.2 | 114 | GGAGAUCCGCAUGUUGAAGAA | 1321-1341 |
| Fc-87-156 | Felis catus | XM_003996036.6 | 114 | GGAGAUCCGCAUGUUGAAGAA | 1817-1837 |
| Mu-87-156 | Mus musculus | NM_138942.3 | 225 | GGAGAUCAGAAUGCUGAAGAA | 1356-1376 |
| Rn-87-156 | Rattus norvegicus | NM_013158.3 | 225 | GGAGAUCAGAAUGCUGAAGAA | 1350-1370 |
| Mm-87-230 | Macaca mulatta | NM_001278345.2 | 226 | UCCUGCACGUACAACACGGAA | 1402-1422 |
| Mf-87-230 | Macaca fascicularis | XM_005580497.3 | 226 | UCCUGCACGUACAACACGGAA | 1411-1431 |
| Clf-87-230 | Canis lupus | NM_001005263.1 | 227 | UCCUGUACAUACAACACAGAA | 1377-1397 |
| | familiaris | | | | |
| Ec-87-230 | Equus caballus | NM_001081770.3 | 228 | UCCUGCACGUACAACACGGAA | 1377-1397 |
| Bt-87-230 | Bos taurus | NM_180995.2 | 229 | UCUUGCACAUACAACACGGAA | 1379-1399 |
| Fc-87-230 | Felis catus | XM_003996036.6 | 230 | UCCUGUACGUACAACACAGAA | 1875-1895 |
| Mu-87-230 | Mus musculus | NM_138942.3 | 231 | UCAUGCACAUACAACACAGAA | 1414-1434 |
| Rn-87-230 | Rattus norvegicus | NM_013158.3 | 232 | UCGUGCACAUACAACACAGAA | 1408-1428 |
| Mm-87-235 | Macaca mulatta | NM_001278345.2 | 233 | CCGCUUCCCGGGUGAAUGGAA | 1722-1742 |
| Mf-87-235 | Macaca fascicularis | XM_005580497.3 | 233 | CCGCUUCCCGGGUGAAUGGAA | 1731-1751 |
| Clf-87-235 | Canis lupus | NM_001005263.1 | 234 | CCGCUUCCCGGGUAAAUGGGA | 1745-1765 |
| | familiaris | | | | |
| Ec-87-235 | Equus caballus | NM_001081770.3 | 235 | GCGAUUCCAGGGUGAAUGGAA | 1697-1717 |
| Bt-87-235 | Bos taurus | NM_180995.2 | 236 | CCGCUUCCAGGGCGAGUGGAA | 1699-1719 |
| Fc-87-235 | Felis catus | XM_003996036.6 | 117 | CCGCUUCCAGGGUGAAUGGAA | 2195-2215 |
| Mu-87-235 | Mus musculus | NM_138942.3 | 237 | CCGCUUCCCGGGUGAGUGGAA | 1734-1754 |
| Rn-87-235 | Rattus norvegicus | NM_013158.3 | 238 | CCGCUUCCCGGGUAACUGGAA | 1728-1748 |

TABLE 9
Modified sense and antisense strand sequences of non-human mammalian DBH siNA agents. These sense and antisense sequences are unbranched
sequences containing less than 10 specifically defined nucleotides.

| | SEQ | | SEQ ID | |
|---|---|---|---|---|
| Duplex name | ID No. | Sense sequence 5′ to 3′ | No. | Antisense sequence 5′ to 3′ |
| Mm-92-195 | 81 | GfsasUfcCfgCfaUfgUfuGfsasAf | 89 | usUfscAfaCfaUfgCfgGfaUfscsUfscsCfsusgsGf |
| Mf-92-195 | 81 | GfsasUfcCfgCfaUfgUfuGfsasAf | 89 | usUfscAfaCfaUfgCfgGfaUfscsUfscsCfsusgsGf |
| Clf-92-195 | 81 | GfsasUfcCfgCfaUfgUfuGfsasAf | 89 | usUfscAfaCfaUfgCfgGfaUfscsUfscsCfsusgsGf |
| Ec-92-195 | 81 | GfsasUfcCfgCfaUfgUfuGfsasAf | 89 | usUfscAfaCfaUfgCfgGfaUfscsUfscsCfsusgsGf |
| Bt-92-195 | 81 | GfsasUfcCfgCfaUfgUfuGfsasAf | 89 | usUfscAfaCfaUfgCfgGfaUfscsUfscsCfsusgsGf |
| Fc-92-195 | 81 | GfsasUfcCfgCfaUfgUfuGfsasAf | 89 | usUfscAfaCfaUfgCfgGfaUfscsUfscsCfsusgsGf |
| Mu-92-195 | 167 | GfsasUfcAfgAfaUfgCfuGfsasAf | 176 | usUfscAfgCfaUfuCfuGfaUfscsUfscsCfsusgsGf |
| Rn-92-195 | 167 | GfsasUfcAfgAfaUfgCfuGfsasAf | 177 | usUfscAfgCfaUfuCfuGfaUfscsUfscsCfsusgsAf |
| Mm-92-130 | 82 | CfscsGfcAfuGfuUfgAfasGfsaAf | 90 | usUfscUfuCfaAfcAfuGfcGfsgsAfsusCfsuscsCf |
| Mf-92-130 | 82 | CfscsGfcAfuGfuUfgAfasGfsaAf | 90 | usUfscUfuCfaAfcAfuGfcGfsgsAfsusCfsuscsCf |
| Clf-92-130 | 82 | CfscsGfcAfuGfuUfgAfasGfsaAf | 90 | usUfscUfuCfaAfcAfuGfcGfsgsAfsusCfsuscsCf |
| Ec-92-130 | 82 | CfscsGfcAfuGfuUfgAfasGfsaAf | 90 | usUfscUfuCfaAfcAfuGfcGfsgsAfsusCfsuscsCf |
| Bt-92-130 | 82 | CfscsGfcAfuGfuUfgAfasGfsaAf | 90 | usUfscUfuCfaAfcAfuGfcGfsgsAfsusCfsuscsCf |
| Fc-92-130 | 82 | CfscsGfcAfuGfuUfgAfasGfsaAf | 90 | usUfscUfuCfaAfcAfuGfcGfsgsAfsusCfsuscsCf |
| Mu-92-130 | 168 | CfsasGfaAfuGfcUfgAfaGfsasAf | 178 | usUfscUfuCfaGfcAfuUfcUfsgsAfsusCfsuscsCf |
| Rn-92-130 | 168 | CfsasGfaAfuGfcUfgAfaGfsasAf | 178 | usUfscUfuCfaGfcAfuUfcUfsgsAfsusCfsuscsCf |
| Mm-92-267 | 169 | AfscsGfuAfcAfaCfaCfgGfsasAf | 179 | usUfscCfgUfgUfuGfuAfcGfsusGfscsAfsgsgsAf |
| Mf-92-267 | 169 | AfscsGfuAfcAfaCfaCfgGfsasAf | 179 | usUfscCfgUfgUfuGfuAfcGfsusGfscsAfsgsgsAf |
| Clf-92-267 | 170 | AfscsAfuAfcAfaCfaCfaGfsasAf | 180 | usUfscUfgUfgUfuGfuAfuGfsusAfscsAfsgsgsAf |
| Ec-92-267 | 171 | AfscsGfuAfcAfaCfaCfgGfsasAf | 181 | usUfscCfgUfgUfuGfuAfcGfsusGfscsAfsgsgsAf |
| Bt-92-267 | 172 | AfscsAfuAfcAfaCfaCfgGfsasAf | 182 | usUfscCfgUfgUfuGfuAfuGfsusGfscsAfsasgsAf |
| Fc-92-267 | 83 | AfscsGfuAfcAfaCfaCfaGfsasAf | 183 | usUfscUfgUfgUfuGfuAfcGfsusAfscsAfsgsgsAf |
| Mu-92-267 | 83 | AfscsGfuAfcAfaCfaCfaGfsasAf | 184 | usUfscUfgUfgUfuGfuAfuGfsusGfscsAfsusgsAf |
| Rn-92-267 | 170 | AfscsAfuAfcAfaCfaCfaGfsasAf | 184 | usUfscUfgUfgUfuGfuAfuGfsusGfscsAfscsgsAf |
| Mm-92-216 | 171 | CfscsCfgGfgUfgAfaUfgGfsasAf | 185 | usUfscCfaUfuCfaCfcCfgGfsgsAfsasGfscsgsGf |
| Mf-92-216 | 171 | CfscsCfgGfgUfgAfaUfgGfsasAf | 185 | usUfscCfaUfuCfaCfcCfgGfsgsAfsasGfscsgsGf |
| Clf-92-216 | 172 | CfscsCfgGfgUfaAfaUfgGfsgsAf | 186 | usUfscCfaUfuUfaCfcCfgGfsgsAfsasGfscsgsGf |
| Ec-92-216 | 85 | CfscsAfgGfgUfgAfaUfgGfsasAf | 187 | usUfscCfaUfuCfaCfcCfuGfsgsAfsasUfscsgsCf |
| Bt-92-216 | 173 | CfscsAfgGfgCfgAfgUfgGfsasAf | 188 | usUfscCfaCfuCfgCfcCfuGfsgsAfsasGfscsgsGf |
| Fc-92-216 | 85 | CfscsAfgGfgUfgAfaUfgGfsasAf | 93 | usUfscCfaUfuCfaCfcCfuGfsgsAfsasGfscsgsGf |
| Mu-92-216 | 174 | CfscsCfgGfgUfgAfgUfgGfsasAf | 189 | usUfscCfaCfuCfaCfcCfgGfsgsAfsasGfscsgsGf |
| Rn-92-216 | 175 | CfscsCfgGfgUfaAfcUfgGfsasAf | 190 | usUfscCfaGfuUfaCfcCfgGfsgsAfsasGfscsgsGf |
| Mm-92-286 | 81 | GfsasUfcCfgCfaUfgUfuGfsasAf | 97 | usUfscAfaCfaUfgCfgGfaUfscsUfscsCfsUfsgsg |
| Mf-92-286 | 81 | GfsasUfcCfgCfaUfgUfuGfsasAf | 97 | usUfscAfaCfaUfgCfgGfaUfscsUfscsCfsUfsgsg |
| Clf-92-286 | 81 | GfsasUfcCfgCfaUfgUfuGfsasAf | 97 | usUfscAfaCfaUfgCfgGfaUfscsUfscsCfsUfsgsg |
| Ec-92-286 | 81 | GfsasUfcCfgCfaUfgUfuGfsasAf | 97 | usUfscAfaCfaUfgCfgGfaUfscsUfscsCfsUfsgsg |
| Bt-92-286 | 81 | GfsasUfcCfgCfaUfgUfuGfsasAf | 97 | usUfscAfaCfaUfgCfgGfaUfscsUfscsCfsUfsgsg |
| Fc-92-286 | 81 | GfsasUfcCfgCfaUfgUfuGfsasAf | 97 | usUfscAfaCfaUfgCfgGfaUfscsUfscsCfsUfsgsg |
| Mu-92-286 | 167 | GfsasUfcAfgAfaUfgCfuGfsasAf | 191 | usUfscAfgCfaUfuCfuGfaUfscsUfscsCfsUfsgsg |
| Rn-92-286 | 167 | GfsasUfcAfgAfaUfgCfuGfsasAf | 192 | usUfscAfgCfaUfuCfuGfaUfscsUfscsCfsUfsgsa |
| Mm-92-155 | 82 | CfscsGfcAfuGfuUfgAfasGfsaAf | 98 | usUfscUfuCfaAfcAfuGfcGfsgsAfsusCfsUfscsc |
| Mf-92-155 | 82 | CfscsGfcAfuGfuUfgAfasGfsaAf | 98 | usUfscUfuCfaAfcAfuGfcGfsgsAfsusCfsUfscsc |
| Clf-92-155 | 82 | CfscsGfcAfuGfuUfgAfasGfsaAf | 98 | usUfscUfuCfaAfcAfuGfcGfsgsAfsusCfsUfscsc |
| Ec-92-155 | 82 | CfscsGfcAfuGfuUfgAfasGfsaAf | 98 | usUfscUfuCfaAfcAfuGfcGfsgsAfsusCfsUfscsc |
| Bt-92-155 | 82 | CfscsGfcAfuGfuUfgAfasGfsaAf | 98 | usUfscUfuCfaAfcAfuGfcGfsgsAfsusCfsUfscsc |
| Fc-92-155 | 82 | CfscsGfcAfuGfuUfgAfasGfsaAf | 98 | usUfscUfuCfaAfcAfuGfcGfsgsAfsusCfsUfscsc |
| Mu-92-155 | 168 | CfsasGfaAfuGfcUfgAfaGfsasAf | 193 | usUfscUfuCfaGfcAfuUfcUfsgsAfsusCfsUfscsc |
| Rn-92-155 | 168 | CfsasGfaAfuGfcUfgAfaGfsasAf | 193 | usUfscUfuCfaGfcAfuUfcUfsgsAfsusCfsUfscsc |
| Mm-92-174 | 169 | AfscsGfuAfcAfaCfaCfgGfsasAf | 194 | usUfscCfgUfgUfuGfuAfcGfsusGfscsAfsGfsgsa |
| Mf-92-174 | 169 | AfscsGfuAfcAfaCfaCfgGfsasAf | 194 | usUfscCfgUfgUfuGfuAfcGfsusGfscsAfsGfsgsa |
| Clf-92-174 | 170 | AfscsAfuAfcAfaCfaCfaGfsasAf | 195 | usUfscUfgUfgUfuGfuAfuGfsusAfscsAfsGfsgsa |
| Ec-92-174 | 171 | AfscsGfuAfcAfaCfaCfgGfsasAf | 196 | usUfscCfgUfgUfuGfuAfcGfsusGfscsAfsGfsgsa |
| Bt-92-174 | 172 | AfscsAfuAfcAfaCfaCfgGfsasAf | 197 | usUfscCfgUfgUfuGfuAfuGfsusGfscsAfsAfsgsa |
| Fc-92-174 | 83 | AfscsGfuAfcAfaCfaCfaGfsasAf | 198 | usUfscUfgUfgUfuGfuAfcGfsusAfscsAfsGfsgsa |
| Mu-92-174 | 83 | AfscsGfuAfcAfaCfaCfaGfsasAf | 199 | usUfscUfgUfgUfuGfuAfuGfsusGfscsAfsUfsgsa |
| Rn-92-174 | 170 | AfscsAfuAfcAfaCfaCfaGfsasAf | 200 | usUfscUfgUfgUfuGfuAfuGfsusGfscsAfsCfsgsa |
| Mm-92-221 | 171 | CfscsCfgGfgUfgAfaUfgGfsasAf | 201 | usUfscCfaUfuCfaCfcCfgGfsgsAfsasGfsCfsgsg |
| Mf-92-221 | 171 | CfscsCfgGfgUfgAfaUfgGfsasAf | 201 | usUfscCfaUfuCfaCfcCfgGfsgsAfsasGfsCfsgsg |
| Clf-92-221 | 172 | CfscsCfgGfgUfaAfaUfgGfsgsAf | 202 | usUfscCfaUfuUfaCfcCfgGfsgsAfsasGfsCfsgsg |
| Ec-92-221 | 85 | CfscsAfgGfgUfgAfaUfgGfsasAf | 203 | usUfscCfaUfuCfaCfcCfuGfsgsAfsasUfsCfsgsc |
| Bt-92-221 | 173 | CfscsAfgGfgCfgAfgUfgGfsasAf | 204 | usUfscCfaCfuCfgCfcCfuGfsgsAfsasGfsCfsgsg |
| Fc-92-221 | 85 | CfscsAfgGfgUfgAfaUfgGfsasAf | 101 | usUfscCfaUfuCfaCfcCfuGfsgsAfsasGfsCfsgsg |
| Mu-92-221 | 174 | CfscsCfgGfgUfgAfgUfgGfsasAf | 205 | usUfscCfaCfuCfaCfcCfgGfsgsAfsasGfsCfsgsg |
| Rn-92-221 | 175 | CfscsCfgGfgUfaAfcUfgGfsasAf | 206 | usUfscCfaCfuUfaCfcCfgGfsgsAfsasGfsCfsgsg |
| Mm-92-245 | 81 | GfsasUfcCfgCfaUfgUfuGfsasAf | 105 | usUfscAfaCfaUfgCfgGfaUfscsUfscsCfsusGfsg |
| Mf-92-245 | 81 | GfsasUfcCfgCfaUfgUfuGfsasAf | 105 | usUfscAfaCfaUfgCfgGfaUfscsUfscsCfsusGfsg |
| Clf-92-245 | 81 | GfsasUfcCfgCfaUfgUfuGfsasAf | 105 | usUfscAfaCfaUfgCfgGfaUfscsUfscsCfsusGfsg |
| Ec-92-245 | 81 | GfsasUfcCfgCfaUfgUfuGfsasAf | 105 | usUfscAfaCfaUfgCfgGfaUfscsUfscsCfsusGfsg |
| Bt-92-245 | 81 | GfsasUfcCfgCfaUfgUfuGfsasAf | 105 | usUfscAfaCfaUfgCfgGfaUfscsUfscsCfsusGfsg |
| Fc-92-245 | 81 | GfsasUfcCfgCfaUfgUfuGfsasAf | 105 | usUfscAfaCfaUfgCfgGfaUfscsUfscsCfsusGfsg |
| Mu-92-245 | 167 | GfsasUfcAfgAfaUfgCfuGfsasAf | 207 | usUfscAfgCfaUfuCfuGfaUfscsUfscsCfsusGfsg |
| Rn-92-245 | 167 | GfsasUfcAfgAfaUfgCfuGfsasAf | 208 | usUfscAfgCfaUfuCfuGfaUfscsUfscsCfsusGfsa |
| Mm-92-134 | 82 | CfscsGfcAfuGfuUfgAfasGfsaAf | 106 | usUfscUfuCfaAfcAfuGfcGfsgsAfsusCfsusCfsc |
| Mf-92-134 | 82 | CfscsGfcAfuGfuUfgAfasGfsaAf | 106 | usUfscUfuCfaAfcAfuGfcGfsgsAfsusCfsusCfsc |
| Clf-92-134 | 82 | CfscsGfcAfuGfuUfgAfasGfsaAf | 106 | usUfscUfuCfaAfcAfuGfcGfsgsAfsusCfsusCfsc |
| Ec-92-134 | 82 | CfscsGfcAfuGfuUfgAfasGfsaAf | 106 | usUfscUfuCfaAfcAfuGfcGfsgsAfsusCfsusCfsc |
| Bt-92-134 | 82 | CfscsGfcAfuGfuUfgAfasGfsaAf | 106 | usUfscUfuCfaAfcAfuGfcGfsgsAfsusCfsusCfsc |
| Fc-92-134 | 82 | CfscsGfcAfuGfuUfgAfasGfsaAf | 106 | usUfscUfuCfaAfcAfuGfcGfsgsAfsusCfsusCfsc |
| Mu-92-134 | 168 | CfsasGfaAfuGfcUfgAfaGfsasAf | 209 | usUfscUfuCfaGfcAfuUfcUfsgsAfsusCfsusCfsc |
| Rn-92-134 | 168 | CfsasGfaAfuGfcUfgAfaGfsasAf | 209 | usUfscUfuCfaGfcAfuUfcUfsgsAfsusCfsusCfsc |
| Mm-92-225 | 169 | AfscsGfuAfcAfaCfaCfgGfsasAf | 210 | usUfscCfgUfgUfuGfuAfcGfsusGfscsAfsgsGfsa |
| Mf-92-225 | 169 | AfscsGfuAfcAfaCfaCfgGfsasAf | 210 | usUfscCfgUfgUfuGfuAfcGfsusGfscsAfsgsGfsa |
| Clf-92-225 | 170 | AfscsAfuAfcAfaCfaCfaGfsasAf | 211 | usUfscUfgUfgUfuGfuAfuGfsusAfscsAfsgsGfsa |
| Ec-92-225 | 171 | AfscsGfuAfcAfaCfaCfgGfsasAf | 212 | usUfscCfgUfgUfuGfuAfcGfsusGfscsAfsgsGfsa |
| Bt-92-225 | 172 | AfscsAfuAfcAfaCfaCfgGfsasAf | 213 | usUfscCfgUfgUfuGfuAfuGfsusGfscsAfsasGfsa |
| Fc-92-225 | 83 | AfscsGfuAfcAfaCfaCfaGfsasAf | 214 | usUfscUfgUfgUfuGfuAfcGfsusAfscsAfsgsGfsa |
| Mu-92-225 | 83 | AfscsGfuAfcAfaCfaCfaGfsasAf | 215 | usUfscUfgUfgUfuGfuAfuGfsusGfscsAfsusGfsa |
| Rn-92-225 | 170 | AfscsAfuAfcAfaCfaCfaGfsasAf | 216 | usUfscUfgUfgUfuGfuAfuGfsusGfscsAfscsGfsa |
| Mm-92-171 | 171 | CfscsCfgGfgUfgAfaUfgGfsasAf | 217 | usUfscCfaUfuCfaCfcCfgGfsgsAfsasGfscsGfsg |
| Mf-92-171 | 171 | CfscsCfgGfgUfgAfaUfgGfsasAf | 217 | usUfscCfaUfuCfaCfcCfgGfsgsAfsasGfscsGfsg |
| Clf-92-171 | 172 | CfscsCfgGfgUfaAfaUfgGfsgsAf | 218 | usUfscCfaUfuUfaCfcCfgGfsgsAfsasGfscsGfsg |
| Ec-92-171 | 85 | CfscsAfgGfgUfgAfaUfgGfsasAf | 219 | usUfscCfaUfuCfaCfcCfuGfsgsAfsasUfscsGfsc |
| Bt-92-171 | 173 | CfscsAfgGfgCfgAfgUfgGfsasAf | 220 | usUfscCfaCfuCfgCfcCfuGfsgsAfsasGfscsGfsg |
| Fc-92-171 | 85 | CfscsAfgGfgUfgAfaUfgGfsasAf | 109 | usUfscCfaUfuCfaCfcCfuGfsgsAfsasGfscsGfsg |
| Mu-92-171 | 174 | CfscsCfgGfgUfgAfgUfgGfsasAf | 221 | usUfscCfaCfuCfaCfcCfgGfsgsAfsasGfscsGfsg |
| Rn-92-171 | 175 | CfscsCfgGfgUfaAfcUfgGfsasAf | 222 | usUfscCfaGfuUfaCfcCfgGfsgsAfsasGfscsGfsg |
| Mm-81-253 | 121 | CfcAfgGfaGfaUfcCfgCfaUfgUfuGfaAfsdTdT | 145 | usUfscAfaCfaUfgCfgGfaUfcUfcCfuGfgsdTdT |
| Mf-81-253 | 121 | CfcAfgGfaGfaUfcCfgCfaUfgUfuGfaAfsdTdT | 145 | usUfscAfaCfaUfgCfgGfaUfcUfcCfuGfgsdTdT |
| Clf-81-253 | 121 | CfcAfgGfaGfaUfcCfgCfaUfgUfuGfaAfsdTdT | 145 | usUfscAfaCfaUfgCfgGfaUfcUfcCfuGfgsdTdT |
| Ec-81-253 | 121 | CfcAfgGfaGfaUfcCfgCfaUfgUfuGfaAfsdTdT | 145 | usUfscAfaCfaUfgCfgGfaUfcUfcCfuGfgsdTdT |
| Bt-81-253 | 121 | CfcAfgGfaGfaUfcCfgCfaUfgUfuGfaAfsdTdT | 145 | usUfscAfaCfaUfgCfgGfaUfcUfcCfuGfgsdTdT |
| Fc-81-253 | 121 | CfcAfgGfaGfaUfcCfgCfaUfgUfuGfaAfsdTdT | 145 | usUfscAfaCfaUfgCfgGfaUfcUfcCfuGfgsdTdT |
| Mu-81-253 | 237 | CfcAfgGfaGfaUfcAfgAfaUfgCfuGfaAfsdTdT | 282 | usUfscAfgCfaUfuCfuGfaUfcUfcCfuGfgsdTdT |
| Rn-81-253 | 238 | UfcAfgGfaGfaUfcAfgAfaUfgCfuGfaAfsdTdT | 283 | usUfscAfgCfaUfuCfuGfaUfcUfcCfuGfasdTdT |
| Mm-81-277 | 122 | GfgAfgAfuCfcGfcAfuGfuUfgAfaGfaAfsdTdT | 146 | usUfscUfuCfaAfcAfuGfcGfgAfuCfuCfcsdTdT |
| Mf-81-277 | 122 | GfgAfgAfuCfcGfcAfuGfuUfgAfaGfaAfsdTdT | 146 | usUfscUfuCfaAfcAfuGfcGfgAfuCfuCfcsdTdT |
| Clf-81-277 | 122 | GfgAfgAfuCfcGfcAfuGfuUfgAfaGfaAfsdTdT | 146 | usUfscUfuCfaAfcAfuGfcGfgAfuCfuCfcsdTdT |
| Ec-81-277 | 122 | GfgAfgAfuCfcGfcAfuGfuUfgAfaGfaAfsdTdT | 146 | usUfscUfuCfaAfcAfuGfcGfgAfuCfuCfcsdTdT |
| Bt-81-277 | 122 | GfgAfgAfuCfcGfcAfuGfuUfgAfaGfaAfsdTdT | 146 | usUfscUfuCfaAfcAfuGfcGfgAfuCfuCfcsdTdT |
| Fc-81-277 | 122 | GfgAfgAfuCfcGfcAfuGfuUfgAfaGfaAfsdTdT | 146 | usUfscUfuCfaAfcAfuGfcGfgAfuCfuCfcsdTdT |
| Mu-81-277 | 239 | GfgAfgAfuCfaGfaAfuGfcUfgAfaGfaAfsdTdT | 284 | usUfscUfuCfaGfcAfuUfcUfgAfuCfuCfcsdTdT |
| Rn-81-277 | 239 | GfgAfgAfuCfaGfaAfuGfcUfgAfaGfaAfsdTdT | 284 | usUfscUfuCfaGfcAfuUfcUfgAfuCfuCfcsdTdT |
| Mm-81-244 | 243 | UfcCfuGfcAfcGfuAfcAfaCfaCfgGfaAfsdTdT | 288 | usUfscCfgUfgUfuGfuAfcGfuGfcAfgGfasdTdT |
| Mf-81-244 | 243 | UfcCfuGfcAfcGfuAfcAfaCfaCfgGfaAfsdTdT | 288 | usUfscCfgUfgUfuGfuAfcGfuGfcAfgGfasdTdT |
| Clf-81-244 | 242 | UfcCfuGfuAfcAfuAfcAfaCfaCfaGfaAfsdTdT | 287 | usUfscUfgUfgUfuGfuAfuGfuAfcAfgGfasdTdT |
| Ec-81-244 | 243 | UfcCfuGfcAfcGfuAfcAfaCfaCfgGfaAfsdTdT | 288 | usUfscCfgUfgUfuGfuAfcGfuGfcAfgGfasdTdT |
| Bt-81-244 | 245 | UfcUfuGfcAfcAfuAfcAfaCfaCfgGfaAfsdTdT | 290 | usUfscCfgUfgUfuGfuAfuGfuGfcAfaGfasdTdT |
| Fc-81-244 | 244 | UfcCfuGfuAfcGfuAfcAfaCfaCfaGfaAfsdTdT | 289 | usUfscUfgUfgUfuGfuAfcGfuAfcAfgGfasdTdT |
| Mu-81-244 | 240 | UfcAfuGfcAfcAfuAfcAfaCfaCfaGfaAfsdTdT | 285 | usUfscUfgUfgUfuGfuAfuGfuGfcAfuGfasdTdT |
| Rn-81-244 | 241 | UfcGfuGfcAfcAfuAfcAfaCfaCfaGfaAfsdTdT | 286 | usUfscUfgUfgUfuGfuAfuGfuGfcAfcGfasdTdT |
| Mm-81-247 | 250 | CfcGfcUfuCfcCfgGfgUfgAfaUfgGfaAfsdTdT | 295 | usUfscCfaUfuCfaCfcCfgGfgAfaGfcGfgsdTdT |
| Mf-81-247 | 250 | CfcGfcUfuCfcCfgGfgUfgAfaUfgGfaAfsdTdT | 295 | usUfscCfaUfuCfaCfcCfgGfgAfaGfcGfgsdTdT |
| Clf-81-247 | 248 | CfcGfcUfuCfcCfgGfgUfaAfaUfgGfgAfsdTdT | 293 | usUfscCfaUfuUfaCfcCfgGfgAfaGfcGfgsdTdT |
| Ec-81-247 | 249 | GfcGfaUfuCfcAfgGfgUfgAfaUfgGfaAfsdTdT | 294 | usUfscCfaUfuCfaCfcCfuGfgAfaUfcGfcsdTdT |
| Bt-81-247 | 251 | CfcGfcUfuCfcAfgGfgCfgAfgUfgGfaAfsdTdT | 296 | usUfscCfaCfuCfgCfcCfuGfgAfaGfcGfgsdTdT |
| Fc-81-247 | 125 | CfcGfcUfuCfcAfgGfgUfgAfaUfgGfaAfsdTdT | 149 | usUfscCfaUfuCfaCfcCfuGfgAfaGfcGfgsdTdT |
| Mu-81-247 | 246 | CfcGfcUfuCfcCfgGfgUfgAfgUfgGfaAfsdTdT | 291 | usUfscCfaCfuCfaCfcCfgGfgAfaGfcGfgsdTdT |
| Rn-81-247 | 247 | CfcGfcUfuCfcCfgGfgUfaAfcUfgGfaAfsdTdT | 292 | usUfscCfaGfuUfaCfcCfgGfgAfaGfcGfgsdTdT |
| Mm-85-173 | 129 | CfcAfgGfaGfaUfcCfgCfaUfgUfuGfaAfsusu | 153 | usUfscAfaCfaUfgCfgGfaUfcUfcCfuGfgsusu |
| Mf-85-173 | 129 | CfcAfgGfaGfaUfcCfgCfaUfgUfuGfaAfsusu | 153 | usUfscAfaCfaUfgCfgGfaUfcUfcCfuGfgsusu |
| Clf-85-173 | 129 | CfcAfgGfaGfaUfcCfgCfaUfgUfuGfaAfsusu | 153 | usUfscAfaCfaUfgCfgGfaUfcUfcCfuGfgsusu |
| Ec-85-173 | 129 | CfcAfgGfaGfaUfcCfgCfaUfgUfuGfaAfsusu | 153 | usUfscAfaCfaUfgCfgGfaUfcUfcCfuGfgsusu |
| Bt-85-173 | 129 | CfcAfgGfaGfaUfcCfgCfaUfgUfuGfaAfsusu | 153 | usUfscAfaCfaUfgCfgGfaUfcUfcCfuGfgsusu |
| Fc-85-173 | 129 | CfcAfgGfaGfaUfcCfgCfaUfgUfuGfaAfsusu | 153 | usUfscAfaCfaUfgCfgGfaUfcUfcCfuGfgsusu |
| Mu-85-173 | 252 | CfcAfgGfaGfaUfcAfgAfaUfgCfuGfaAfsusu | 297 | usUfscAfgCfaUfuCfuGfaUfcUfcCfuGfgsusu |
| Rn-85-173 | 253 | UfcAfgGfaGfaUfcAfgAfaUfgCfuGfaAfsusu | 298 | usUfscAfgCfaUfuCfuGfaUfcUfcCfuGfasusu |
| Mm-85-163 | 130 | GfgAfgAfuCfcGfcAfuGfuUfgAfaGfaAfsusu | 154 | usUfscUfuCfaAfcAfuGfcGfgAfuCfuCfcsusu |
| Mf-85-163 | 130 | GfgAfgAfuCfcGfcAfuGfuUfgAfaGfaAfsusu | 154 | usUfscUfuCfaAfcAfuGfcGfgAfuCfuCfcsusu |
| Clf-85-163 | 130 | GfgAfgAfuCfcGfcAfuGfuUfgAfaGfaAfsusu | 154 | usUfscUfuCfaAfcAfuGfcGfgAfuCfuCfcsusu |
| Ec-85-163 | 130 | GfgAfgAfuCfcGfcAfuGfuUfgAfaGfaAfsusu | 154 | usUfscUfuCfaAfcAfuGfcGfgAfuCfuCfcsusu |
| Bt-85-163 | 130 | GfgAfgAfuCfcGfcAfuGfuUfgAfaGfaAfsusu | 154 | usUfscUfuCfaAfcAfuGfcGfgAfuCfuCfcsusu |
| Fc-85-163 | 130 | GfgAfgAfuCfcGfcAfuGfuUfgAfaGfaAfsusu | 154 | usUfscUfuCfaAfcAfuGfcGfgAfuCfuCfcsusu |
| Mu-85-163 | 254 | GfgAfgAfuCfaGfaAfuGfcUfgAfaGfaAfsusu | 299 | usUfscUfuCfaGfcAfuUfcUfgAfuCfuCfcsusu |
| Rn-85-163 | 254 | GfgAfgAfuCfaGfaAfuGfcUfgAfaGfaAfsusu | 299 | usUfscUfuCfaGfcAfuUfcUfgAfuCfuCfcsusu |
| Mm-85-231 | 258 | UfcCfuGfcAfcGfuAfcAfaCfaCfgGfaAfsusu | 303 | usUfscCfgUfgUfuGfuAfcGfuGfcAfgGfasusu |
| Mf-85-231 | 258 | UfcCfuGfcAfcGfuAfcAfaCfaCfgGfaAfsusu | 303 | usUfscCfgUfgUfuGfuAfcGfuGfcAfgGfasusu |
| Clf-85-231 | 257 | UfcCfuGfuAfcAfuAfcAfaCfaCfaGfaAfsusu | 302 | usUfscUfgUfgUfuGfuAfuGfuAfcAfgGfasusu |
| Ec-85-231 | 258 | UfcCfuGfcAfcGfuAfcAfaCfaCfgGfaAfsusu | 303 | usUfscCfgUfgUfuGfuAfcGfuGfcAfgGfasusu |
| Bt-85-231 | 260 | UfcUfuGfcAfcAfuAfcAfaCfaCfgGfaAfsusu | 305 | usUfscCfgUfgUfuGfuAfuGfuGfcAfaGfasusu |
| Fc-85-231 | 259 | UfcCfuGfuAfcGfuAfcAfaCfaCfaGfaAfsusu | 304 | usUfscUfgUfgUfuGfuAfcGfuAfcAfgGfasusu |
| Mu-85-231 | 255 | UfcAfuGfcAfcAfuAfcAfaCfaCfaGfaAfsusu | 300 | usUfscUfgUfgUfuGfuAfuGfuGfcAfuGfasusu |
| Rn-85-231 | 256 | UfcGfuGfcAfcAfuAfcAfaCfaCfaGfaAfsusu | 301 | usUfscUfgUfgUfuGfuAfuGfuGfcAfcGfasusu |
| Mm-85-300 | 265 | CfcGfcUfuCfcCfgGfgUfgAfaUfgGfaAfsusu | 310 | usUfscCfaUfuCfaCfcCfgGfgAfaGfcGfgsusu |
| Mf-85-300 | 265 | CfcGfcUfuCfcCfgGfgUfgAfaUfgGfaAfsusu | 310 | usUfscCfaUfuCfaCfcCfgGfgAfaGfcGfgsusu |
| Clf-85-300 | 263 | CfcGfcUfuCfcCfgGfgUfaAfaUfgGfgAfsusu | 308 | usUfscCfaUfuUfaCfcCfgGfgAfaGfcGfgsusu |
| Ec-85-300 | 264 | GfcGfaUfuCfcAfgGfgUfgAfaUfgGfaAfsusu | 309 | usUfscCfaUfuCfaCfcCfuGfgAfaUfcGfcsusu |
| Bt-85-300 | 266 | CfcGfcUfuCfcAfgGfgCfgAfgUfgGfaAfsusu | 311 | usUfscCfaCfuCfgCfcCfuGfgAfaGfcGfgssusu |
| Fc-85-300 | 133 | CfcGfcUfuCfcAfgGfgUfgAfaUfgGfaAfsusu | 157 | usUfscCfaUfuCfaCfcCfuGfgAfaGfcGfgsusu |
| Mu-85-300 | 261 | CfcGfcUfuCfcCfgGfgUfgAfgUfgGfaAfsusu | 306 | usUfscCfaCfuCfaCfcCfgGfgAfaGfcGfgsusu |
| Rn-85-300 | 262 | CfcGfcUfuCfcCfgGfgUfaAfcUfgGfaAfsusu | 307 | usUfscCfaGfuUfaCfcCfgGfgAfaGfcGfgsusu |
| Mm-87-193 | 137 | cscsagGfaGfAfUfccgcauguugaasusu | 161 | usUfscaaCfaugcggaUfcUfccuggsusu |
| Mf-87-193 | 137 | cscsagGfaGfAfUfccgcauguugaasusu | 161 | usUfscaaCfaugcggaUfcUfccuggsusu |
| Clf-87-193 | 137 | cscsagGfaGfAfUfccgcauguugaasusu | 161 | usUfscaaCfaugcggaUfcUfccuggsusu |
| Ec-87-193 | 137 | cscsagGfaGfAfUfccgcauguugaasusu | 161 | usUfscaaCfaugcggaUfcUfccuggsusu |
| Bt-87-193 | 137 | cscsagGfaGfAfUfccgcauguugaasusu | 161 | usUfscaaCfaugcggaUfcUfccuggsusu |
| Fc-87-193 | 137 | cscsagGfaGfAfUfccgcauguugaasusu | 161 | usUfscaaCfaugcggaUfcUfccuggsusu |
| Mu-87-193 | 267 | cscsagGfaGfAfUfcagaaugcugaasusu | 312 | usUfscagCfauucugaUfcUfccuggsusu |
| Rn-87-193 | 268 | uscsagGfaGfAfUfcagaaugcugaasusu | 313 | usUfscagCfauucugaUfcUfccugasusu |
| Mm-87-156 | 138 | gsgsagAfuCfCfGfcauguugaagaasusu | 162 | usUfscuuCfaacaugcGfgAfucuccsusu |
| Mf-87-156 | 138 | gsgsagAfuCfCfGfcauguugaagaasusu | 162 | usUfscuuCfaacaugcGfgAfucuccsusu |
| Clf-87-156 | 138 | gsgsagAfuCfCfGfcauguugaagaasusu | 162 | usUfscuuCfaacaugcGfgAfucuccsusu |
| Ec-87-156 | 138 | gsgsagAfuCfCfGfcauguugaagaasusu | 162 | usUfscuuCfaacaugcGfgAfucuccsusu |
| Bt-87-156 | 138 | gsgsagAfuCfCfGfcauguugaagaasusu | 162 | usUfscuuCfaacaugcGfgAfucuccsusu |
| Fc-87-156 | 138 | gsgsagAfuCfCfGfcauguugaagaasusu | 162 | usUfscuuCfaacaugcGfgAfucuccsusu |
| Mu-87-156 | 269 | gsgsagAfuCfAfGfaaugcugaagaasusu | 314 | usUfscuuCfagcauucUfgAfucuccsusu |
| Rn-87-156 | 269 | gsgsagAfuCfAfGfaaugcugaagaasusu | 314 | usUfscuuCfagcauucUfgAfucuccsusu |
| Mm-87-230 | 273 | uscscuGfcAfCfGfuacaacacggaasusu | 318 | usUfsccgUfguuguacGfuGfcaggasusu |
| Mf-87-230 | 273 | uscscuGfcAfCfGfuacaacacggaasusu | 318 | usUfsccgUfguuguacGfuGfcaggasusu |
| Clf-87-230 | 272 | uscscuGfuAfCfAfuacaacacagaasusu | 317 | usUfscugUfguuguauGfuAfcaggasusu |
| Ec-87-230 | 273 | uscscuGfcAfCfGfuacaacacggaasusu | 318 | usUfsccgUfguuguacGfuGfcaggasusu |
| Bt-87-230 | 275 | uscsuuGfcAfCfAfuacaacacggaasusu | 320 | usUfsccgUfguuguauGfuGfcaagasusu |
| Fc-87-230 | 274 | uscscuGfuAfCfGfuacaacacagaasusu | 319 | usUfscugUfguuguacGfuAfcaggasusu |
| Mu-87-230 | 270 | uscsauGfcAfCfAfuacaacacagaasusu | 315 | usUfscugUfguuguauGfuGfcaugasusu |
| Rn-87-230 | 271 | uscsguGfcAfCfAfuacaacacagaasusu | 316 | usUfscugUfguuguauGfuGfcacgasusu |
| Mm-87-235 | 280 | cscsgcUfuCfCfCfgggugaauggaasusu | 325 | usUfsccaUfucacccgGfgAfagcggsusu |
| Mf-87-235 | 280 | cscsgcUfuCfCfCfgggugaauggaasusu | 325 | usUfsccaUfucacccgGfgAfagcggsusu |
| Clf-87-235 | 278 | cscsgcUfuCfCfCfggguaaaugggasusu | 323 | usUfsccaUfuuacccgGfgAfagcggsusu |
| Ec-87-235 | 279 | gscsgaUfuCfCfAfgggugaauggaasusu | 324 | usUfsccaUfucacccuGfgAfaucgcsusu |
| Bt-87-235 | 281 | cscsgcUfuCfCfAfgggcgaguggaasusu | 326 | usUfsccaCfucgcccuGfgAfagcggsusu |
| Fc-87-235 | 141 | cscsgcUfuCfCfAfgggugaauggaasusu | 165 | usUfsccaUfucacccuGfgAfagcggsusu |
| Mu-87-235 | 276 | cscsgcUfuCfCfCfgggugaguggaasusu | 321 | usUfsccaCfucacccgGfgAfagcggsusu |
| Rn-87-235 | 277 | cscsgcUfuCfCfCfggguaacuggaasusu | 322 | usUfsccaGfuuacccgGfgAfagcggsusu |

TABLE 10
Un-modified sense and antisense strand sequences of non-human mammalian DBH siNA agents.

| | Nucleotide | SEQ | | | | | |
|---|---|---|---|---|---|---|---|
| | GenBank Accession | ID | | | SEQ | | |
| Duplex name | No | No. | Sense sequence 5′ to 3′ | Range | ID No. | Antisense sequence 5′ to 3′ | Range |
| Mm-57-253 | NM_001278345.2 | 113 | CCAGGAGAUCCGCAUGUUGAA | 1341-1361 | 327 | UUCAACAUGCGGAUCUCCUGG | 1361-1341 |
| Mf-57-253 | XM_005580497.3 | 113 | CCAGGAGAUCCGCAUGUUGAA | 1350-1370 | 327 | UUCAACAUGCGGAUCUCCUGG | 1370-1350 |
| Clf-57-253 | NM_001005263.1 | 113 | CCAGGAGAUCCGCAUGUUGAA | 1316-1336 | 327 | UUCAACAUGCGGAUCUCCUGG | 1336-1316 |
| Ec-57-253 | NM_001081770.3 | 113 | CCAGGAGAUCCGCAUGUUGAA | 1316-1336 | 327 | UUCAACAUGCGGAUCUCCUGG | 1336-1316 |
| Bt-57-253 | NM_180995.2 | 113 | CCAGGAGAUCCGCAUGUUGAA | 1318-1338 | 327 | UUCAACAUGCGGAUCUCCUGG | 1338-1318 |
| Fc-57-253 | XM_003996036.6 | 113 | CCAGGAGAUCCGCAUGUUGAA | 1814-1834 | 327 | UUCAACAUGCGGAUCUCCUGG | 1834-1814 |
| Mu-57-253 | NM_138942.3 | 223 | CCAGGAGAUCAGAAUGCUGAA | 1353-1373 | 328 | UUCAGCAUUCUGAUCUCCUGG | 1373-1353 |
| Rn-57-253 | NM_013158.3 | 224 | UCAGGAGAUCAGAAUGCUGAA | 1344-1364 | 329 | UUCAGCAUUCUGAUCUCCUGA | 1364-1344 |
| Mm-57-277 | NM_001278345.2 | 114 | GGAGAUCCGCAUGUUGAAGAA | 1353-1373 | 330 | UUCUUCAACAUGCGGAUCUCC | 1373-1353 |
| Mf-57-277 | XM_005580497.3 | 114 | GGAGAUCCGCAUGUUGAAGAA | 1319-1339 | 330 | UUCUUCAACAUGCGGAUCUCC | 1339-1319 |
| Clf-57-277 | NM_001005263.1 | 114 | GGAGAUCCGCAUGUUGAAGAA | 1319-1339 | 330 | UUCUUCAACAUGCGGAUCUCC | 1339-1319 |
| Ec-57-277 | NM_001081770.3 | 114 | GGAGAUCCGCAUGUUGAAGAA | 1321-1341 | 330 | UUCUUCAACAUGCGGAUCUCC | 1341-1321 |
| Bt-57-277 | NM_180995.2 | 114 | GGAGAUCCGCAUGUUGAAGAA | 1817-1837 | 330 | UUCUUCAACAUGCGGAUCUCC | 1837-1817 |
| Fc-57-277 | XM_003996036.6 | 114 | GGAGAUCCGCAUGUUGAAGAA | 1356-1376 | 330 | UUCUUCAACAUGCGGAUCUCC | 1376-1356 |
| Mu-57-277 | NM_138942.3 | 225 | GGAGAUCAGAAUGCUGAAGAA | 1350-1370 | 332 | UUCUUCAGCAUUCUGAUCUCC | 1370-1350 |
| Rn-57-277 | NM_013158.3 | 225 | GGAGAUCAGAAUGCUGAAGAA | 1344-1364 | 332 | UUCUUCAGCAUUCUGAUCUCC | 1364-1344 |
| Mm-57-244 | NM_001278345.2 | 226 | UCCUGCACGUACAACACGGAA | 1402-1422 | 336 | UUCCGUGUUGUACGUGCAGGA | 1422-1402 |
| Mf-57-244 | XM_005580497.3 | 226 | UCCUGCACGUACAACACGGAA | 1411-1431 | 336 | UUCCGUGUUGUACGUGCAGGA | 1431-1411 |
| Clf-57-244 | NM_001005263.1 | 227 | UCCUGUACAUACAACACAGAA | 1377-1397 | 335 | UUCUGUGUUGUAUGUACAGGA | 1397-1377 |
| Ec-57-244 | NM_001081770.3 | 228 | UCCUGCACGUACAACACGGAA | 1377-1397 | 338 | UUCCGUGUUGUACGUGCAGGA | 1397-1377 |
| Bt-57-244 | NM_180995.2 | 229 | UCUUGCACAUACAACACGGAA | 1379-1399 | 339 | UUCCGUGUUGUAUGUGCAAGA | 1397-1377 |
| Fc-57-244 | XM_003996036.6 | 230 | UCCUGUACGUACAACACAGAA | 1875-1895 | 337 | UUCUGUGUUGUACGUACAGGA | 1895-1875 |
| Mu-57-244 | NM_138942.3 | 231 | UCAUGCACAUACAACACAGAA | 1414-1434 | 333 | UUCUGUGUUGUAUGUGCAUGA | 1434-1414 |
| Rn-57-244 | NM_013158.3 | 232 | UCGUGCACAUACAACACAGAA | 1408-1428 | 334 | UUCUGUGUUGUAUGUGCACGA | 1428-1408 |
| Mm-57-247 | NM_001278345.2 | 233 | CCGCUUCCCGGGUGAAUGGAA | 1722-1742 | 344 | UUCCAUUCACCCGGGAAGCGG | 1742-1722 |
| Mf-57-247 | XM_005580497.3 | 233 | CCGCUUCCCGGGUGAAUGGAA | 1731-1751 | 344 | UUCCAUUCACCCGGGAAGCGG | 1751-1731 |
| Clf-57-247 | NM_001005263.1 | 234 | CCGCUUCCCGGGUAAAUGGGA | 1745-1765 | 342 | UCCCAUUUACCCGGGAAGCGG | 1765-1745 |
| Ec-57-247 | NM_001081770.3 | 235 | GCGAUUCCAGGGUGAAUGGAA | 1697-1717 | 343 | UUCCAUUCACCCUGGAAUCGC | 1717-1697 |
| Bt-57-247 | NM_180995.2 | 236 | CCGCUUCCAGGGCGAGUGGAA | 1699-1719 | 345 | UUCCACUCGCCCUGGAAGCGG | 1719-1699 |
| Fc-57-247 | XM_003996036.6 | 117 | CCGCUUCCAGGGUGAAUGGAA | 2195-2215 | 331 | UUCCAUUCACCCUGGAAGCGG | 2215-2195 |
| Mu-57-247 | NM_138942.3 | 237 | CCGCUUCCCGGGUGAGUGGAA | 1734-1754 | 340 | UUCCACUCACCCGGGAAGCGG | 1754-1734 |
| Rn-57-247 | NM_013158.3 | 238 | CCGCUUCCCGGGUAACUGGAA | 1728-1748 | 341 | UUCCAGUUACCCGGGAAGCGG | 1748-1728 |

In an embodiment, siNAs were designed, synthesized, and prepared using methods known in the art. Briefly, siNA sequences were synthesized on a 0.2 μmol scale with phosphoramidite chemistry on solid supports. Ancillary synthesis reagents and standard phosphoramidite monomers were procured from commercial suppliers or procured using custom synthesis from various CMOs.

In an embodiment, phosphorothioate linkages were generated using a 100 mM solution of 3-((Dimethylamino-methylidene) amino)-3H-1,2,4-dithiazole-3-thione (DDTT), in anhydrous acetonitrile/pyridine (9:1 v/v). Oxidation time was 5 minutes. All sequences were synthesized with final removal of the DMT group (“DMT-Off”).

In an embodiment, duplexing of single strands was performed on a liquid handling robot. Sense and antisense single strands were combined in an equimolar ratio to a final concentration of 10 μM in 1×PBS in 96 well plates, the plate sealed, incubated at 100° C. for 10 minutes, and subsequently allowed to return slowly to room temperature over a period of 2-3 hours. The concentration and identity of each duplex was confirmed and then subsequently utilized for in vitro screening assays.

### Example 2—Cell Culture

In an embodiment, human SK-N-SH cells and rat PC-12 cells expressing dopamine-beta-hydroxylase were maintained in a humidified atmosphere of 5% CO₂ at 37° C. SK-N-SH were grown in MEM (Sigma, St. Louis, MO) supplemented with 10% fetal bovine serum (FBS) (Gibco, UK), 25 mM sodium bicarbonate (Merck, Germany) and 25 mM N-2-hydroxyethylpiperazine-N′-2-ethanosulfonic acid (HEPES) (Sigma, St. Louis, MO). PC-12 cells were grown in RPMI1640 (Sigma, St. Louis, MO) supplemented with 10% horse serum and 5% fetal bovine serum (FBS) (Gibco, UK), 25 mM sodium bicarbonate (Merck, Germany) and 25 mM N-2-hydroxyethylpiperazine-N′-2-ethanosulfonic acid (HEPES) (Sigma, St. Louis, MO). The cell culture medium was changed every 2 days, and SK-N-SH cells reached confluence 3-4 days after initial seeding. For subculturing, SK-N-SH cells were dissociated with 0.25% trypsin-ethylenediaminetetraacetic acid (EDTA) (Sigma, St. Louis, MO), split 1:15 or 1:20 and subcultured in a 21-cm² growth area (Sarstedt, Germany). PC-12 cells were sub-cultured 3-4 days, split saturated at 20-40×10⁵ cells/mL and seeded at 5×10⁵ cells/mL in 10 mL; 10 mL of cell medium is added to cells one or two days after split. PC-12 cells were cultured in Nunc Non-treated T75 EasyFlask.

### Example 3—DBH Gene Expression

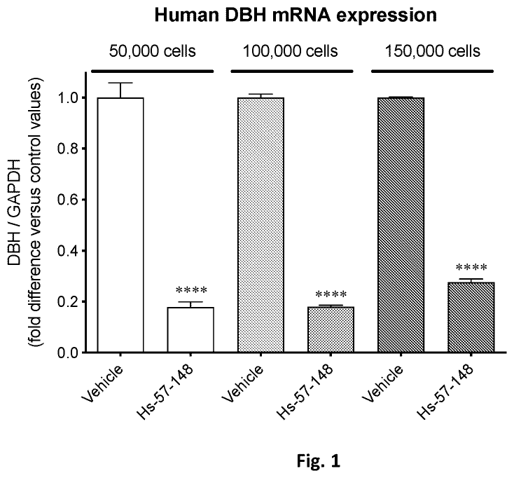

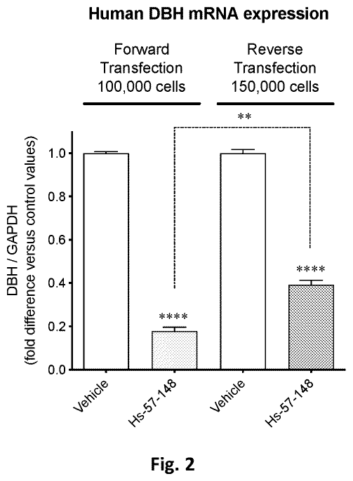

In an embodiment, total RNA was isolated and purified using the SV Total RNA Isolation System (Promega, USA) according to manufacturer's instructions. RNA quality and concentration were verified in the NanoDrop ND1000 Spectrophotometer (Thermo Scientific, USA), and RNA integrity and genomic DNA contamination were evaluated by agarose gel electrophoresis. Total RNA (1 μg) was converted into cDNA using the Maxima Scientific First Strand cDNA Synthesis Kit for RT-qPCR (Thermo Scientific, USA), according to instructions. The following protocol was used: 1^{st} step, 10 min at 25° C.; 2^{nd} step, 15 min at 50° C.; 3^{rd} step, 5 min at 85° C. cDNA was used for qPCR analysis using Maxima SYBR Green qPCR Master Mix (Thermo Scientific, USA) in the StepOnePlus instrument (Applied Biosystems, USA). Primer Assay for dopamine-beta-hydroxylase and for the endogenous control gene GAPDH (Quiagen, Germany) were used. The qPCR reaction was performed in 96-well PCR plates (Sarstedt, Germany) as follows: one cycle of 10 min at 95° C., followed by 40 PCR cycles at 95° C. 15 s and 60° C. 60 s. A melting curve was made immediately after the qPCR, to demonstrate the specificity of the amplification. No template controls were always evaluated for each target gene. Quantification cycle (Cq) values were generated automatically by the StepOnePlus 2.3 Software and the ratio of the target gene was expressed in comparison to the endogenous control gene GAPDH. Real-time PCR efficiencies were found to be between 90% and 110%. SK-N-SH cells express high levels of DBH. Selected siNAs against DBH showed significant efficacy in reducing DBH mRNA expression (FIGS. 1, 2, 3, 4, 5 and 11).

### Example 4—DBH Activity

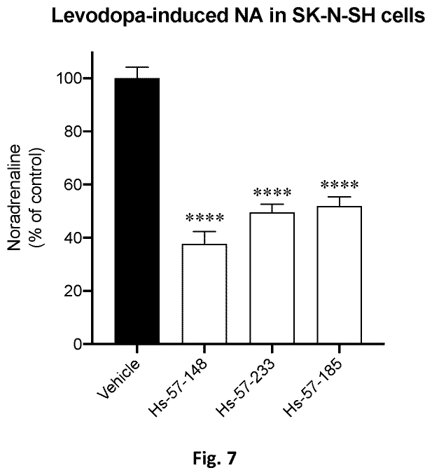

Cells were rinsed twice with cold phosphate-buffered saline (PBS) and pre-incubated for 15 minutes in Hanks media at 37° C. Hanks media had the following composition (in mM): NaCl 140, KCl 5, MgSO₄-7H₂O 0.8, K₂HPO₄ 0.33, KH₂PO₄ 0.44, MgCl²·6H₂O 1.0, CaCl² 0.025, Tris-HCl 9.75, pH 7.4. The reaction was initiated by adding 3 μM L-dihydroxyphenylalanine 8levodopa) plus ascorbic acid (at 1 mM; co-factor) to the Hanks media, for 360 minutes. During the pre-incubation and the incubation, cells were continuously shacked and maintained at 37° C. in a water bath. The reaction was stopped through the rapid removal of the incubation solution through aspiration with a Pasteur pipette, followed by a quick wash with Hanks media. Subsequently, cells were added with 0.2 M perchloric acid and stored at 4° C. for 24 hours. Thereafter, 900 μL of perchloric acid in which the cells were kept was used for the quantification of noradrenaline by means of high-pressure liquid chromatography with electrochemical detection (HPLC-EC). Selected siNAs against DBH showed marked efficacy in reducing DBH activity as evidenced by decreases in noradrenaline synthesis, which is in line with their ability to downregulate DBH mRNA expression (FIGS. 7, 8, 9, 12 and 13).

### Example 7—DBH Protein Expression

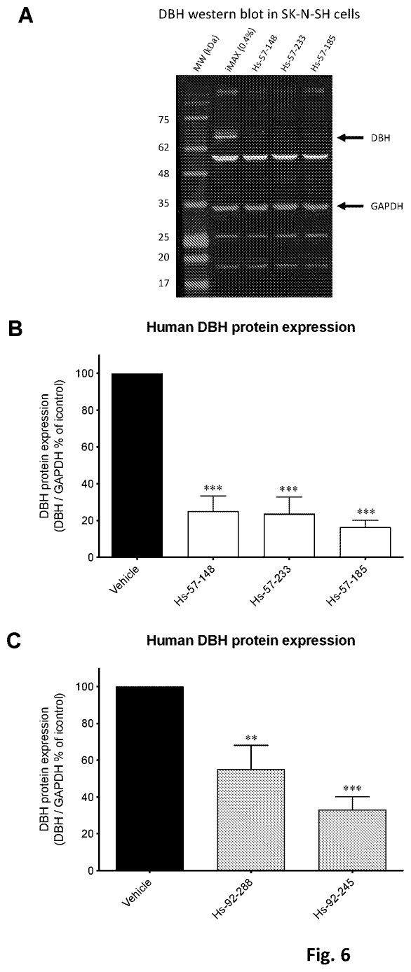

In an embodiment, cells were rinsed twice with cold phosphate-buffered saline (PBS) and incubated with 100 μL RIPA lysis buffer (154 mM NaCl, 65.2 mM TRIZMA base, 1 mM EDTA, 1% NP-40 (IGEPAL), 6 mM sodium deoxycholate) containing protease inhibitors: 1 mM PMSF, 1 μg/mL leupeptine and 1 μg/mL aprotinin; and phosphatase inhibitors: 1 mM Na₃VO₄ and 1 mM NaF. Cells were scraped and briefly sonicated. Equal amounts of total protein (30 μg) were separated on a 10% SDS-polyacrylamide gel and electrotransfered to a nitrocellulose membrane in Tris-Glycine transfer buffer containing 20% methanol. The transblot sheets were blocked in 5% non-fat dry milk in Tris-buffered saline (TBS) for 60 min and then incubated overnight, at 4° C., with the antibodies against dopamine-beta-hydroxylase and GAPDH, diluted in 2.5% non-fat dry milk in TBS-Tween 20 (0.1% vol/vol). The immunoblots were subsequently washed and incubated with fluorescently-labelled secondary antibodies (1:20,000; AlexaFluor 680, Molecular Probes) for 60 min at room temperature (RT) and protected from light. Membranes were washed and imaged by scanning at both 700 nm and 800 nm with an Odyssey Infrared Imaging System (LI-COR Biosciences). The levels of DBH protein expression were evaluated in SK-N-SH cells. Selected siNA against DBH showed significant efficacy in reducing DBH protein expression (FIG. 6).

### Example 8—Stability of Chemically Modified siNAs

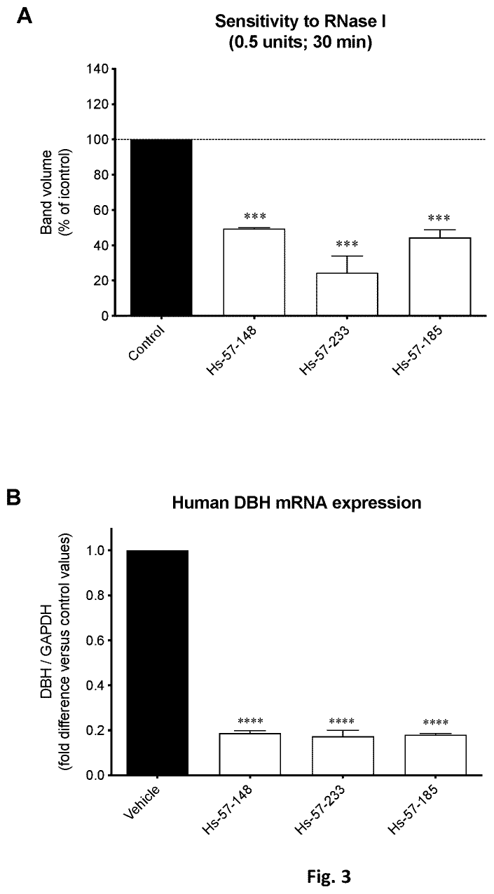

In an embodiment, siNA sequences used in the study were thaw and incubated at 37° C. during up to 30 min with cell serum-free culture medium added with RNase I (0.50 Units). In contrast to non-modified (natural) siNAs, chemically modified siNAs against LAT1 and ASCT2 showed a significant resistance to degradation in culture medium containing RNase I (0.50 Units) for up to 30 min (FIGS. 3, 4 and 11).

### Example 9—Human DBH Gene Silencing of Chemically Modified siNAs

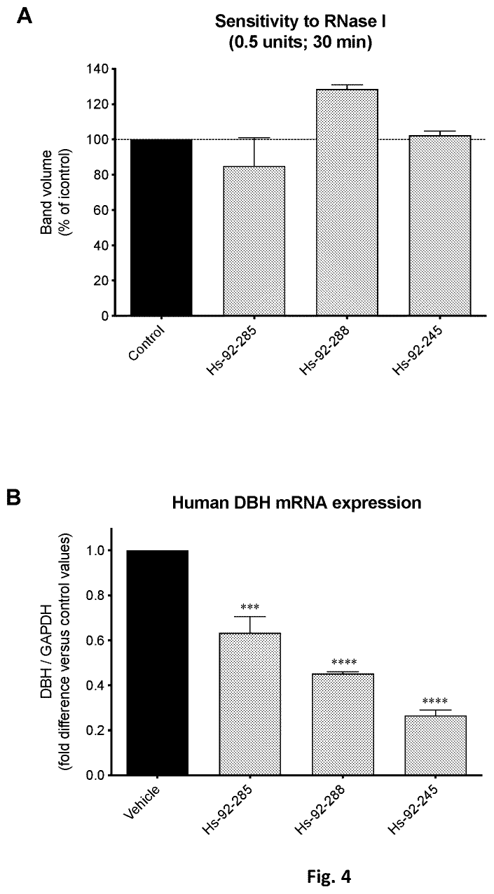

In an embodiment, human SK-N-SH cells were plated in 24-well (Sarstedt, Germany) or 6-well plates (Sarstedt, Germany) or 96-well plates with black walls clear bottom (BD Biosciences, USA) and incubated 24 h under normal growth conditions. siNAs against DBH and transfection agent were diluted at desired concentrations and mixed according to transfection agent manufacturer's instructions. The mixture was incubated 20 min at room temperature for siNA-complex formation, after which it was added to the cells and incubated at 37° C., 5% CO₂. After the incubation period, serum and antibiotic was restored and cells were further incubated at normal conditions for the desired time points until evaluation of DBH activity or DBH mRNA expression (RT-qPCR). Selected chemically un-modified siNAs against human DBH (duplex Hs-57-148-sense SEQ ID No. 31 and antisense SEQ ID No. 55; duplex Hs-57-233-sense SEQ ID No. 32 and antisense SEQ ID No. 56; duplex Hs-57-185-sense SEQ ID No. 33 and antisense SEQ ID No. 57) showed marked efficacy in reducing DBH mRNA expression (FIG. 3), which is in line with their capacity to downregulate DBH protein expression (FIG. 6). Chemically modified siNAs against human DBH, in contrast to un-modified siNAs, showed marked insensitivity to degradation in culture medium containing RNase I (0.50 Units) for up to 30 min, retain their capacity in RISC engagement and markedly downregulate of DBH mRNA expression (FIG. 4).

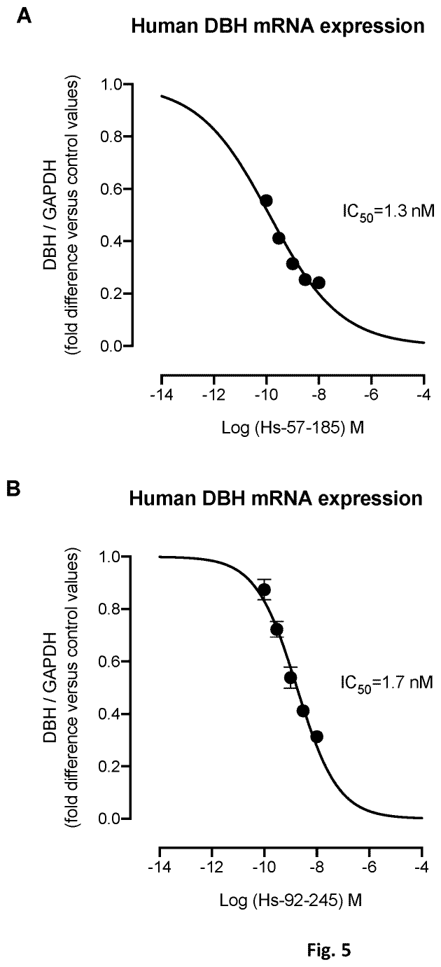

In another embodiment, chemically modified duplexes of the Hs-92 series, such as duplexes Hs-92-285 (sense SEQ ID No. 79 and antisense SEQ ID No. 103), Hs-92-288 (sense SEQ ID No. 80 and antisense SEQ ID No. 104), and Hs-92-245 (sense SEQ ID No. 81 and antisense SEQ ID No. 105), assembled as asymmetric duplexes were found to be insensitive to degradation in culture medium containing RNase I, but markedly downregulate human DBH mRNA expression (FIG. 4). Surprisingly, the efficacy of the asymmetric chemically modified duplex Hs-92-245 (sense SEQ ID No. 81 and antisense SEQ ID No. 105) in downregulate human DBH mRNA expression and human DBH protein expression was not different from that obtained with the symmetric and chemically un-modified duplex Hs-57-185 (sense SEQ ID No. 33 and antisense SEQ ID No. 57) (FIGS. 5 and 6).

### Example 10—Rat DBH Gene Silencing of Chemically Modified siNAs

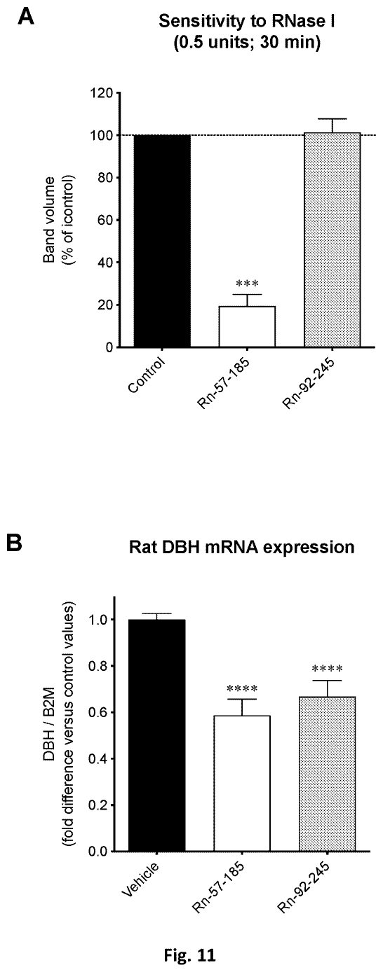

In an embodiment, rat PC-12 cells were cultured in T75 flasks (Sarstedt, Germany) and incubated 24 h under normal growth conditions. siNAs against rat DBH and transfection agent were diluted at desired concentrations and mixed according to transfection agent manufacturer's instructions. The mixture was incubated 20 min at room temperature for siNA-complex formation, after which it was added to the cells and incubated at 37° C., 5% CO₂. After the incubation period, serum and antibiotic was restored and cells were further incubated at normal conditions for the desired time points until evaluation of rat DBH mRNA expression (RT-qPCR) or DBH activity. Selected chemically un-modified siNAs against rat DBH (duplex Rn-57-185-sense SEQ ID No. 224 and antisense SEQ ID No. 299) showed marked efficacy in reducing DBH mRNA expression (FIG. 11). Chemically modified siNAs against rat DBH, in contrast to un-modified siNAs, showed marked insensitivity to degradation in culture medium containing RNase I (0.50 Units) for up to 30 min (FIG. 11A), retain their capacity in RISC engagement and markedly downregulate of DBH mRNA expression (FIG. 11B).

### Example 11—DBH Activity

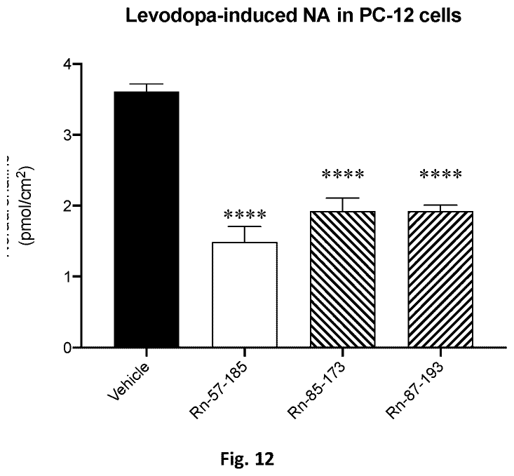

In an embodiment, human SK-N-SH cells and rat PC-12 cells were incubated 24 h under normal growth conditions and siNAs against DBH and transfection agent were diluted at desired concentrations and mixed according to transfection agent manufacturer's instructions. The mixture was incubated 20 min at room temperature for siNA-complex formation, after which it was added to the cells and incubated at 37° C., 5% CO₂. After the incubation period, serum and antibiotic was restored and cells were further incubated at normal conditions for the desired time points until evaluation of DBH activity, as revealed by their ability to convert levodopa to noradrenaline in the presence of ascorbic acid. Selected chemically un-modified siNAs against human DBH (duplex Hs-57-148-sense SEQ ID No. 31 and antisense SEQ ID No. 55; duplex Hs-57-233-sense SEQ ID No. 32 and antisense SEQ ID No. 56; duplex Hs-57-185-sense SEQ ID No. 33 and antisense SEQ ID No. 57) showed marked efficacy in reducing DBH activity (FIG. 7), which is in line with their capacity to downregulate DBH mRNA and DBH protein expression (FIGS. 3 and 6). Similarly, in PC-12 cells, selected chemically un-modified siNAs against rat DBH duplex Rn-57-185 (sense SEQ ID No. 224 and antisense SEQ ID No. 299) showed marked efficacy in reducing DBH activity (FIG. 12), which is in line with their capacity to downregulate DBH mRNA (FIG. 11).

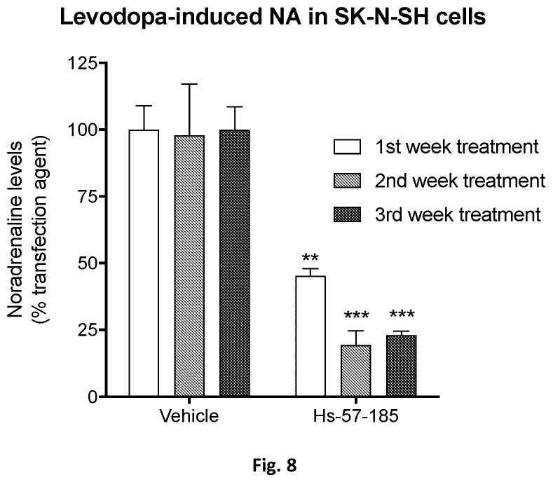

In another embodiment, SK-N-SH cells treated over 3 consecutive weeks with the selected chemically un-modified siNA against human DBH duplex Hs-57-185 (sense SEQ ID No. 33 and antisense SEQ ID No. 57) showed marked and time dependent efficacy in reducing DBH activity (FIG. 8).

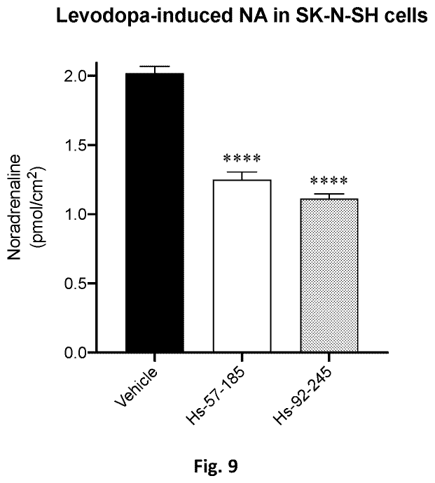

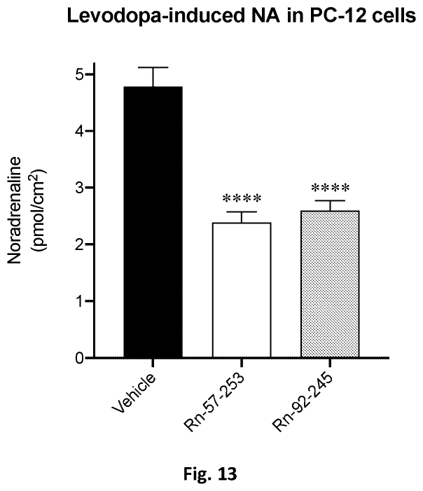

In another embodiment, chemically modified and asymmetric siNAs against human DBH, exemplified here with duplex Hs-92-245 (sense SEQ ID No. 81 and antisense SEQ ID No. 105), in line with that observed for decreases in DBH mRNA and DBH protein expression (FIGS. 4 and 6), showed marked showed marked efficacy in reducing DBH activity in SK-N-SH cells (FIG. 9). Similarly, in PC-12 cells, selected chemically modified siNAs against rat DBH, respectively symmetric duplexes Rn-85-173 (sense SEQ ID No. 253 and antisense SEQ ID No. 298) and Rn-87-193 (sense SEQ ID No. 268 and antisense SEQ ID No. 313), or asymmetric duplexes, such as Rn-92-245 (sense SEQ ID No. 167 and antisense SEQ ID No. 208), showed marked efficacy in reducing DBH activity (FIGS. 12 and 13).

### Example 12—Induction of High Intra Ocular Pressure (Experimental Glaucoma)

Female Wistar Han IGS (Charles River Laboratories, Spain), 150-200 g body weight were used in these experiments. The animals received standard food and water ad libitum and were kept in a 12/12 h light/dark cycle at constant environment temperature (21° C.) and humidity (65%). All experimental protocols involving animals were approved by the competent national authority Direçáo Geral de Alimentaçáo e Veterinária, and by the Animal Ethical Committee of ICBAS (No. 316/2019). All efforts were made to minimize animal suffering and to reduce the number of animals used according to the ARRIVE guidelines.

The IOP was measured in conscious rats using a rebound tonometer specifically designed for rodents (model Tv02, iCare® TonoLab, Finland). As it is a noncontact tonometer, the use of an anesthetic agent is not required. All animals were handled twice daily by the investigators for at least one week before starting any experimental procedure. The animals were identified by tail numbers and had their whiskers cut off to avoid interference with IOP measurements. Two to three days before the experiment, IOPs of both eyes were measured twice daily (mornings and afternoons) for the animals to get acquainted with the procedure. Measurements were made in a quiet environment with a maximum of two investigators present. When measuring the IOP, each animal was restrained with a piece of cloth so it could remain comfortable, calm and immobile. The tonometer was stably attached to the table. The researcher adjusted the animal's position so that the central cornea was aligned with the tip of the TonoLab probe. The distance should be 1-4 mm from the tip of the probe and the cornea. Measure takes place when the operator presses the measurement button. The tip of the probe should contact the central cornea and one value is obtained. Six measurements were made consecutively, which were averaged before the IOP value is displayed in the equipment; this procedure was repeated six times. In case of data disparity, one or two extra measurements were added. To avoid disturbing the animal, a second operator helps in IOP readings.

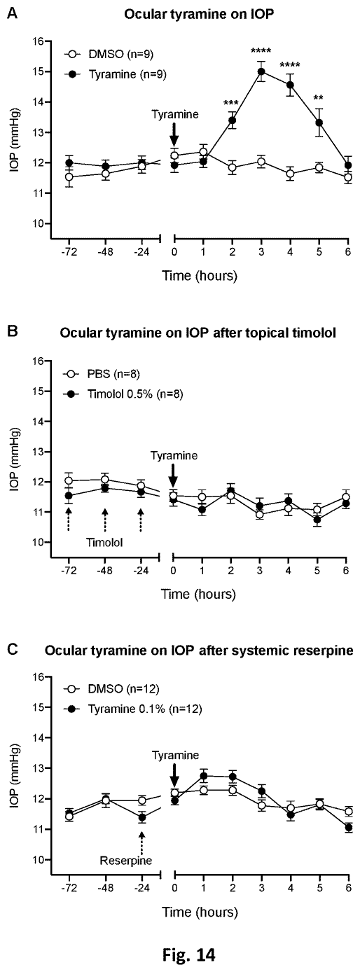

For the first time a rat model of intraocular high IOP induced by topical application of tyramine 0.1% in eye drops (10-μl) using a micropipette was used. Tyramine is an indirect sympathomimetic agent that mobilizes noradrenaline stored in synaptic vesicles of the ocular noradrenergic nerve terminals. Noradrenaline activating adrenergic receptors is responsible for increasing the secretion of aqueous humor and/or decreasing its drainage, leading to increased IOP. Tyramine (Sigma-Aldrich, catalogue number: T90344-5G, lot #BCCC6853) has a low solubility in water or aqueous solutions, so a stock solution of 20 mg tyramine per 1 mL DMSO (tyramine 2% in DMSO) was prepared. Then the tyramine stock solution was diluted to 0.1% in saline solution for topical application in the eye. Controls were made using the tyramine's vehicle up to 5% DMSO (v/v) in saline solution. The increase in IOP caused by tyramine 0.1% was evaluated at 60-min intervals during 6 hours comparing the values obtained before (baseline) and after application of the drug. FIG. 14A shows that topical application of tyramine 0.1% in the eye typically increases the IOP above baseline from the 2^{nd} to the 5^{th} hour following the drug application. The tyramine-induced increase in IOP was prevented by pretreatment with 0.5% timolol applied as eye drops 30 min before the application of tyramine (FIG. 14B). Similarly, pretreatment with reserpine (1 mg/kg, i.p.) 24 hours prior to the eye application of tyramine prevented the tyramine-induced increase in IOP (FIG. 14C).

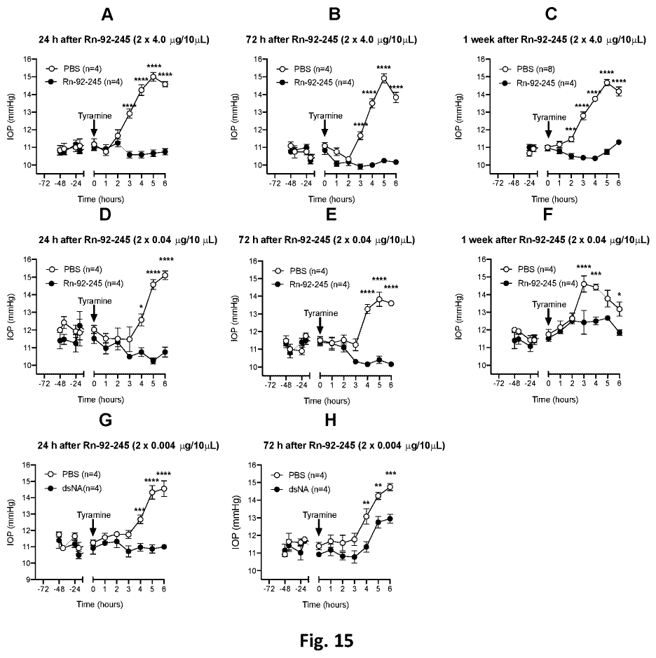

For the evaluation of the effect of anti-DBH siNAs on the tyramine-induced high IOP, selected siNAs were applied topically in 10-μl eye drops twice a day (at 10 and 16 hours) for 1 or 3 days; the right eye (RE) was used to test the effect of the siRNA, while the same amount of vehicle (PBS) was applied in left eye (LE) serving as internal control. The IOP of both eyes was determined using TonoLab measurements immediately before application of the siNA (RE) or vehicle (LE). The preventive effect of selected siNAs was evaluated at 24, 48, 72 hours and/or 1 week after the initial siNA boost by repeating the 6-hour tyramine test. Selected chemically modified siNAs against rat DBH, such as the asymmetric duplex Rn-92-245 (sense SEQ ID No. 167 and antisense SEQ ID No. 208) showed marked efficacy in preventing the tyramine-induced increase of IOP at dose levels of 4.0, 0.04 and 0.004 μg/10 μL applied twice (12 h apart) in the day before the first tyramine (0.1%, ocular application) test at 24 h (A, D, G), 72 h (B, E, H) and/or 1 week (C, F) after the initial rat anti-DBH siNA duplex Rn-92-245 boost (FIG. 15).

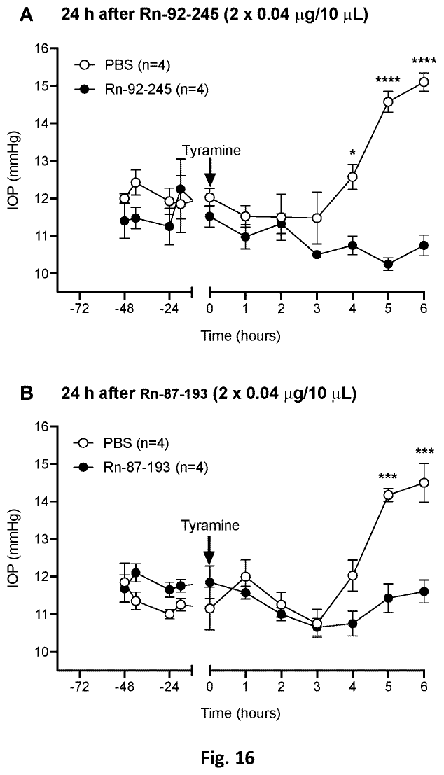

In another embodiment, the chemically modified symmetric duplex Rn-87-193 (sense SEQ ID No. 268 and antisense SEQ ID No. 313) also showed marked efficacy in preventing the tyramine-induced increase of IOP at dose levels of 0.04 μg/10 μL applied twice (12 h apart) in the day before the first tyramine (0.1%, ocular application) test at 24 h after the initial rat anti-DBH siNA duplex boost (FIG. 16).

Additional aspects of the invention will be apparent to those skilled in the art, or may be learned from the practice of the invention. The objects and advantages of the invention may be realized and attained by means of the instrumentalities and combinations particularly pointed out in the appended claims.

The term “comprising” whenever used in this document is intended to indicate the presence of stated features, integers, steps, components, but not to preclude the presence or addition of one or more other features, integers, steps, components or groups thereof.

The disclosure should not be seen in anyway restricted to the embodiments described and a person with ordinary skill in the art will foresee many possibilities to modifications thereof. The above described embodiments are combinable.

The following claims further set out particular embodiments of the disclosure.

### REFERENCES

Ahmed F A, Hegazy K, Chaudhary P, & Sharma S C (2001). Neuroprotective effect of alpha(2) agonist (brimonidine) on adult rat retinal ganglion cells after increased intraocular pressure. Brain Res 913: 133-139.
Bernstein E, Caudy A A, Hammond S M, & Hannon G J (2001). Role for a bidentate ribonuclease in the initiation step of RNA interference. Nature 409: 363-366.
Brummelkamp T R, Bernards R, & Agami R (2002). A system for stable expression of short interfering RNAs in mammalian cells. Science 296: 550-553.
Burke J, Kharlamb A, Shan T, Runde E, Padillo E, Manlapaz C, et al. (1995). Adrenergic and imidazoline receptor-mediated responses to UK-14,304-18 (brimonidine) in rabbits and monkeys. A species difference. Ann N Y Acad Sci 763: 78-95.
Campochiaro P A (2006). Potential applications for RNAi to probe pathogenesis and develop new treatments for ocular disorders. Gene Ther 13: 559-562.
Coleman A L, & Miglior S (2008). Risk factors for glaucoma onset and progression. Surv Ophthalmol 53 Suppl1: S3-10.
Davis B M, Crawley L, Pahlitzsch M, Javaid F, & Cordeiro M F (2016). Glaucoma: the retina and beyond. Acta Neuropathol 132: 807-826.
Davis M E (2009). The first targeted delivery of siRNA in humans via a self-assembling, cyclodextrin polymer-based nanoparticle: from concept to clinic. Mol Pharm 6: 659-668.
Dias N, & Stein C A (2002). Antisense oligonucleotides: basic concepts and mechanisms. Mol Cancer Ther 1: 347-355.
Elbashir S M, Harborth J, Lendeckel W, Yalcin A, Weber K, & Tuschl T (2001). Duplexes of 21-nucleotide RNAs mediate RNA interference in cultured mammalian cells. Nature 411: 494-498.
Elbashir S M, Lendeckel W, & Tuschl T (2001). RNA interference is mediated by 21- and 22-nucleotide RNAs. Genes Dev 15: 188-200.
Fire A, Xu S, Montgomery M K, Kostas S A, Driver S E, & Mello C C (1998). Potent and specific genetic interference by double-stranded RNA in Caenorhabditis elegans. Nature 391: 806-811.
Frezzotti P, Giorgio A, Motolese I, De Leucio A, Iester M, Motolese E, et al. (2014). Structural and functional brain changes beyond visual system in patients with advanced glaucoma. PLoS One 9: e105931.
Frezzotti P, Giorgio A, Toto F, De Leucio A, & De Stefano N (2016). Early changes of brain connectivity in primary open angle glaucoma. Hum Brain Mapp 37: 4581-4596.
Fudemberg S J, Batiste C, & Katz L J (2008). Efficacy, safety, and current applications of brimonidine. Expert Opin Drug Saf 7: 795-799.
Greenfield D S, Liebmann J M, & Ritch R (1997). Brimonidine: a new alpha2-adrenoreceptor agonist for glaucoma treatment. J Glaucoma 6: 250-258.
Gupta S K, Agarwal R, Galpalli N D, Srivastava S, Agrawal S S, & Saxena R (2007). Comparative efficacy of pilocarpine, timolol and latanoprost in experimental models of glaucoma. Methods Find Exp Clin Pharmacol 29: 665-671.
Hannon G J (2002). RNA interference. Nature 418: 244-251.
Harborth J, Elbashir S M, Bechert K, Tuschl T, & Weber K (2001). Identification of essential genes in cultured mammalian cells using small interfering RNAs. J Cell Sci 114: 4557-4565.
Hey J A, Gherezghiher T, & Koss M C (1985). Studies on the mechanism of clonidine-induced mydriasis in the rat. Naunyn Schmiedebergs Arch Pharmacol 328: 258-263.
Hsu W H, Lee P, & Betts D M (1981). Xylazine-induced mydriasis in rats and its antagonism by alpha-adrenergic blocking agents. J Vet Pharmacol Ther 4: 97-101.
Ishikawa M, Yoshitomi T, Zorumski C F, & Izumi Y (2015). Experimentally Induced Mammalian Models of Glaucoma. Biomed Res Int 2015: 281214.
Lee N S, Dohjima T, Bauer G, Li H, Li M-J, Ehsani A, et al. (2001). Expression of small interfering RNAs targeted against HIV-1 rev transcripts in human cells. Nature Biotechnology 19: 500-505.
Lee N S, Dohjima T, Bauer G, Li H, Li M J, Ehsani A, et al. (2002). Expression of small interfering RNAs targeted against HIV-1 rev transcripts in human cells. Nat Biotechnol 20: 500-505.
Lima W F, Prakash T P, Murray H M, Kinberger G A, Li W, Chappell A E, et al. (2012). Single-stranded siRNAs activate RNAi in animals. Cell 150: 883-894.
Martinez T, Gonzalez M V, Roehl I, Wright N, Paneda C, & Jimenez A I (2014). In vitro and in vivo efficacy of SYL040012, a novel siRNA compound for treatment of glaucoma. Mol Ther 22: 81-91.
Miyagishi M, & Taira K (2002). U6 promoter-driven siRNAs with four uridine 3′ overhangs efficiently suppress targeted gene expression in mammalian cells. Nature Biotechnology 19: 497-500.
Nucci C, Martucci A, Cesareo M, Mancino R, Russo R, Bagetta G, et al. (2013). Brain involvement in glaucoma: advanced neuroimaging for understanding and monitoring a new target for therapy. Curr Opin Pharmacol 13: 128-133.
Nykanen A, Haley B, & Zamore P D (2001). ATP requirements and small interfering RNA structure in the RNA interference pathway. Cell 107: 309-321.
Okamura T, Fujioka H, & Ayajiki K (2002). Effects of nipradilol on alpha-adrenoceptor function in ocular arteries. Pharmacology 65: 110-118.
Paddison P J, Caudy A A, Bernstein E, Hannon G J, & Conklin D S S (2002). hort hairpin RNAs (shRNAs) induce sequence-specific silencing in mammalian cells. Genes Dev 16: 948-958.
Paul C P, Good P D, Winer I, & Engelke D R (2002). Effective Expression of Small Interfering RNA in human cells. Nature Biotechnology 19: 505-508.
Prokofyeva E, & Zrenner E (2012). Epidemiology of major eye diseases leading to blindness in Europe: a literature review. Ophthalmic Res 47: 171-188.
Quigley H A (2011). Glaucoma. Lancet 377: 1367-1377.
Rittenhouse K D, & Pollack G M (2000). Pharmacodynamics of beta-blocker modulation of aqueous humor production. Exp Eye Res 70: 429-439.
Seki M, Tanaka T, Matsuda H, Togano T, Hashimoto K, Ueda J, et al. (2005). Topically administered timolol and dorzolamide reduce intraocular pressure and protect retinal ganglion cells in a rat experimental glaucoma model. Br J Ophthalmol 89: 504-507.
Sharp P A (2001). RNA interference—2001. Genes Dev 15: 485-490.
Sui G, Soohoo C, Affar E B, Gay F, Shi Y, Forrester W C, et al. (2002). A DNA vector-based RNAi technology to suppress gene expression in mammalian cells. Proc Natl Acad Sci USA 99: 5515-5520.
Tuschl T, Sharp P A, & Bartel D P (1998). Selection in vitro of novel ribozymes from a partially randomized U2 and U6 snRNA library. EMBO J 17: 2637-2650.
Webers C A, Beckers H J, Nuijts R M, & Schouten J S (2008). Pharmacological management of primary open-angle glaucoma: second-line options and beyond. Drugs Aging 25: 729-759.
Wheeler L A, & Woldemussie E (2001). Alpha-2 adrenergic receptor agonists are neuroprotective in experimental models of glaucoma. Eur J Ophthalmol 11 Suppl 2: S30-35.
Xia H, Mao Q, Paulson H L, & Davidson B L (2002). siRNA-mediated gene silencing in vitro and in vivo. Nat Biotechnol 20: 1006-1010.
Yu J Y, DeRuiter S L, & Turner D L (2002). RNA interference by expression of short-interfering RNAs and hairpin RNAs in mammalian cells. Proc Natl Acad Sci USA 99: 6047-6052.

1. A double stranded short interfering nucleic acid (siNA) agent for use in medicine comprising an antisense ribonucleotide sequence arranged to base-pair to a substantially complementary sense ribonucleotide sequence;
wherein all of the nucleotides of the sense strand and all of the nucleotides of the antisense strand comprise a 2′-O-methyl modification or a 2′-fluoro modification;
wherein both the sense strand and the antisense strand independently comprise at least one phosphorothioate internucleotide linkage;
wherein the antisense ribonucleotide sequence is substantially complementary to a sense ribonucleotide sequence except in up to 6 nucleotides;
wherein the sense strand comprises 15 to 21 nucleotides;
wherein each nucleotide or each of the first 18 contiguous nucleotides in the 5′-3′ direction comprise a modification that is different from the modification of the adjacent nucleotide;
and wherein the first modification in the antisense strand is a 2′-O-methyl modification.

2. (canceled)

3. (canceled)

4. (canceled)

5. (canceled)

6. (canceled)

7. (canceled)

8. (canceled)

9. The double stranded siNA agent according to claim 1, wherein the antisense ribonucleotide sequence is at least 90% identical to the sequences of the following list: SEQ ID No. 89, SEQ ID No. 90, SEQ ID No. 91, SEQ ID No. 93, SEQ ID No. 97, SEQ ID No. 98, SEQ ID No. 99, SEQ ID No. 101, SEQ ID No. 105, SEQ ID No. 106, SEQ ID No. 107, SEQ ID No. 109, SEQ ID No. 145, SEQ ID No. 146, SEQ ID No. 147, SEQ ID No. 149, SEQ ID No. 153, SEQ ID No. 154, SEQ ID No. 155, SEQ ID No. 157, SEQ ID No. 161, SEQ ID No. 162, SEQ ID No. 163, SEQ ID No. 165, SEQ ID No. 166; and
the sense ribonucleotide sequence is at least 90% identical to the sequences of the following list: SEQ ID No. 81, SEQ ID No. 82, SEQ ID No. 83, SEQ ID No. 85, SEQ ID No. 121, SEQ ID No. 122, SEQ ID No. 123, SEQ ID No. 125, SEQ ID No. 129, SEQ ID No. 130, SEQ ID No. 131, SEQ ID No. 133, SEQ ID No. 137, SEQ ID No. 138, SEQ ID No. 139, SEQ ID No. 141, SEQ ID No. 142.

10. The siNA agent for use according to claim 1, wherein the modification of each nucleotide is different from the modification of the adjacent and of the complementary nucleotide;
and wherein the first modification in the antisense strand is a 2′-O-methyl modification.

11. The siNA agent for use according to claim 1, wherein the 3 nucleotides adjacent to 3′ comprise the following modifications in the 5′-3′ direction:
2′-O-methyl modification; 2′-O-methyl modification and 2′-fluoro modification or;
2′-fluoro modification; 2′-O-methyl modification and 2′-O-methyl modification or;
2′-O-methyl modification; 2′-O-methyl modification and 2′-O-methyl modification; 2′-O-methyl modification.

12. (canceled)

13. (canceled)

14. The siNA agent for use according to claim 1, wherein the siNA is selected from double stranded RNA, small interfering RNA or short hairpin RNA.

15. The siNA agent for use according to claim 1, wherein the siNA is small interfering RNAs.

16. The siNA agent for use according to claim 1, wherein the siNA is asymmetric.

17. The siNA agent for use in the treatment of ophthalmic diseases in humans according to claim 1, wherein
the antisense ribonucleotide sequence is at least 90% identical to the sequences of the following list: SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95, SEQ ID No. 96, SEQ ID No. 97, SEQ ID No. 98, SEQ ID No. 99, SEQ ID No. 100, SEQ ID No. 101, SEQ ID No. 102, SEQ ID No. 103, SEQ ID No. 104, SEQ ID No. 105, SEQ ID No. 106, SEQ ID No. 107, SEQ ID No. 108, SEQ ID No. 109, SEQ ID No. 110; SEQ ID No. 143, SEQ ID No. 144, SEQ ID No. 145, SEQ ID No. 146, SEQ ID No. 147, SEQ ID No. 148, SEQ ID No. 149, SEQ ID No. 150, SEQ ID No. 151, SEQ ID No. 152, SEQ ID No. 153, SEQ ID No. 154, SEQ ID No. 155, SEQ ID No. 156, SEQ ID No. 157, SEQ ID No. 158, SEQ ID No. 159, SEQ ID No. 160, SEQ ID No. 161, SEQ ID No. 162, SEQ ID No. 163, SEQ ID No. 164, SEQ ID No. 165, SEQ ID No. 166;
and
the sense ribonucleotide sequence that is complementary to the antisense ribonucleotide sequence is at least 90% identical to the sequences of the following list: SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 119, SEQ ID No. 120, SEQ ID No. 121, SEQ ID No. 122, SEQ ID No. 123, SEQ ID No. 124, SEQ ID No. 125, SEQ ID No. 126, SEQ ID No. 127, SEQ ID No. 128, SEQ ID No. 129, SEQ ID No. 130, SEQ ID No. 131, SEQ ID No. 132, SEQ ID No. 133, SEQ ID No. 134, SEQ ID No. 135, SEQ ID No. 136, SEQ ID No. 137, SEQ ID No. 138, SEQ ID No. 139, SEQ ID No. 140, SEQ ID No. 141, SEQ ID No. 142.

18. (canceled)

19. (canceled)

20. A method for treating or preventing ophthalmic diseases in a subject comprising administering the double stranded short interfering nucleic acid (siNA) agent described in claim 1 to a subject.

21. (canceled)

22. A pharmaceutical composition comprising at least one siNA agent as described in claim 1, and a pharmaceutical acceptable carrier.

23. The pharmaceutical composition according to claim 22, wherein said composition is administered by topical eye application, subconjunctival injection, intravitreal injection, retrobulbar injection, intracameral injection, subtenon injection or deposition, intravenous injection, intravenous infusion.

24. A composition for inhibiting the expression of the dopamine-beta-hydroxylase gene in a cell wherein the composition comprises a siNA agent, wherein said siNA agent comprises an antisense ribonucleotide sequence arranged to base-pair to a substantially complementary sense ribonucleotide sequence;
wherein all of the nucleotides of the sense strand and all of the nucleotides of the antisense strand comprise a 2′-O-methyl modification or a 2′-fluoro modification;
wherein both the sense strand and the antisense strand independently comprise at least one phosphorothioate internucleotide linkage;
wherein the antisense ribonucleotide sequence is substantially complementary to a sense ribonucleotide sequence except in up to 6 nucleotides;
wherein the sense strand comprises 15 to 21 nucleotides;
wherein each nucleotide or each of the first 18 contiguous nucleotides in the 5′-3′ direction comprise a modification that is different from the modification of the adjacent nucleotide;
and wherein the first modification in the antisense strand is a 2′-O-methyl modification.

25. (canceled)

26. A method for treating or preventing ophthalmic diseases in a subject comprising administering the composition described in claim 1 to a subject.

27. A kit comprising the double stranded short interfering nucleic acid (siNA) agent according to claim 1, for inhibiting the expression of the dopamine-beta-hydroxylase gene in a cell according to the previous claim.

28. The double stranded siNA agent according to claim 1 wherein the antisense ribonucleotide sequence is identical to the sequences of the following list: SEQ ID No. 89, SEQ ID No. 90, SEQ ID No. 91, SEQ ID No. 93, SEQ ID No. 97, SEQ ID No. 98, SEQ ID No. 99, SEQ ID No. 101, SEQ ID No. 105, SEQ ID No. 106, SEQ ID No. 107, SEQ ID No. 109, SEQ ID No. 145, SEQ ID No. 146, SEQ ID No. 147, SEQ ID No. 149, SEQ ID No. 153, SEQ ID No. 154, SEQ ID No. 155, SEQ ID No. 157, SEQ ID No. 161, SEQ ID No. 162, SEQ ID No. 163, SEQ ID No. 165, SEQ ID No. 166; and
the sense ribonucleotide sequence is identical to the sequences of the following list: SEQ ID No. 81, SEQ ID No. 82, SEQ ID No. 83, SEQ ID No. 85, SEQ ID No. 121, SEQ ID No. 122, SEQ ID No. 123, SEQ ID No. 125, SEQ ID No. 129, SEQ ID No. 130, SEQ ID No. 131, SEQ ID No. 133, SEQ ID No. 137, SEQ ID No. 138, SEQ ID No. 139, SEQ ID No. 141, SEQ ID No. 142.

29. The method according to claim 26 for use in the treatment or prevention of ophthalmic diseases susceptible of being improved or prevented by inhibition of noradrenaline production.

30. The method according to claim 26 for use in the treatment or prevention of progressive optical neuropathy associated with the elevation of intraocular pressure.

31. The method according to claim 26 for use in the treatment or prevention of preferably diabetic retinopathy, infections, inflammation, uveitis or glaucoma.
